(19) United States
(12) Patent Application Publication
(10) Pub. No.: US 2013/0252831 A1
(43) Pub. Date: Sep. 26, 2013

(54) METHOD OF DIAGNOSING EARLY STAGE NON-SMALL CELL LUNG CANCER

(71) Applicant: H. Lee Moffitt Cancer Center and Research Institute, Inc., Tampa, FL (US)

(72) Inventor: Dung-Tsa Chen, Tampa, FL (US)

(73) Assignee: H. Lee Moffitt Cancer Center and Research Institute, Inc., Tampa, FL (US)

(21) Appl. No.: 13/891,433

(22) Filed: May 10, 2013

Related U.S. Application Data
(63) Continuation of application No. PCT/US2011/060155, filed on Nov. 10, 2011.
(60) Provisional application No. 61/412,174, filed on Nov. 10, 2010.

(51) Int. Cl. C12N 15/10

(52) U.S. Cl. CPC C12N 15/1089
USPC 506/8

(57) ABSTRACT

A “malignancy-risk” (MR) gene signature score was developed with abundant proliferative genes using principal component analysis. This MR gene signature was shown to be a predictive and prognostic factor of overall survival in early-stage NSCLC. The malignancy-risk signature showed a significant association with OS, with poor survival seen in patients having a higher MR score and better survival seen in patients having a low MR score. As a prognostic factor, the MR gene signature showed a positive correlation with TNM stage, histologic grade, and smoking status. Combination of the MR signature with each clinical parameter often showed the best survival in the low MR group with good clinical outcome. The MR gene profile, tested with a PCA scoring method, discriminated overall survival in lung cancer patients was a predictor independent of pathological staging and other clinical parameters.

### CROSS REFERENCE TO RELATED APPLICATIONS

This application claims priority to U.S. Provisional Application No. 61/412,174 entitled “Validation of Malignancy-Risk Gene Signature in Early-Stage Lung Cancer”, filed Nov. 10, 2010, the contents of which are hereby incorporated by reference into this disclosure.

### STATEMENT OF GOVERNMENT SUPPORT

This invention was made with government support under Grant No. CA119997; Grant No. CA076292; and Grant No. CA112215 awarded by the National Institutes of Health (NIH). The government has certain rights in the invention.

### FIELD OF THE INVENTION

This invention relates to cancer diagnosis. Specifically, the invention provides a novel method of diagnosing neoplastic diseases and dysfunctions using gene expression scoring.

### BACKGROUND OF THE INVENTION

Lung cancer is one of the most common causes of cancer-related death worldwide, accounting for more than one million deaths each year. Non-small cell lung cancer (NSCLC) accounts for 80-90% of all lung cancers (Wahbah, et al.; Changing trends in the distribution of the histologic types of lung cancer: a review of 4,439 cases. Ann Diagn Pathol. April 2007; 11(2):89-96). The primary treatment for early stage NSCLC is surgery. However, 30-50% patients experience relapse after resection and die of metastatic recurrence (Shedden, et al., Gene expression-based survival prediction in lung adenocarcinoma: a multi-site, blinded validation study. Nat Med. August 2008; 14(8):822-827). Five-year survival rates for early-stage I&II NSCLC range from 40% to 70% (Booth, et al, Adoption of adjuvant chemotherapy for non-small-cell lung cancer: a population-based outcomes study. J Clin Oncol. Jul. 20; 28(21):3472-3478).

Adjuvant chemotherapy (ACT) has become the standard treatment for patients with resected stage II-III NSCLC. (Pisters, K M, W K Evans, C G Azzoli, M G Kris, C A Smith, C E Desch, M R Somerfield, M C Brouwers, G Darling, P M Ellis, L E Gaspar, H I Pass, D R Spigel, J R Strawn, Y C Ung, and F A Shepherd, Cancer Care Ontario and American Society of Clinical Oncology adjuvant chemotherapy and adjuvant radiation therapy for stages I-IIIA resectable non small-cell lung cancer guideline. J Clin Oncol. 2007, 25(34): p. 5506-18) Several international clinical trials have demonstrated that adjuvant chemotherapy significantly improves the survival of patients with early-stage disease, such as 5% absolute benefit at 5 year survival in the Lung Adjuvant Cisplatin Evaluation (LACE) trial (Pignon J P, Tribodet H, Scagliotti G V, et al. Lung adjuvant cisplatin evaluation: a pooled analysis by the LACE Collaborative Group. J Clin Oncol. Jul. 20, 2008; 26(21):3552-3559), a 4% survival advantage at 5 years in the International Adjuvant Lung Trial (IALT) (Arriagada, et al., Cisplatin-based adjuvant chemotherapy in patients with completely resected non-small-cell lung cancer. N Engl J Med. Jan. 22, 2004; 350(4):351-360), a 15% survival advantage at 5 years in the JBR.10 trial (Waller, et al., Chemotherapy for patients with non-small cell lung cancer: the surgical setting of the Big Lung Trial. Eur J Cardiothorac Surg. July 2004; 26(1):173-182), a 9% survival advantage at 5 years in the Adjuvant Navelbine International Trialist Association (ANITA) trial (Douillard, et al., Adjuvant vinorelbine plus cisplatin versus observation in patients with completely resected stage IB-IIIA non-small-cell lung cancer (Adjuvant Navelbine International Trialist Association [ANITA]): a randomised controlled trial. Lancet Oncol. September 2006; 7(9):719-727), and a 12% survival advantage of 4 years in the carboplatin-based regimen trial (CALGB 9633) (Strauss, et al., Adjuvant paclitaxel plus carboplatin compared with observation in stage IB non-small-cell lung cancer: CALGB 9633 with the Cancer and Leukemia Group B, Radiation Therapy Oncology Group, and North Central Cancer Treatment Group Study Groups. J Clin Oncol. Nov. 1, 2008; 26(31):5043-5051). However, with a 4-15% survival advantage at 5 years from recent multinational clinical trials, not all patients benefit from ACT. (Pignon, J P, H Tribodet, G V Scagliotti, J Y Douillard, F A Shepherd, R J Stephens, A Dunant, V Torri, R Rosell, L Seymour, S G Spiro, E Rolland, R Fossati, D Aubert, K Ding, D Waller, and T Le Chevalier, Lung adjuvant cisplatin evaluation: a pooled analysis by the LACE Collaborative Group, J Clin Oncol. 2008, 26(21): p. 3552-9; Arrigada, R B Bergman, A Dunant, T Le Chevalier, J P Pingon, and J Vansteenkiste, Cisplatin-based adjuvant chemotherapy in patients with completely resected non-small-cell lung cancer. N Engl J Med, 2004, 350(4): p. 351-60; Waller, D, M D Peake, R J Stephens, N H Gower, R Milroy, M K Parmar, R M Rudd, and S G Spiro, Chemotherapy for patients with non-small cell lung cancer: the surgical setting of the Big Lung Trial. Eur J Cardiothorac Surg. 2004, 26(1): p. 173-82; Douillard, J Y, R Rosell, M De Lena, F Carpagnano, R Ramlau J L Gonzales-Larriba, T Grodzki, J R Pereira, A Le Groumellec, V Lorusso, C Clary, A J Torres, J Dahabreh, P J Souquet, J Astudillo, P Fournel, A Artal-Cortes, J Jassem, L Koubkova, P His, M Riggi, and P Hurteloup, Adjuvant vinorelbine plus cisplatin versus observation in patients with completely resected stage IB-IIIA non-small-cell lung cancer (Adjuvant Navelbine International Trialist Association [ANITA]): a randomised controlled trial. Lancet Oncol, 2006, 7(9): p. 719-27; Strauss, G M, J E Herndon, 2nd M A Maddaus, D W Johnstone, E A Johnson, D H Harpole, H H Gillenwater, D M Watson, D J Sugarbaker, R L Schilsky, E E Vokes, and M R Green, Adjuvant paclitaxel plus carboplatin compared with observation in stage IB non-small-cell lung cancer: CALGB 9633 with the Cancer and Leukemia Group B, Radiation Therapy Oncology Group, and North Central Cancer Treatment Group Study Groups, J Clin Oncol, 2008, 26(31): p. 5043-51). Given the morbidity associated with ACT, it is impervative to develop new prognostic tools to identify those patients with high probability of relapse. Such advances would improve patient selection in early stage NSCLC to optimize the potential benefits of ACT and minimize unnecessary treatment and associated morbidity.

Recent advances in molecular profiling have provided some insights into the importance of messenger RNA (mRNA) expression in cancer development (Wigle, et al., Molecular profiling of non-small cell lung cancer and correlation with disease-free survival. Cancer Res. Jun. 1, 2002; 62(11):3005-3008; Larsen, et al., Gene expression signature predicts recurrence in lung adenocarcinoma. Clin Cancer Res. May 15 2007; 13(10):2946-2954; Raponi, et al., Gene expression signatures for predicting prognosis of squamous cell and adenocarcinomas of the lung. Cancer Res. Aug. 1, 2006; 66(15):7466-7472; Kratz and Jablons, Genomic prognostic models in early-stage lung cancer. Clin Lung Cancer. May 2009; 10(3):151-157; Boutros, et al., Prognostic gene signatures for non-small-cell lung cancer. Proc. Natl Acad Sci USA. Feb. 24, 2009; 106(8):2824-2828, Roepman, et al, An immune response enriched 72-gene prognostic profile for early-stage non-small-cell lung cancer. Clin Cancer Res. Jan. 1, 2009; 15(1):284-290).

Numerous gene signatures have been developed to classify lung cancer patients with different clinical outcomes. (Boutros P C, Lau S K, Pintilie M. et al. Prognostic gene signatures for non-small-cell lung cancer. Proc Natl Acad Sci USA 2009; 106(8):2824-8; Roepman P, Jassem J, Smit E F, et al. An immune response enriched 72-gene prognostic profile for early-stage non-small-cell lung cancer. Clin Cancer Res 2009; 15(1):284-90; Chen H Y, Yu S L, Chen C H, et al. A five-gene signature and clinical outcome in non-small-cell lung cancer. N Engl J Med 2007; 356(1):11-20; Skrzypski M, Jassem E, Taron M, et al. Three-gene expression signature predicts survival in early-stage squamous cell carcinoma of the lung. Clin Cancer Res 2008; 14(15):4794-9; Sun Z, Wigle D A, Yang P. Non-overlapping and non-cell-type-specific gene expression signatures predict lung cancer survival. J. Clin Oncol 2008; 26(6):877-83; Baty F, Facompre M, Kaiser S, et al. Gene profiling of clinical routine biopsies and prediction of survival in non-small cell lung cancer. American journal of respiratory and critical care medicine 2010; 181(2):181-8; Wan Y W, Sabbagh E, Raese R, et al. Hybrid models identified a 12-gene signature for lung cancer prognosis and chemoresponse prediction. PLoS One 2010; 5(8):e12222; Kadara H, Lacroix L, Behrens C, et al. Identification of gene signatures and molecular markers for human lung cancer prognosis using an in vitro lung carcinogenesis system. Cancer Prev Res (Phila) 2009; 2(8):702-11; Xie Y, Xiao G, Coombes K, et al. Robust Gene Expression Signature from Formalin-Fixed Paraffin-Embedded Samples Predicts Prognosis of Non-Small-Cell Lung Cancer Patients. Clinical cancer research: an official journal of the American Association for Cancer Research 2011; Raz D J, Ray M R, Kim J Y, et al. A multigene assay is prognostic of survival in patients with early-stage lung adenocarcinoma. Clin Cancer Res 2008; 14(17):5565-70.)

There are some gene signatures derived from breast cancer that have prognostic value for lung cancer or are associated with lung metastasis. (Liu R, Wang X, Chen G Y, et al. The prognostic role of a gene signature from tumorigenic breast-cancer cells. N Engl J Med 2007; 356(3):217-26; Wan Y W, Qian Y, Rathnagiriswaran S, et al. A breast cancer prognostic signature predicts clinical outcomes in multiple tumor types. Oncology reports 2010; 24(2):489-94; Minn A J, Gupta G P, Siegel P M, et al. Genes that mediate breast cancer metastasis to lung. Nature 2005; 436(7050):518-24.)

Expression patterns of mRNA may provide molecular phenotyping that identify distinct classifications not evident by traditional histopathological methods and benefit early stage patterns for adjuvant chemotherapy assignment in lung cancer. Several studies have identified potential biomarkers and gene signatures for classifying lung cancer patients with significantly different clinical outcomes, such as KRAS mutations (Pao, et al. KRAS mutations and primary resistance of lung adenocarcinomas to gefitinib or erlotinib. PLoS Med. January 2005; 2(1):e17; Suda, et al., Biological and clinical significance of KRAS mutations in lung cancer an oncogenic driver that contrasts with EGFR mutation. Cancer Metastasis Rev. Mar; 29(1):49-60), ERCC1 (Tibaldi, et al., Correlation of CDA, ERCC1, and XPD polymorphisms with response and survival in gemcitabine/cisplatin-treated advanced non-small cell lung cancer patients. Clin Cancer Res. Mar. 15, 2008; 14(6):1797-1803), RRM1 (Rosell, et al., Ribonucleotide reductase messenger RNA expression and survival in gemcitabine/cisplatin-treated advanced non-small cell lung cancer patients. Clin Cancer Res. Feb. 15, 2004; 10(4):1318-1325), beta-tubulin 3 (Rosell, et al., Transcripts in pretreatment biopsies from a three-arm randomized trial in metastatic non-small-cell lung cancer. Oncogene, Jun. 5, 2003; 22(23):3548-3553), EGFR (Paez, et al., EGFR mutations in lung cancer; correlation with clinical response to gefitinib therapy. Science, Jun. 5, 2004; 304(5676):1497-1500; Gazdar et al., Mutations and addiction to EGFR; the Achilles ‘heal’ lung cancers? Trends Mol Med. October 2004; 10(10):481-486; Oshita, et al., Novel heterduplex method using small cytology specimens with a remarkably high success rate for analysing EGFR gene mutations with a significant correlations to gefitinib efficacy in non-small-cell lung cancer. Br J Cancer. Oct. 23, 2006; 95(8):1070-1075), and p27 (Filipits, et al., Cell cycle regulators and outcome of adjuvant cisplatin-based chemotherapy in completely resected non-small-cell lung cancer: the International Adjuvant Lung Cancer Trial Biologic Program. J Clin Oncol. Jul. 1, 2007; 25(19):2735-2740).

The inventors previously defined a malignancy-risk gene signature that is rich in genes involved in cell proliferation and is associated with cancer risk in normal breast tissue, as well as a prognostic factor for breast cancer. (Chen D T, Nasir A, Culhane A, et al. Proliferative genes dominate malignancy-risk gene signature in histologically-normal breast tissue. Breast cancer research and treatment 2010; 119(2):335-46.) Since the proliferative program of gene expression may be the earliest detectable event in normal tissues at risk for developing cancer, the “malignancy-risk” gene signature was evaluated to determine whether the signature is a prognostic factor of overall survival in early-stage NSCLC.

### SUMMARY OF THE INVENTION

A novel malignancy-risk gene signature comprised of numerous proliferative genes and having prognostic and predictive value for early-stage non-small cell lung cancer (NSCLC) patients is described.

The ability of the malignancy-risk gene signature to predict overall survival (OS) of early-stage NSCLC patients was tested using a large NSCLC microarray dataset from the Director's Challenge Consortium (n=442) and two independent NSCLC microarray datasets (n=117 and 133, for the GSB13213 and GSB14814 datasets, respectively). An overall malignancy-risk score was generated by principal component analysis to determine the prognostic and predictive value of the signature. An interaction model was used investigate a statistically significant interaction between adjuvant chemotherapy (ACT) and the gene signature. All statistical rests were two-sided.

The malignancy-risk gene signature was statistically significantly associated with OS (P<0.001) of NSCLC patients. Validation with the two independent datasets demonstrated that the malignancy-risk score had prognostic and predictive values: of patients who did not receive ACT, those with a low malignancy-risk score had increased OS compared with a high malignancy-risk score (P=0.007 and 0.01 for the GSE13212 and GSE14814 data sets, respectively), indicating a prognostic value; and in the GSE14814 dataset, patients receiving ACT survived longer in the high malignancy-risk score group (P=0.03) and a statistically significant interaction between ACT and the signature was observed (P=0.02).

The malignancy-risk gene signature was associated with OS and was a prognostic and predictive indicator. The malignancy-risk gene signature is useful to improve prediction of OS and to identify those NSCLC patients who will benefit from ACT.

### BRIEF DESCRIPTION OF THE DRAWINGS

For a fuller understanding of the invention, reference should be made to the following detailed description, takes in connection with the accompanying drawings, in which:

FIG. 1 is a table listing the malignancy-risk genes (94 genes with 102 probe sets) in the Affymetrix 133A chip, as listed in the NetAffx database

FIG. 2 is a table of the descriptive statistics of clinical predictors and association of the malignancy-risk gene signature with overall survival (OS) within subgroups in the Director's Challenge Consortium dataset (n-422)

FIG. 3 is a series of images depicting principal component (PC) analysis of the malignancy-risk gene signature. Principal component analysis was performed for the malignancy-risk gene signature in three lung datasets (the Director's Challenge Consortium [Director's], the GSE13213 dataset, and GSE14814 dataset) and in one breast dataset (GSE10780). A) Variation of the first five principal components in the four datasets and B) Pearson correlation of the loading coefficients from the first principal component in the four datasets are shown. r=Pearson correlation.

FIG. 4 is a graph depicting the association of the malignancy-risk gene signature with overall survival. A malignancy-risk score was generated for each patient from the Director's Challenge Consortium (n=442) by principal component analysis to reflect the combined expression of the malignancy-risk genes. High and low malignancy-risk groups were determined on the basis of a median split. Kaplan-Meier curves of overall survival are shown in the two groups with corresponding 95% confidence intervals (CIs) as error bars. A statistically significant difference of the Kaplan-Meier survival curves between the high and low malignancy-risk groups was determined by the two-sided log-rank test. The number of patients at risk is listed below the survival curves. MST=median survival time.

FIG. 5 is a series of images depicting the association of the malignancy-risk gene signature with histologic grade and TNM staging system. The malignancy-risk score was calculated for patients from the Director's Challenge Consortium for whom data on A) histological grade or B) TNM stage were available (n=435 and 439, respectively). Boxplot was used to display distribution of the malignancy-risk score within each group. The bottom and top of each box are the lower and upper quartiles, respectively. The black band near the middle of the box is the median. The extreme of the lower whisker represents the lower quartile minus 1.5 times the interquartile range and the extreme of the higher whisker is the upper quartile plus 1.5 times the interquartile range. Any data points beyond the extremes of the whiskers are indicated by empty circles as outliers. Spearman correlation (r) was calculate dot determine if an increasing trend existed between the continuous malignancy-risk score and increasing histological grade and TNM stage. All statistical tests were two-sided.

FIG. 6 is a series of images depicting the association of the malignancy-risk gene signature with other clinical predictors in the Director's Challenge Consortium dataset. Spearman's correlation (r) test was used to evaluate the association between the malignancy-risk score and A) smoking history (never, past, or current), B) pathologic T stage (T1, T2, or T3-T4), or C) pathologic N stage (N0, N1, or N2) to test any increasing trend. A two-sample Student t test was used to determine if differences between subgroups stratified by D) adjuvant radiotherapy (RT, no or yes), E) adjuvant chemotherapy (ACT, no or yes), or G) gender (female or male) were statistically significant. As shown in F) differences in grade (well-differentiated, moderately differentiated, or poorly differentiated) was not statistically significant. All statistical tests were two-sided.

FIG. 7 is a series of images depicting the analysis of the association between the malignancy-risk gene signature and overall survival by TNM stage. A) Kaplan-Meier curves of overall survival for patients from the Director's Challenge Consortium for whom data on TNM stage was available (n=439) was stratified by TNM stage (IA, IB, II, and III). A malignancy-risk score was generated for each patient by principal component analysis to reflect the combined expression of the malignancy-risk genes. High and low malignancy-risk groups were determined on the basis of a median split. A statistically significant difference in the Kaplan-Meier survival curves between the low and high malignancy-risk groups for patients with TNM stage IB and III disease (B-C, respectively) was determined by the two-sided log-rank test. 95% confidence intervals (CIs) are indicated by error bars. The number of patients at risk is listed below the curves. MST=median survival time.

FIG. 8 is a series of images depicting the evaluation of the relationship between clinical predictors and overall survival. Kaplan-Meier curves of overall survival for patients from the Director's Challenge Consortium was stratified by each clinical predictor: A) adjuvant chemotherapy (ACT), B) adjuvant radiotherapy (RT), C) smoking history, D) pathologic N stage, E) pathologic T stage, F) histologic grade, and G) gender. A statistically significant difference in the Kaplan-Meier survival curves was determined by the two-sided log-rank test. Error bars represent the 95% confidence intervals.

FIG. 9 is an image depicting the analysis of the association between the malignancy-risk gene signature and overall survival in stage IB patients with past smoking history. Data from the Director's Challenge Consortium (n=100) was analyzed. A statistically significant difference in the Kaplan-Meier survival curves between the low and high malignancy-risk groups for patients with TNM stage IB and past smoking history was determined by the two-sided log-rank test. 95% confidence intervals (CIs) are also presented (error bars). The number of patients at risk is listed below the curves. MST=median survival time.

FIG. 10 is a series of images depicting the comparison of expression of malignancy-risk (MR) genes vs. non-MR genes. Distribution of P values for the change of expression of A) malignancy-risk genes and B) non-malignancy-risk genes are shown. All P values were calculated by the Cox model for each individual gene using the continuous expression level data in the Director's Challenge Consortium dataset.

FIG. 11 is a table listing the results of an investigation of consistency of the malignancy-risk (MR) genes between lung cancer (Director's Challenge Consortium dataset) and breast cancer (GSE10780 dataset).

FIG. 12 is a series of images depicting the prognostic value of the malignancy-risk gene signature. A malignancy-risk score was generated using the loading coefficients of the first principal component from the Director's Challenge Consortium dataset for each patient. High and low malignancy-risk groups were determined on the basis of a median split using the medians of the malignancy-risk score from the Director's Challenge Consortium dataset. Kaplan-Meier curves of overall survival for patients who did not receive adjuvant chemotherapy or radiation therapy from A) the Director's Challenge Consortium (n=190), B) the GSE13213 dataset (n=117), and C) the GS14814 dataset from the JBR.10 trial (n=62) by high or low malignancy-risk group are shown. Error bars represent 95% confidence intervals (CIs). The two-sided log-rank test was done to calculate P. MST=median survival time.

FIG. 13 is a series of images depicting the predictive value of the malignancy-risk gene signature. A malignancy-risk score was generated using the loading coefficients of the first principal component from the Director's Challenge Consortium dataset for each patient. High and low malignancy-risk groups were determined on the basis of a median split using the median of the malignancy-risk score from the Director's Challenge Consortium dataset. Kaplan-Meier curves of overall survival for patients in the GSE14814 dataset from A) the high malignancy-risk group and B) the low malignancy-risk group by the adjuvant chemotherapy (ACT) or the observation cohort (OBS) are shown. The two-sided log-rank test was used to calculate P. Error bars represent 95% confidence intervals (CIs). MST=median survival time.

FIG. 14 is a series of images depicting the evaluation of the predictive value of the malignancy-risk gene signature in the Director's Challenge Consortium dataset. A) Kaplan-Meier curves of overall survival for patients from the Director's Challenge Consortium for whom data was available (n=322) was stratified by adjuvant chemotherapy (ACT) use (yes or no). Analyses of the association between overall survival (with 95% conscience intervals [CIs] represented as error bars) and adjuvant chemotherapy were also done for the B) the low malignancy-risk group and C) the high malignancy-risk group. High and low malignancy-risk groups were determined on the basis of a median split. A two-sided log-rank test was done to calculate P. MST=median survival time.

FIG. 15 is a graph depicting the association of the MR score with MR gene expressions.

FIG. 16 is a series of graphs depicting the prognostic effect of the MR signature at (a) MCLA cohort (p=0.004), (b) GSE14814 cohort (p=0.01), and (c) GSE13213 cohort (p=0.007).

FIG. 17 is a series of graphs depicting the predictive effect of the MS signature at GSE14814 cohort; (a) interaction effect (HR=0.29; p=0.02), (b) treatment effect in the high MR group (HR=0.48; p=0.03).

FIG. 18 is a series of images depicting the association of the MR signature with (a) grade (r=0.52; p<0.001), (b) stage (r=0.24; p<0.001), (c) smoking (p=0.27; p<0.001).

FIG. 19 is an image depicting that the MR signature could predict OS in Stage IB with past smoking patients.

FIG. 20 is a series of images depicting the association with drug sensitivity; (a) Cisplatin (r=−0.47; p=0.01), (b) Vinorelbine (r=−0.68; p<0.001), (c) association with treatment effect: lower MR in cisplatin treated group (p=0.08).

FIG. 21 is a table of the power analysis for the TCC cohort.

FIG. 22 is a table of statistical methods employed to analyze different types of drug sensitivity data in NCI-60 panel and MR scores.

FIG. 23 is a table listing a smaller subset of the malignancy-risk genes in the Affymetrix 133A chip, as listed in the NetAffx database

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

The term “about” as used herein is not intended to limit the scope of the invention but instead encompass the specified material, parameter or step as well as those that do not materially affect the basic and novel characteristics of the invention.

The inventors have demonstrated the malignancy-risk gene signature is a prognostic and predictive indicator in early-stage NSCLC. The original signature was derived from a comparison of normal breast tissue with invasive ductal carcinomas and is capable of discriminating molecularly-abnormal breast tissues that appear histologically normal from molecularly-normal breast tissues. (Chen D T, Nasir A, Culhane A, et al. Proliferative genes dominate malignancy-risk gene signature in histologically-normal breast tissue. Breast cancer research and treatment 2010; 119(2):335-46).

The original MR signature has showed clinical association with cancer relapse/progression, and prognosis in breast cancer. (Chen, D T, A Nasir, A Culhane, C Venkataramu, W Fulp, R Rubio, T Wang, D Agrawal, S M McCarthy, M Gruidl, G Bloom, T Anderson, J White, J Quackenbush, and T Yeatman, Proliferative genes dominate malignancy-risk gene signature in histologically-normal breast tissue. Breast Cancer Res Treat. 119(2): p. 335-46) A majority of the genes in the malignancy-risk signature are core regulators of the mammalian cell cycle and are essential for DNA replication and repair. (Bild A H, Yao G, Chang J T, et al. Oncogenic pathway signatures in human cancers as a guide to targeted therapies. Nature 2006; 439(7074):353-7).

This original MR signature develops using breast tissues was used to determine its applicability for determining prognosis and prediction of lung cancer. The application of the malignancy-risk gene signature to both breast and lung cancers is not surprising because sustained proliferative signaling has been considered one of the earliest and most fundamental hallmarks of cancer cells for the past decade. (Hanahan D, Weinberg R A. Hallmarks of cancer: the next generation. Cell 2011; 144(5):646-74). Expression of genes in the malignancy-risk gene signature may contribute to carcinogensis in lung and breast cancer. (Rosenwald A, Wright G, Wiestner A, et al. The proliferation gene expression signature is a quantitative integrator of oncogenic events that predicts survival in mantle cell lymphoma. Cancer Cell 2003; 3(2):185-97; Whitfield M L, George L K, Grant G D, et al. Common markers of proliferation. Nat Rev Cancer 2006; 6(2):99-106; Chung C H, Bernard P S, Perou C M. Molecular portraits and the family tree of cancer. Nat Genet 2002; 32 Suppl:533-40).

The signature includes 94 genes (102 probe sets on an Affymetrix 133 A chip). To make the signature as a useful clinical took the inventors used the first principal component by principal component analysis to derive an overall MR score to reflect the combined expression of the MR genes. The MR score is a linear weighted average expression among the MR genes where the weights are derived from the loading coefficients of the 1st principal component.

Several gene signatures have been developed to predict outcome NSCLC. (Boutros P C, Lau S K, Pintilie M, et al. Prognostic gene signatures for non-small-cell lung cancer. Proc Natl Acad Sci USA 2009; 106(8):2824-8; Roepman P, Jassem J, Smit E F, et al. An immune response enriched 72-gene prognostic profile for early-stage non-small-cell lung cancer. Clin Cancer Res 2009; 15(1):284-90; Chen H Y, Yu S L, Chen C H, et al. A five-gene signature and clinical outcome in non-small-cell lung cancer. N Engl J Med 2007; 356(1):11-20; Skrzypski M, Jassem E, Taron M, et al. Three-gene expression signature predicts survival in early-stage squamous cell carcinoma of the lung. Clin Cancer Res 2008; 14(15):4794-9; Sun Z, Wigle D A, Yang P. Non-overlapping and non-cell-type-specific gene expression signatures predict lung cancer survival. J Clin Oncol 2008; 26(6):877-83; Baty F, Facompre M, Kaiser S, et al. Gene profiling of clinical routine biopsies and prediction of survival in non-small cell lung cancer. American journal of respiratory and critical care medicine 2010; 181(2):181-8; Wan Y W, Sabbagh E, Raese R, et al. Hybrid models identified a 12-gene signature for lung cancer prognosis and chemoresponse prediction. PLoS One 2010; 5(8):e12222; Kadara H, Lacroix L, Behrens C, et al. Identification of gene signatures and molecular markers for human lung cancer prognosis using an in vitro lung carcinogenesis system. Cancer Prev Res (Phila) 2009; 2(8):702-11; Xie Y, Xiao G, Coombes K, et al. Robust Gene Expression Signature from Formalin-Fixed Paraffin-Embedded Samples Predicts Prognosis of Non-Small-Cell Lung Cancer Patients. Clinical cancer research: an official journal of the American Association for Cancer Research 2011; Raz D J, Ray M R, Kim J Y, et al. A multigene assay is prognostic of survival in patients with early-stage lung adenocarcinoma. Clin Cancer Res 2008; 14(17):5565-70).

Generally these gene signatures are not composed of genes involved in proliferation and few malignancy-risk genes overlapped with these signatures. In fact, a common biology underlying these previously defined gene signatures has not been described. Nonetheless, the inventors show here that the malignancy-risk gene signature, a proliferative gene signature, is associated with both cancer risk and progression.

One might predict that a gene signature derived from the Director's Challenge Consortium dataset of lung cancers could have better prognostic predictive value than the malignancy-risk gene signature because there may be substantial differences between lung and breast cancer and the gene signature derived from the breast tissue may not be optimal for lung cancer. Surprisingly, a gene signature derived on the basis of high correlation with OS in the Director's Challenge Consortium dataset was prognostic but not predictive (data not shown).

Furthermore, a majority of genes in the malignancy-risk signature were absent in this signature, as has been reported for other gene signatures derived from this database. (Wan Y W, Sabbagh E, Raese R, et al. Hybrid models identified a 12-gene signature for lung cancer prognosis and chemoresponse prediction. PLoS One 2010; 5(8):e12222; Guo N L, Wan Y W, Bose S, et al. A novel network model identified a 13-gene lung cancer prognostic signature. International journal of computational biology and drug design 2011; 4(1):19-39). Why these strongly prognostic and predictive genes do not appear in these analyses is unclear. What is clear is that different approaches may lead to different gene signatures.

There are a few gene signatures developed in breast cancer and tested in lung cancer although they do not completely overlap with the malignancy-risk gene signature and are either a metastasis signature (Minn A J, Gupta G P, Siegel P M, et al. Genes that mediate breast cancer metastasis to lung. Nature 2005; 436(7050):518-24) or a prognostic signature (Liu R, Wang X, Chen G Y, et al. The prognostic role of a gene signature from tumorigenic breast cancer cells. N Engl J Med 2007; 356(3):217-26; Wan Y W, Qian Y, Rathnagiriswaran S, et al. A breast cancer prognostic signature predicts clinical outcomes in multiple tumor types. Oncology reports 2010; 24(2):489-94).

In contrast, the malignancy-risk gene signature features both prognostic and predictive factors in NSCLC and shares some unique clinical features in both lung and breast cancer. The expression of the majority of malignancy-risk genes was increased in breast cancer and also was associated with poorer survival in lung cancer. In addition, a strong correlation of the loading coefficients was reported between the two tumor types. The inventor's malignancy-risk gene signature described herein is the first to show such a high consistency of the gene signature in both tumor types. The original malignancy-risk gene signature showed clinical association with cancer relapse/progression, and prognosis in the breast cancer. (Chen D T, Nasir A, Venkataramu C, et al. Evaluation of malignancy-risk gene signature in breast cancer patients. Breast Cancer Res Treat 2010; 120(1):25-34). Similarly, the gene signature herein described demonstrated a statistically significant association with OS and other clinical predictors in NSCLC (TNM stage and histologic grade). Collectively, these findings suggest transferability of the malignancy-risk gene signature between breast and lung cancer, one unique feature not seen in other gene signatures derived for various tumor types. (Liu R, Wang X, Chen G Y, et al. The prognostic role of a gene signature from tumorigenic breast-cancer cells. N Engl J Med 2007; 356(3):217-26; Wan Y W, Qian Y, Rathnagiriswaran S, et al. A breast cancer prognostic signature predicts clinical outcomes in multiple tumor types. Oncology reports 2010; 24(2):489-94; Minn A J, Gupta G P, Siegel P M, et al. Genes that mediate breast cancer metastasis to lung. Nature 2005; 436(7050):518-24).

From a predictive aspect, the malignancy-risk gene signature has demonstrated the potential to identify early-stage NSCLC patients likely to benefit from ACT. A 15-gene signature described by Zhu et al. (Zhu C Q, Ding K, Strumpf D, et al. Prognostic and predictive gene signature for adjuvant chemotherapy in resected non-small-cell lung cancer. Journal of clinical oncology: official journal of the American Society of Clinical Oncology 2010; 28(29):4417-24) was the first predictive signature for ACT in resected NSCLC, derived from the randomized phase III JBR.10 trial (Winton T, Livingston R, Johnson D, et al. Vinorelbine plus cisplatin vs. observation in resected non-small-cell lung cancer. N Engl J Med 2005; 352(25):25898-97). However, that malignancy-risk gene signature also showed a statistically significant predictive value comparable with that on the RT-PCR basis reported by Zhu et al. with no overlap between the genes in both signatures. This observation suggests that the relationship between a survival benefit and ACT could be also affected expression of the genes included in the malignancy-risk gene signature. Specifically, the survival benefit from ACT relative to the observation cohort was considerably greater in the high malignancy-risk group.

In contrast, the survival benefit of ACT vs. the observation cohort was not statistically significant in the low malignancy-risk group; however, the observation cohort seemed to have the advantage in OS for the first two years compared with those receiving ACT. In addition, evaluation of the predictive value in the Director's Challenge Consortium dataset indirectly supported the utility of the signature although it was a retrospective study. Together, these results suggest that the malignancy-risk gene signature is a strong predictive factor for a differential OS benefit from ACT. Although recent multinational clinical trials (Pignon J P, Tribodet H, Scagliotti G V, et al. Lung adjuvant cisplatin evaluation; a pooled analysis by the LACE Collaborative Group, J Clin Oncol 2008; 26(21):3552-9; Arriagada R, Bergman B, Dunant A, et al. Cisplatin-based adjuvant chemotherapy in patients with completely resected non-small-cell lung cancer. N Engl J Med 2004; 350(4):351-60; Douillard J Y, Rosell R, De Lena M, et al. Adjuvant vinorelbine plus cisplatin versus observation in patients with completely resected stage IB-IIIA non-small-cell lung cancer (Adjuvant Navelbine International Trialist Association [ANITA]); a randomised controlled trial, Lancet Oncol 2006; 7(9):719-27; Strauss G M, Herndon J E, 2nd, Maddaus M A, et al. Adjuvant paclitaxel plus carboplatin compared with observation in stage IB non-small-cell lung cancer; CALGB 9633 with the Cancer and Leukemia Group B, Radiation Therapy Oncology Group, and North Central Cancer Treatment Group Study Groups. J Clin Oncol 2008; 26(31):5043-51; Winton T, Livingston R, Johnson D, et al. Vinorelbine plus cisplatin vs. observation in resected non-small-cell lung cancer, N Engl J Med 2005; 352(25):2589-97; Pisters K M, Evans W K, Azzoli C G, et al. Cancer Care Ontario and American Society of Clinical Oncology adjuvant chemotherapy and adjuvant radiation therapy for stages I-IIIa resectable non small-cell lung cancer guideline. J Clin Oncol 2007; 25(34):5506-18) have established that ACT is associated with improvement of OS in patients with early-stage NSCLC, the malignancy-risk gene signature may provide an additional tool to help identify a subset patients at high-risk of death who may benefit from ACT.

Similar to other prognostic signatures, the malignancy-risk gene signature was able to predict OS in NSCLC patients. (Roepman P, Jassem J, Smit E F, et al. An immune response enriched 72-gene prognostic profile for early-stage non-small-cell lung cancer. Clin Cancer Res 2009; 15(1):284-90; Chen H Y, Yu S L, Chen C H, et al. A five-gene signature and clinical outcome in non-small-cell lung cancer. N Engl J Med 2007; 356(1):11-20; Skrzypski M, Jassem E, Taron M, et al. Three-gene expression signature predicts survival in early-stage squamous cell carcinoma of the lung. Clin Cancer Res 2008; 14(15):4794-9; Sun Z, Wigle D A, Yang P. Non-overlapping and non-cell-type-specific gene expression signatures predict lung cancer survival. J Clin Oncol 2008; 26(6):877-83; Baty F, Facompre M, Kaiser S, et al. Gene profiling of clinical routine biopsies and prediction of survival in non-small cell lung cancer. American journal of respiratory and critical care medicine 2010; 181(2):181-8; Wan Y W, Sabbagh E, Raese R, et al. Hybrid models identified a 12-gene signature for lung cancer prognosis and chemoresponse prediction. PLoS One 2010; 5(8):e12222; Kadara H, Lacroix L, Behrens C, et al. Identification of gene signatures and molecular markers for human lung cancer prognosis using an in vitro lung carcinogensis system. Cancer Prev Res (Phila) 2009; 2(8):702-11; Raz D J, Ray M R, Kim J Y, et al. A multigene assay is prognostic of survival in patients with early-stage lung adenocarcinoma. Clin Cancer Res 2008; 14(17):5565-70; Liu R, Wang X, Chen G Y, et al. The prognostic role of a gene signature from tumorigenic breast-cancer cells. N Engl J Med 2007; 356(30):217-26).

Patients with a high malignancy-risk score tended to have shorter OS compared with those who had a low malignancy-risk score. In addition, subgroup analysis showed the malignancy-risk signature's value beyond the conventional clinical predictors with a statistically significant association of the gene signature with OS in one or more risk groups for each clinical predictor. In particular, the malignancy-risk gene signature was able to consistently distinguish between the two risk groups (low and high malignancy-risk groups, respectively, corresponding to good and poor OS) in the subgroups of stage IB patients, and stage IB patients who had a history of smoking. Because the benefit of ACT remains unclear in stage IB NSCLC, the signature has potential clinical application for stage IB patients, such as recommendation of ACT only for stage IB patients with a high malignancy-risk score.

Materials and Methods

Malignancy-Risk Gene Signature

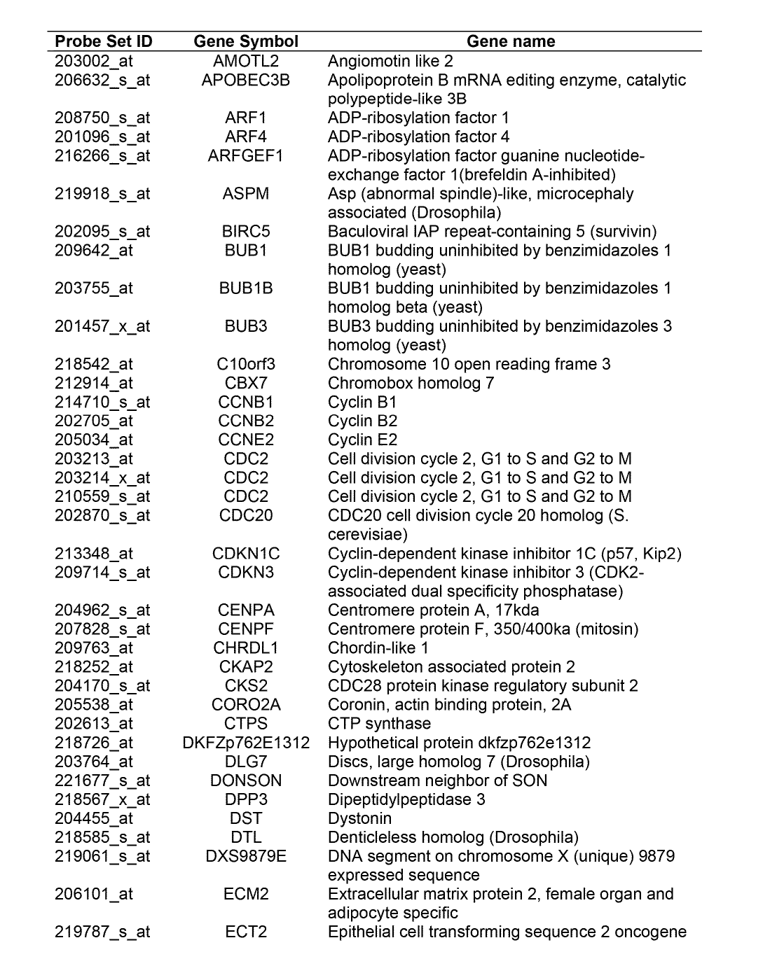

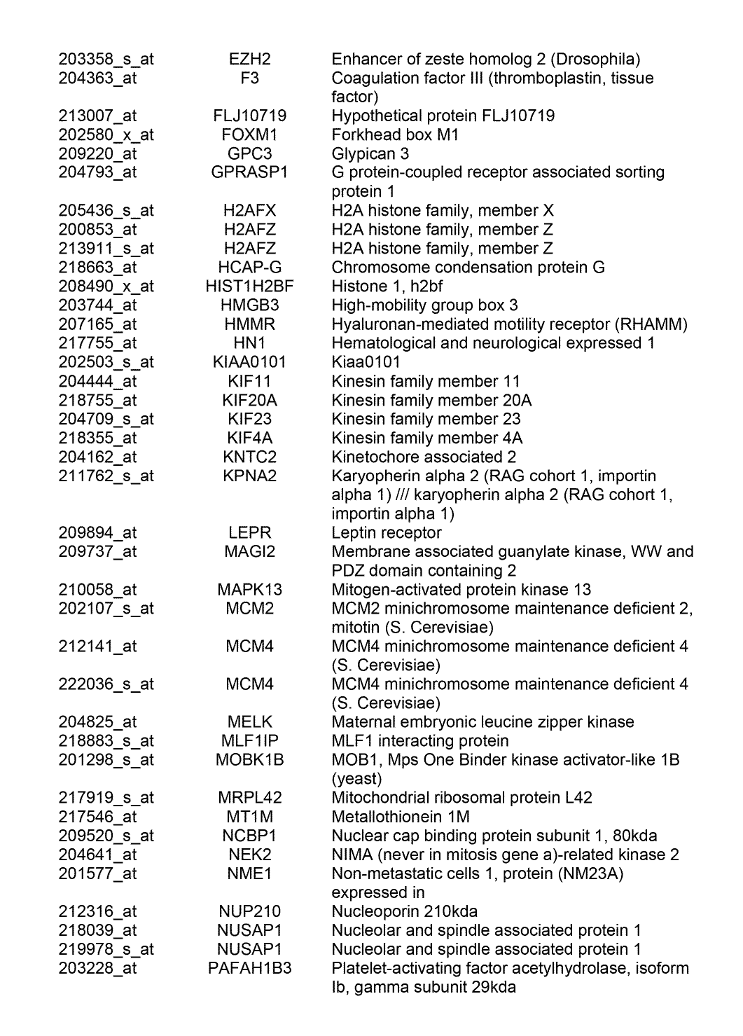

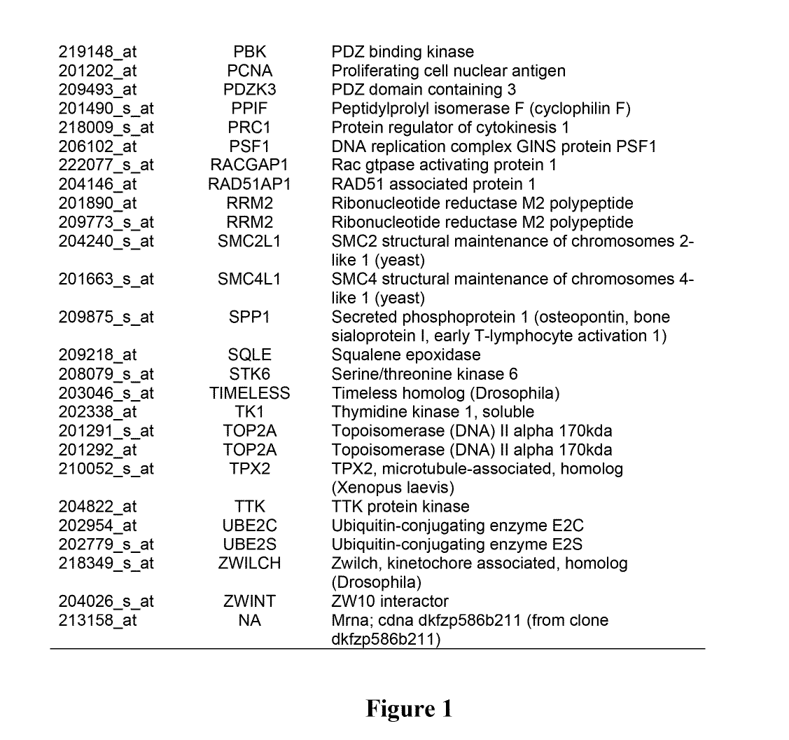

The “malignancy-risk” gene signature was derived from a comparison of normal breast tissues with breast cancer tissues and is capable of discerning molecularly-abnormal breast tissues that appear histologically normal. The signature includes 120 genes (140 probe sets on Affymetrix 133 Plus 2 chip), but its complexity is reduced to 94 genes (102 probe sets in Affymetrix 133A chip) (FIG. 1) This signature is predominantly composed of genes involved in proliferation (56 of the 94 malignancy-risk genes, 59.6%), consistent with the near universal loss of cell cycle control in the earliest stages of tumor development.

Microarray Datasets

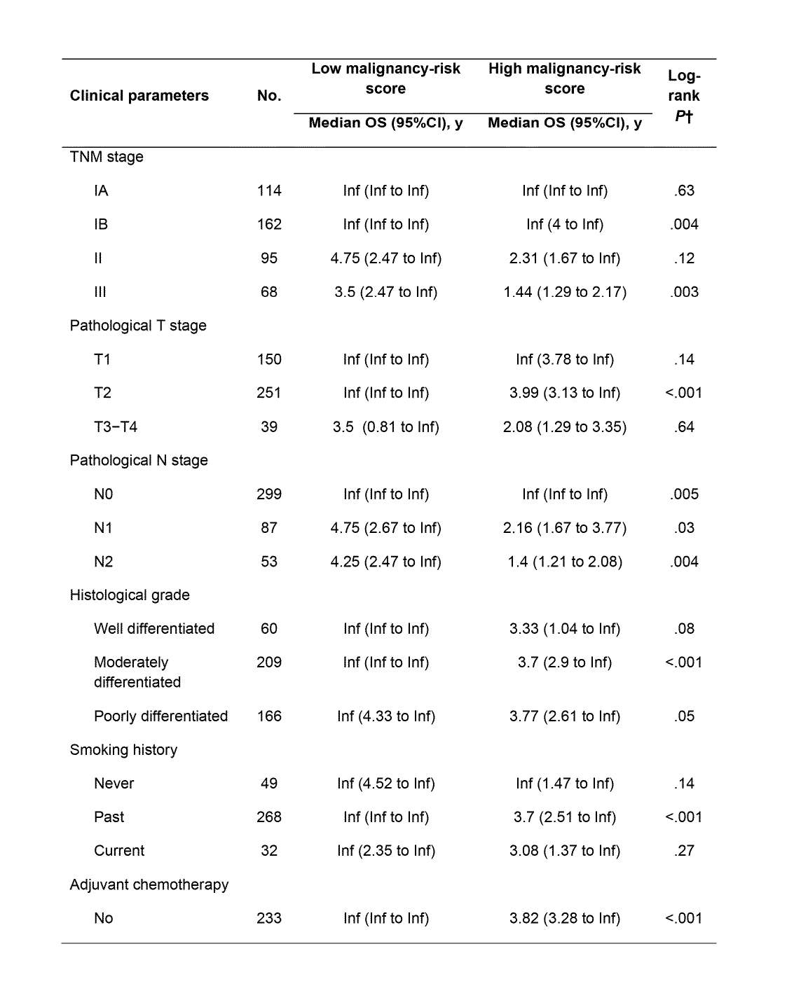

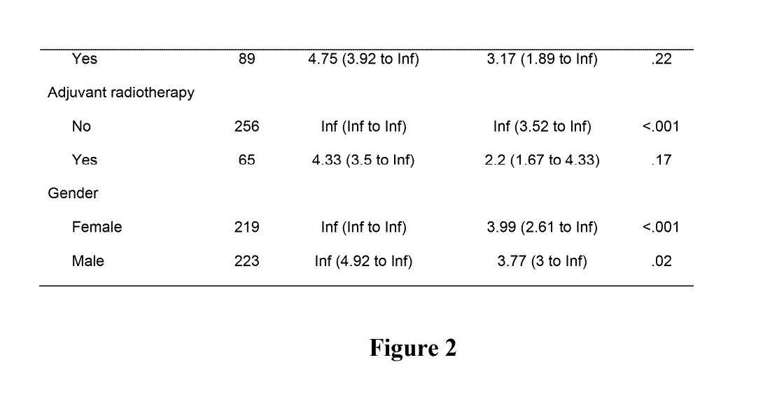

Data for the primary analysis were from the Director's Challenge Consortium for the Molecular Classification of Lung Adenocarcinoma (Shedden, et al., Gene expression-based survival prediction in lung adenocarcinoma: a multi-site, blinded validation study. Nat Med. August 2008; 14(8):822-827). This is, a large retrospective, multi-site microarray study for lung adenocarcinmoas. A total of 442 samples, were used for statistical analysis. Overall survival (censored at 5 years) was the primary outcome unable with a median follow-up of 3.92 years (255 samples were from patients who were alive and 187 samples were from patients who had died). Clinical predictors included TNM stage, T stage, N stage, pathologic grade, smoking history, ACT, adjuvant radiotherapy, and gender. (FIG. 2)

Two independent NSCLC microarray datasets and one breast cancer dataset were included to validate the malignancy-risk gene signature; GSE13213 (Tomida S, Takeuchi T, Shimada Y, et al. Relapse-related molecular signature in lung adenocarcinomas identifies patients with dismal prognosis. J Clin Oncol 2009; 27(17):2793-9); GSE14814 (Zhu C Q, Ding K, Strumpf D, et al. Prognostic and predictive gene signature for adjuvant chemotherapy in resected non-small-cell lung cancer. Journal of clinical oncology: official journal of the American Society of Clinical Oncology 2010; 28(29):4417-24); and GSE10780 (Chen D T, Nasir A, Culhane A, et al. Proliferative genes dominate malignancy-risk gene signature in histologically-normal breast tissue. Breast cancer research and treatment 2010; 119(2):335-46).

The GSE13213 dataset had 117 lung adenocarcinomas samples with overall survival (OS) information available (68 samples were from patients who were alive and 49 samples were from patients who had died). These 117 patients did not receive ACT and allows us to evaluate the prognostic value of the malignancy-risk gene signature. Because the dataset was generated from an Agilent cDNA array, we used gene symbols to identify the malignancy-risk genes for this dataset (116 probe sets for 87 genes).

The GSE14814 dataset (Affymetrix 133 A chip) was extracted from the JBR.10, a randomized controlled trial with two cohorts: patients who received ACT (n=71) vs. observation alone (n=62). Because the study was a randomized trial and data were collected in a prospective way, this dataset provides a unique opportunity to evaluate both prognostic and predictive features for the malignancy-risk gene signature.

For the GSE10780 dataset composed of 143 normal breast and 42 tumor samples, the inventors evaluated if genes patterns were consistent between breast and lung cancer (e.g., increase in the expression of genes in both cancer types).

Statistical Analysis

Data Normalization

Gene expression values were calculated using the robust multi-array average (RMA) algorithm (Irizarry, et a., Summaries of Affymetrix GeneChip probe level data. Nucleic Acids Res. Vol 31, 2003:e15) for the Director's Challenge Consortium, GSE14814 and GSE10780 datasets (Affymetrix gene chips) whereas the GSE13213 dataset was normalized by the Loess method (Yang Y H, Dudoit S, Luu P, et al. Normalization for cDNA microarray data: a robust composite method addressing single and multiple slide systematic variation. Nucleic acids research 2002; 30(4):e15).

Derivation of Malignancy-Risk (MR) Score

An overall malignancy-risk score was generated using principal component analysis to reflect the combined effect of the MR genes. Specifically, the first principal component (a weighted average expression among the malignancy-risk genes) was used, as it accounts for the largest variability in the data, to represent the overall expression level for the MR gene signature. That is, MR score=Σwᵢxᵢ, a weighted average expression among the MR genes, where xi represents gene i expression level, wi is the corresponding weight (loading coefficient) with Σwᵢ²=1, and the wi values maximize the variance of Σwᵢxᵢ. This approach has been used to derive the malignancy-risk gene signature in the inventors previously reported breast cancer study. (Chen, et al., Proliferative genes dominate malignancy-risk gene signature is histologically-normal breast tissue. Breast Cancer Res Treat. Jan; 119(2):335-346).

The median score for the 422 patients was found to be about 0.26. Malignancy-risk scores that are above about 0.26 are considered to be high scores while those below about 0.26 are considered to be low scores.

Association With OS and Other Clinical Parameters

The influence of the malignancy-risk gene signature was tested to see if the overall survival of two malignancy-risk groups (high and low) formed by a median-split of the malignancy-risk score were statistically significantly different. The two-sided log-rank test was used to calculate P values. Evaluation of the median-split malignancy-risk score as an independent factor predicting lung cancer prognosis was done by including several clinical predictors in multivariable Cox proportional hazards model: TNM stage (IA, IB, II, and III), grade (well, moderately, or poorly differentiated), ACT (yes or no), adjuvant radiotherapy (yes or no), gender (female and male), and smoking history (yes or no). The proportional hazards assumption was checked by the scaled Schoenfeld residual. (Granbsch P, Therneau T. Proportional hazards tests and diagnostics based on weighted residuals. Biometrika 1994; 81:515-26).

Multivariable analysis was also used to evaluate interactions between the high and low malignancy-risk groups and a clinical predictor after adjusting other clinical predictors. Spearman's correlation (r) analysis was used to test an increasing trend of the continuous malignancy-risk score with stage, grade, and smoking history. A two-sided log-rank test was used to determine if the malignancy-risk gene signature could predict OS within different malignancy-risk groups by clinical predictors (e.g., TNM stage IA, IB, and II-III) or risk groups jointly defined by all clinical predictors (e.g., TNM stage with smoking history).

Univariate Analysis

Cox proportional hazards model was used to examine association of each MR gene with OS. The scaled Schoenfeld residual was used to cheek the proportional hazards assumption. (Grambsch P, Therneau T. Proportional hazards tests and diagnostics based on weighted residuals. Biometrika 1994; 81:515-26). Fishers exact test was used to determine the overall statistical significance of the malignancy-risk genes (102 probe sets) by comparison with non-malignancy-risk genes (22181 probe sets). The two-sided P value was calculated by univariate analysis and was adjusted by the false discovery rate for multiple testing. (Benjamini Y, Hochberg Y. Controlling the false discovery rate: a practical and powerful approach to multiple testing. Journal of the Royal Statistical Society Series B-Methodological 1995; 57(1):289-300).

Evaluation of Prognostic and Predictive Features

According to the guideline by Clark (Clark G M. Prognostic factors versus predictive factors. Examples from a clinical trial of erlotinib. Molecular oncology 2008; 1(4):406-12), the inventors tested the prognostic value of the malignancy-risk gene signature on the patients without ACT for each of the three lung cancer datasets to see if those with either high or low malignancy-risk scores (high or low malignancy-risk group) had statistically significantly different OS as measured by the two-sided log-rank test. For the predictive value, treatment effect (compared with an observation cohort who did not receive ACT) was evaluated to determine any association with OS within each malignancy-risk group in the GSE14814 dataset. In addition, an interaction model was used to investigate a statistically significant interaction between ACT and the malignancy-risk gene signature which could suggest differential treatment effects among those in the high or low malignancy-risk groups.

Because the microarray platforms were different among the three NSCLC datasets, gene level data were used for evaluation (a gene expression level was defined as an average of the expression level for a set of probe sets for the same gene; any probe set with a missing value was excluded). As a result, 87 malignancy-risk genes were identified in all datasets to evaluate the predictive value of the signature. Eighty-two patients were excluded in the Director's Challenge Consortium data for the evaluation of the prognostic and predictive values since they were included in the GSE14814 dataset. Before analysis, data were standardized by centering the mean and scaled by the standard deviation for each gene in each dataset. Principal component analysis was first implemented on the Director's Challenge Consortium data to obtain the malignancy-risk score was constructed on the basis of the loading coefficients from the first principal component. The same loading coefficients were also used to compute the malignancy-risk score for the GSE13213 and GSE14814 datasets. The median of the malignancy-risk score in the Director's Challenge Consortium dataset was used as the cutoff to designate low and high malignancy-risk groups in each of the three datasets to test the prognostic and predictive values.

Results

Data from the Director's Challenge Consortium was used in the primary analysis of 1) the association between the malignancy-risk gene signature and OS, grade, TNM stage, and other clinical predictors; and 2) the gene signature effect within different risk groups by clinical predictors and the interaction between the two. A univariate analysis was also done. The other two long datasets were used to test the prognostic and predictive value of the malignancy risk signature.

Principal Component Analysis

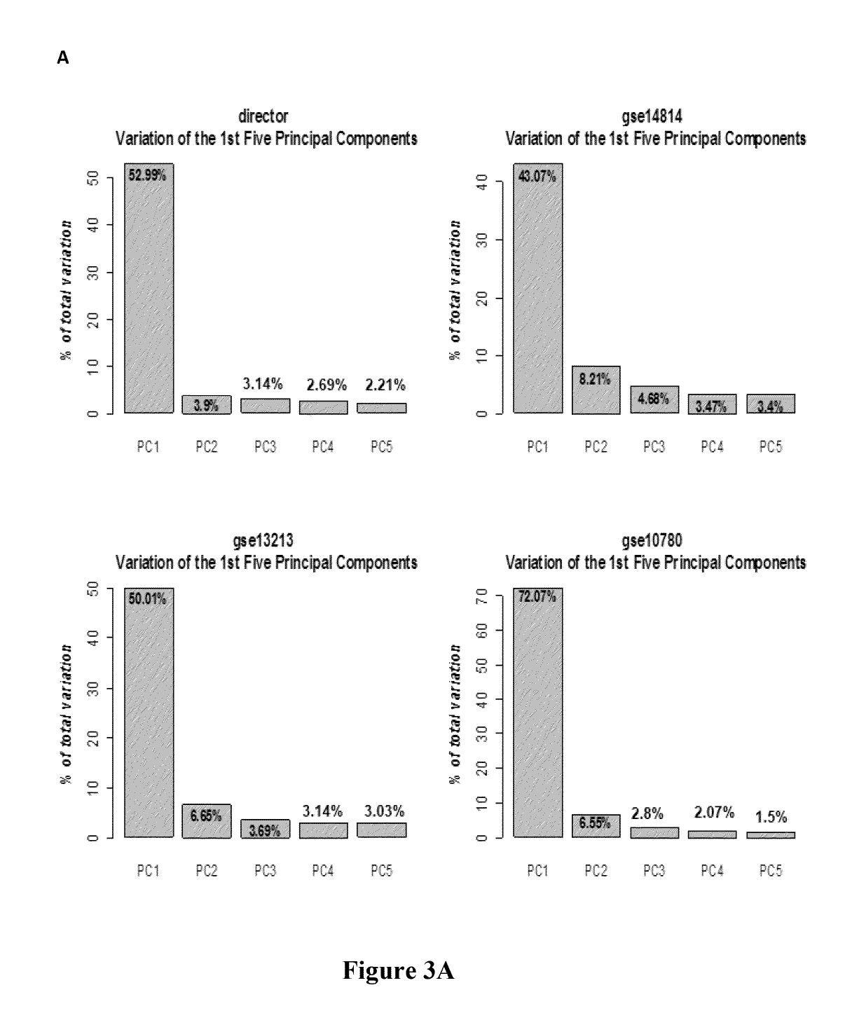

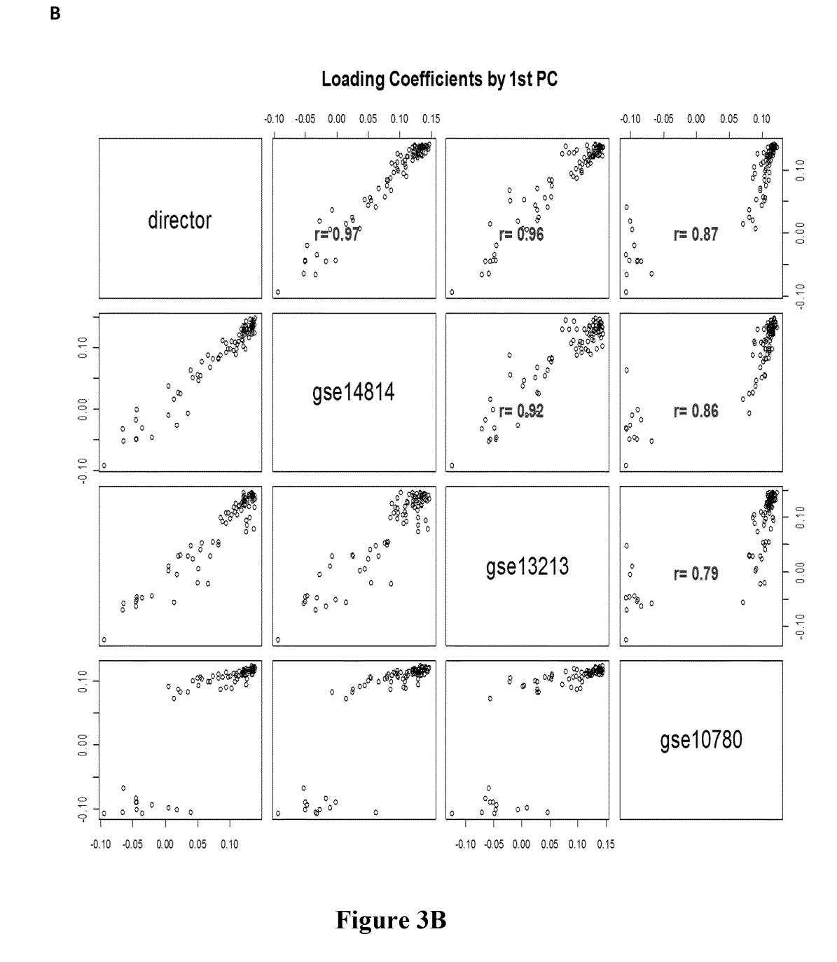

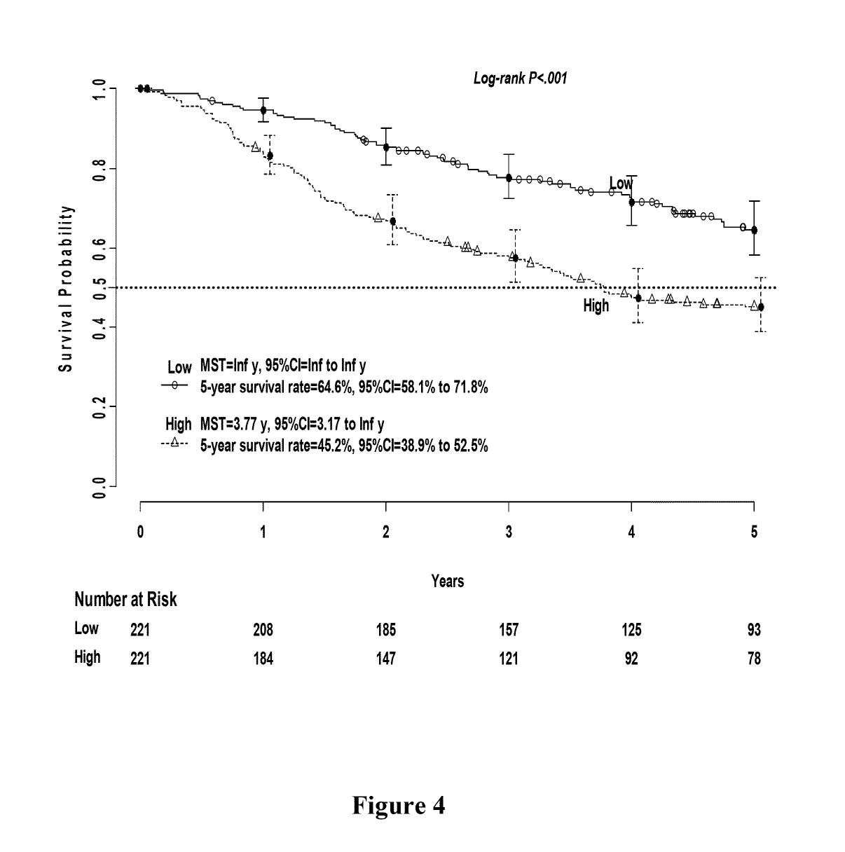

The malignancy-risk gene signature was analyzed using principal component analysis to evaluate the percent of variability and loading coefficients by the first principal component (i.e., the malignancy-risk score) for each of the four datasets. Results showed 43.1%-53% variability explained by the first principal component in three lung datasets and 72.1% variability in the breast cancer dataset (FIG. 3), suggesting the first principal component well represents the malignancy-risk gene signature. Pearson correlation of the loading coefficients was 0.92-0.97 among the three early-stage NSCLC datasets and 0.79-0.87 between the breast cancer dataset and due three early-stage NSCLC datasets, indicating transferability of the signature between breast cancer and lung cancer (FIG. 4).

Relationship Between the Malignancy-Risk Gene Signature and OS and Other Clinical Predictors

Division of lung cancer patients from Director's Challenge Consortium data-set into high vs. low malignancy-risk groups showed that patients in the high malignancy-risk group had statistically significantly shorter OS compared with those in the low malignancy-risk group (log-rank P<0.001; and hazard ratio [HR] of death=2.02, 95% confidence interval [CI]=1.5 to 2.72) (FIG. 4). The 5-year survival rate estimate for the high malignancy-risk group (5-year survival rate=45.2%, 95%; CI=38:9% to 52.5%) was less than that for the low malignancy-risk group (5-year survival rate=64.6%, 95%; CI=58.1% to 71.8%) and their 95% confidence intervals did not overlap (FIG. 4).

In multivariable analysis, the median-split malignancy-risk score was a statistically significant prognostic predictor (P<0.001) after adjusting for clinical predictors, including TNM stage, grade, smoking history, gender, and adjuvant treatments (HR=2.14, 95% CI=1.42 to 3.22 for high vs. low malignancy-risk groups). The assumption of proportional hazards was not rejected.

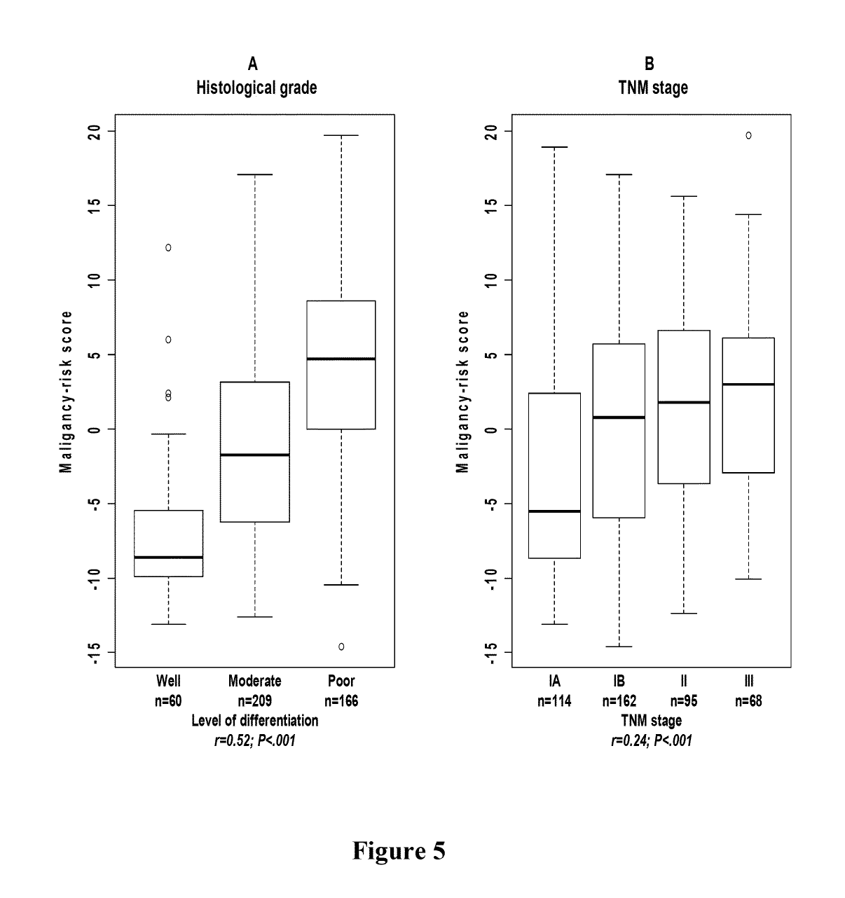

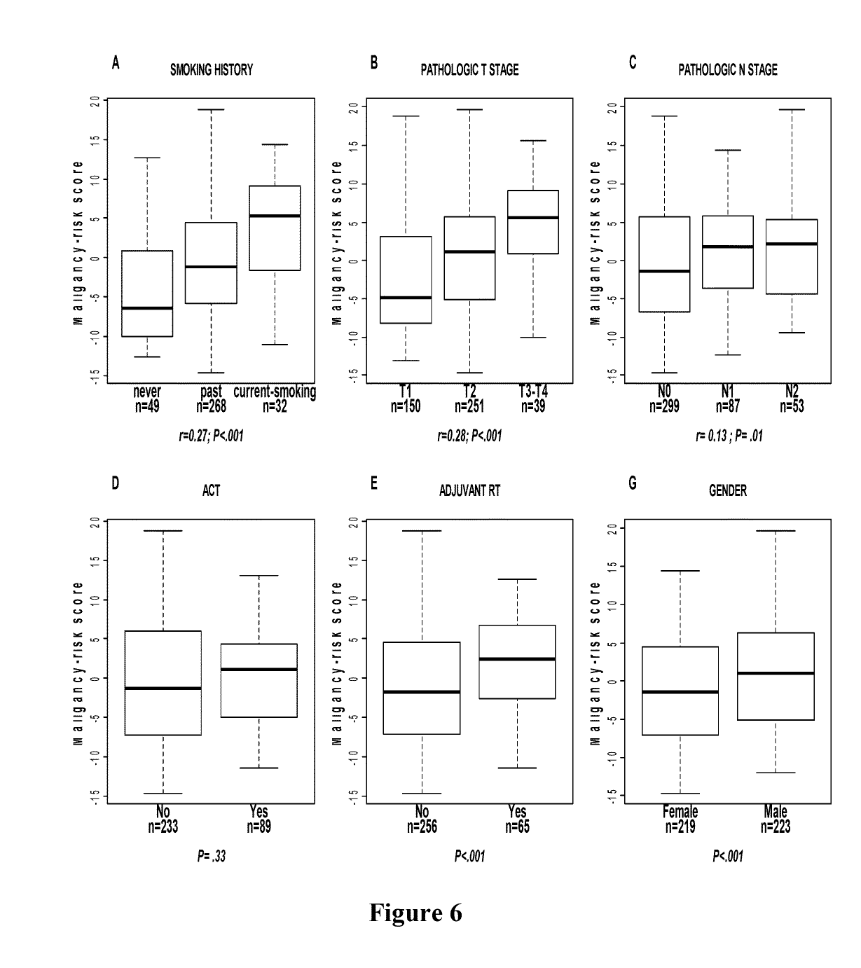

In relation to histological grade, an increasing trend from well to poorly differentiated tumors was observed for the malignancy-risk score (r=0.52, P<0.001) (FIG. 5A). A similar association between the malignancy-risk score and TNM stage (r=0.24, P<0.001) (FIG. 5B), pathological T stage (r=0.28, P<0.001), pathological N stage (r=0.13, P=0.01), and smoking history (r=0.27, P<0.001) was observed (FIG. 6).

Evaluation of the Signature Within Different Risk Groups by Clinical Predictors and a Measurement of the Potential Interaction

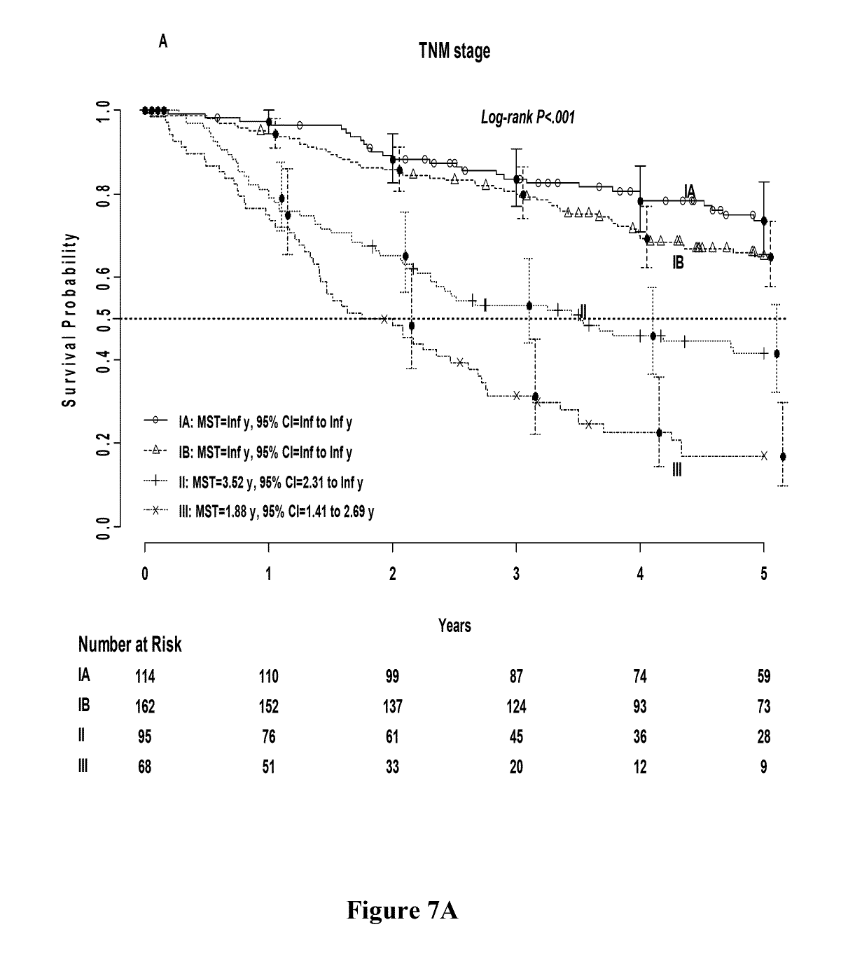

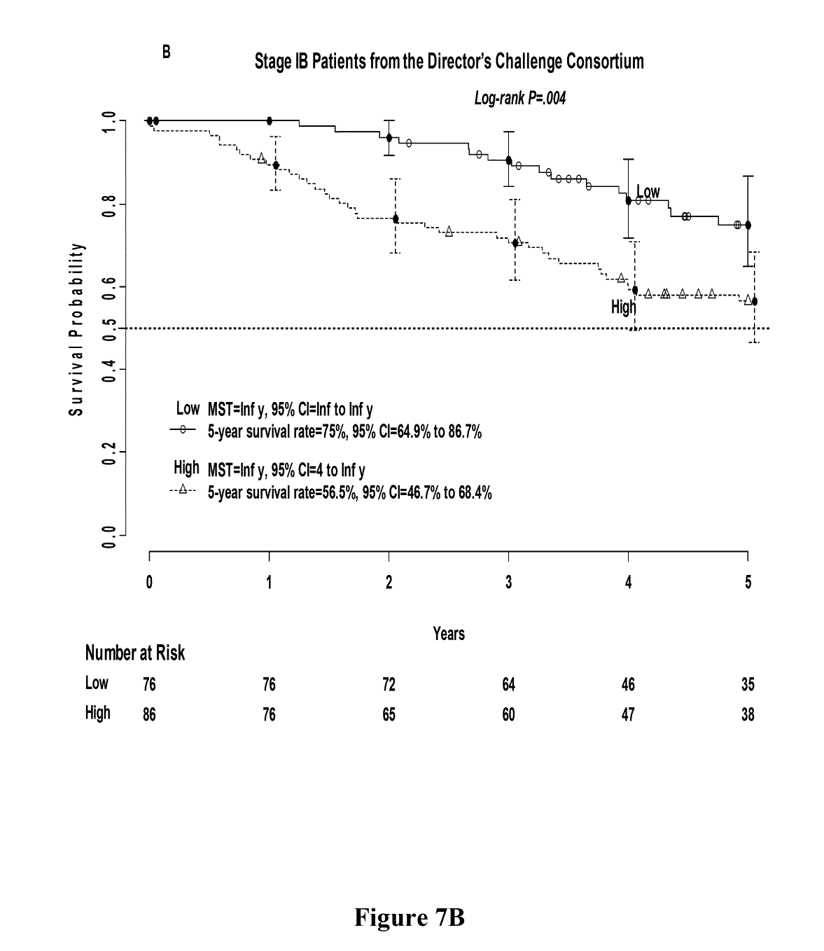

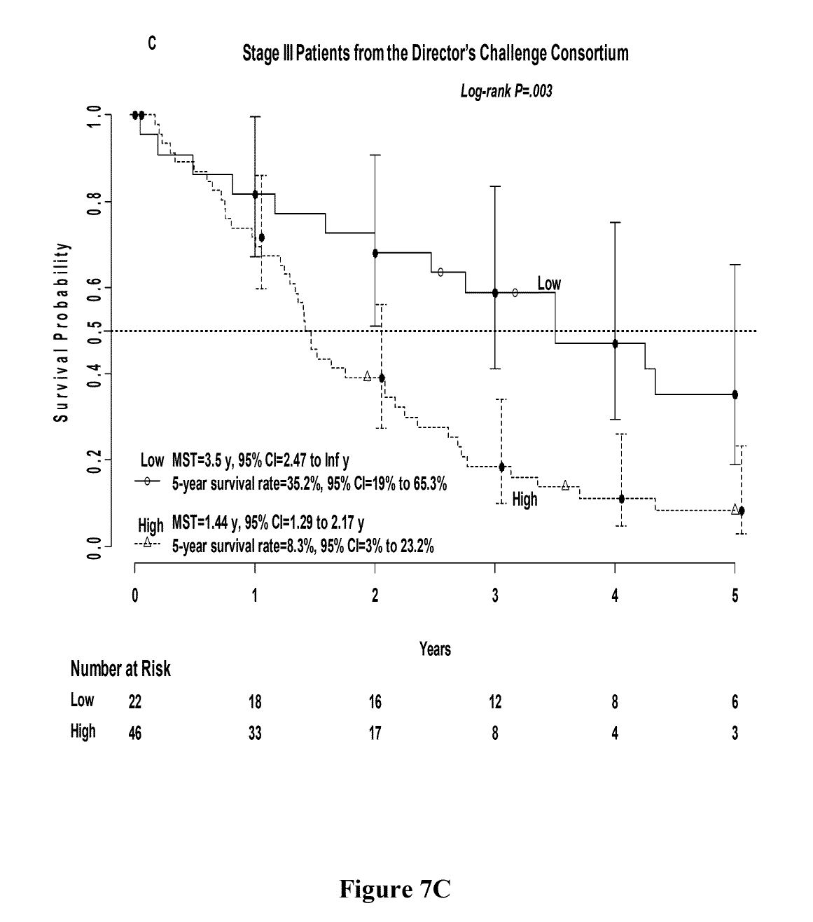

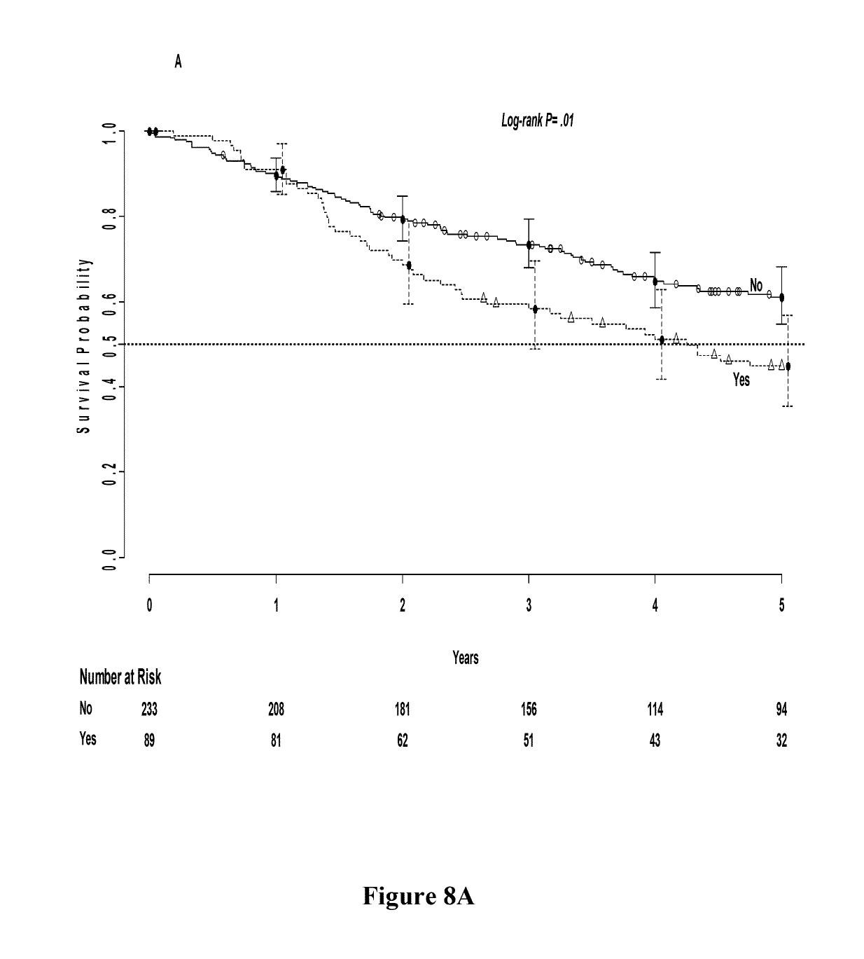

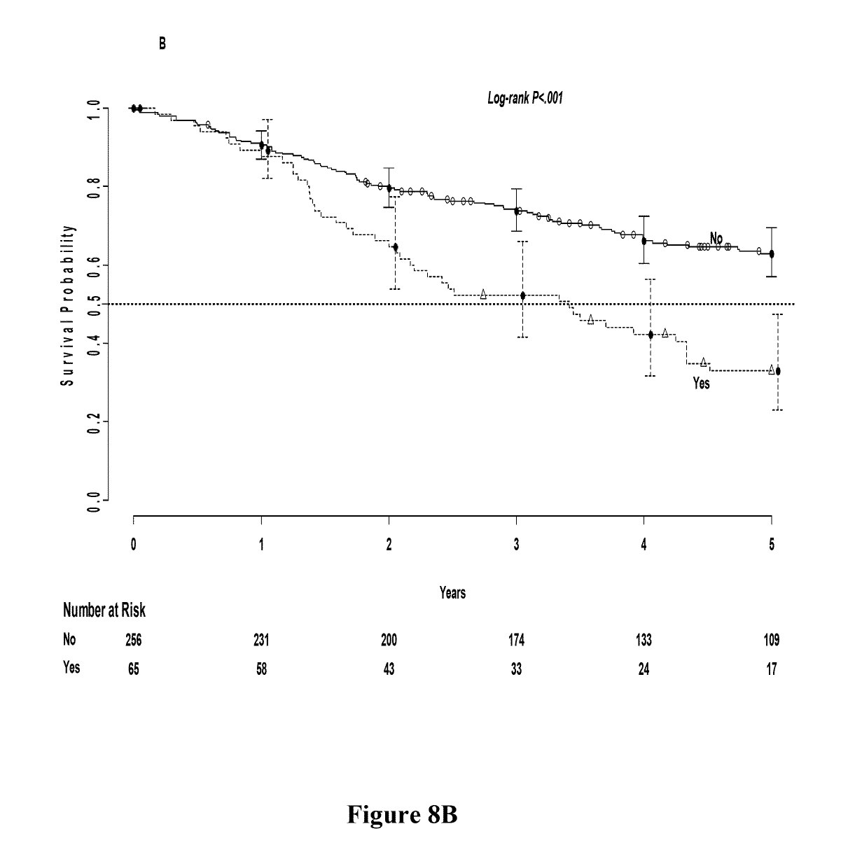

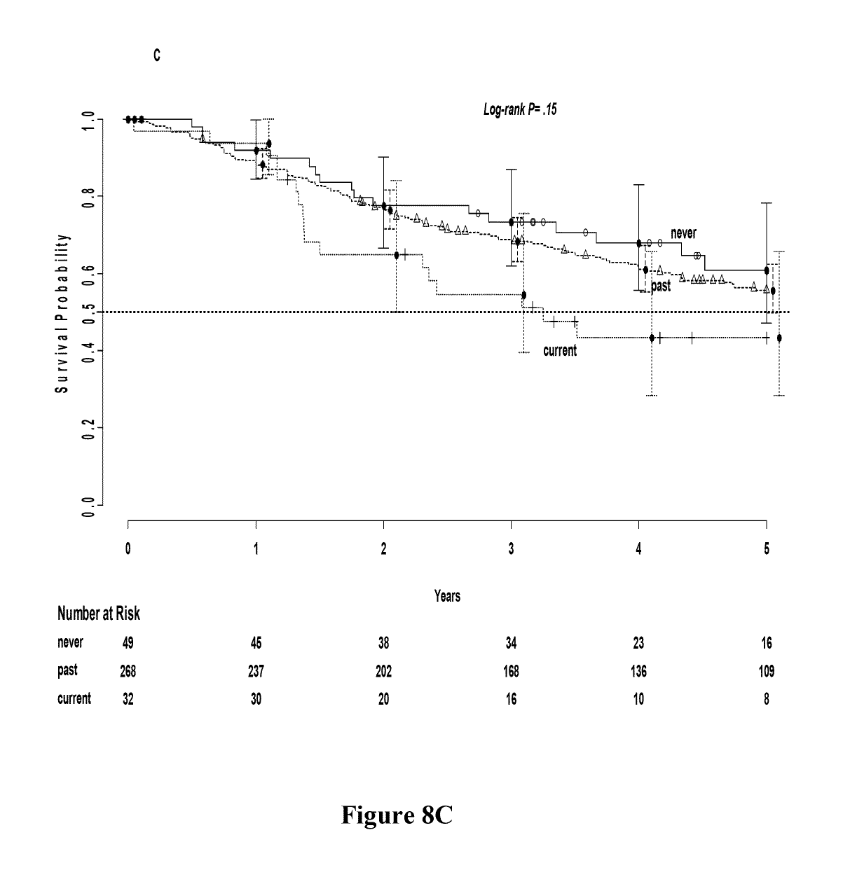

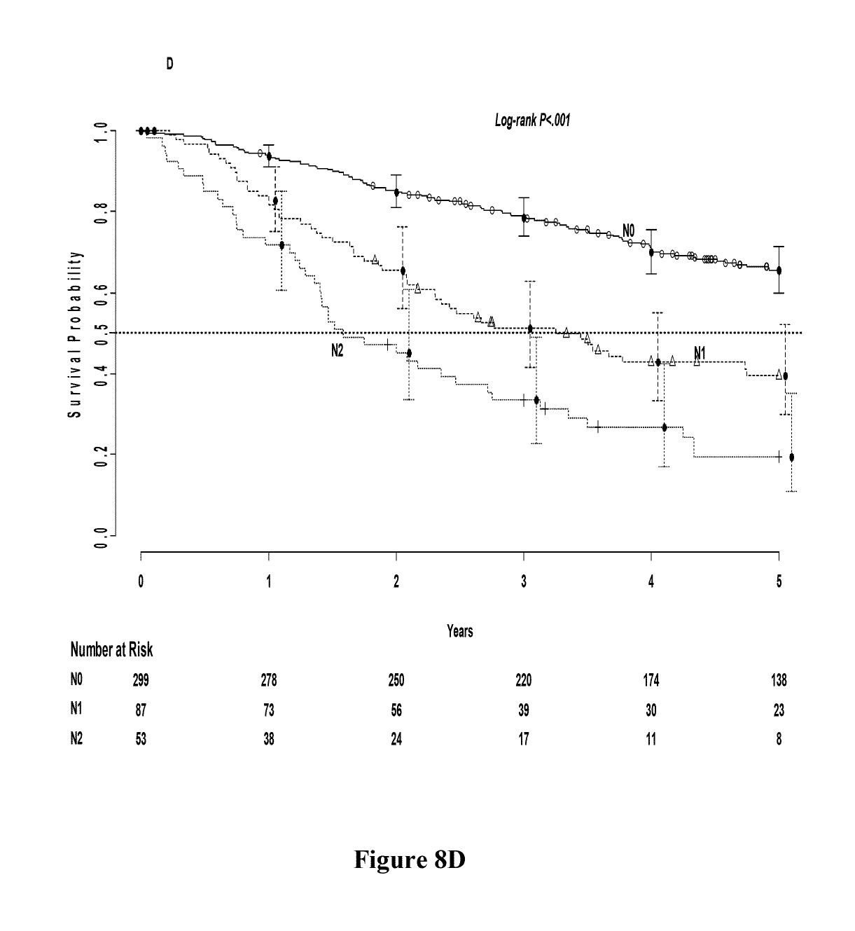

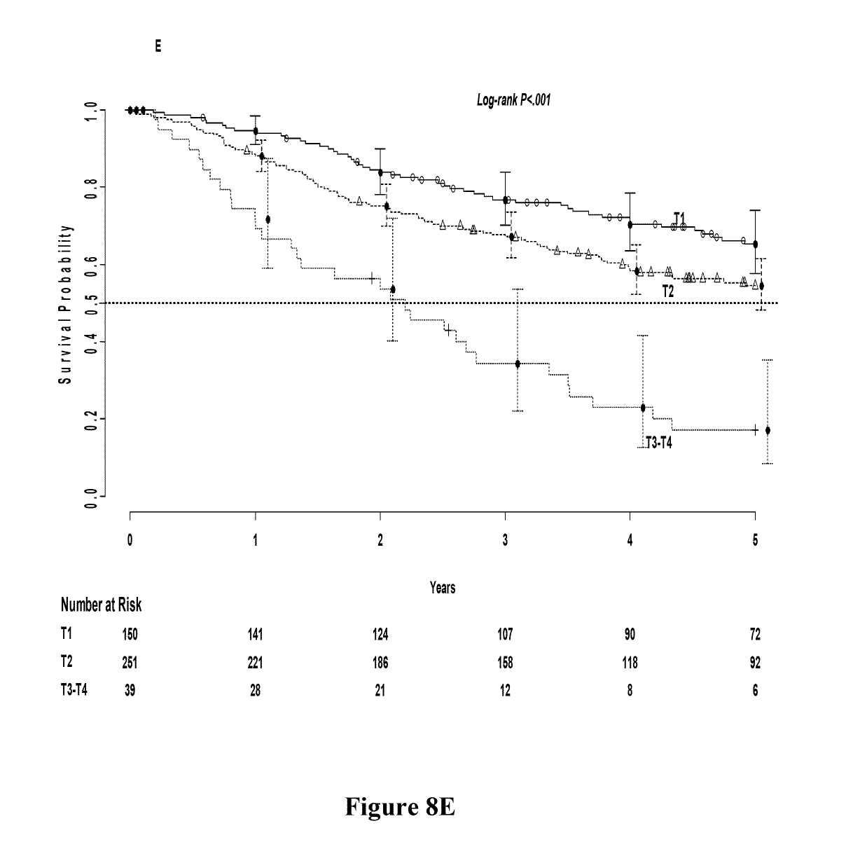

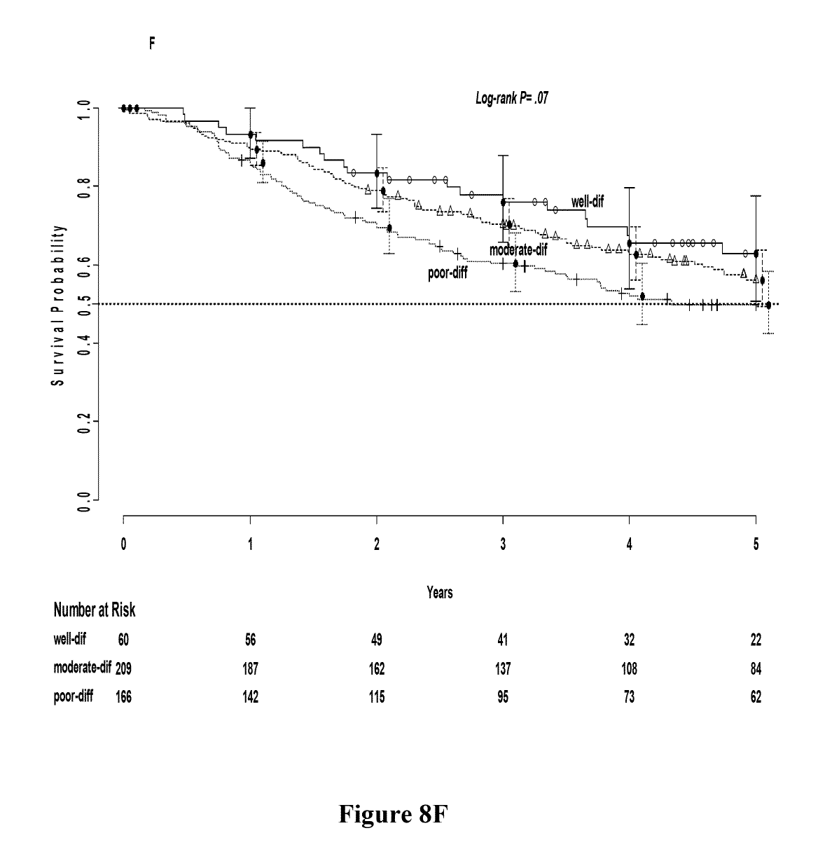

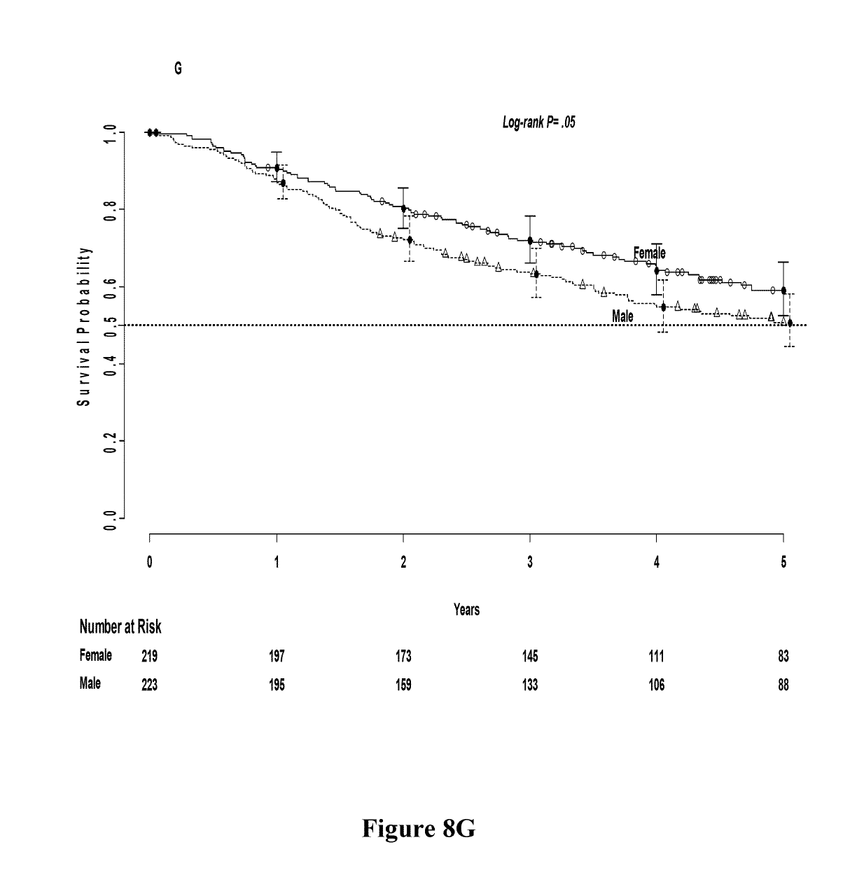

Several clinical predictors were statistically significantly associated with OS by log-rank test: TNM stage (P<0.001) (FIG. 7A), pathological T stage (P<0.001), pathological N stage (P<0.001), ACT (P=0.01), and adjuvant radiotherapy (P<0.001) (FIG. 8). For each clinical predictor, a statistically significant association of the malignancy-risk gene signature with OS was found in one or more risk groups: TNM stage IB and III ) P=0.004 and 0.003, respectively); pathological T stage T2 (P<0.001); pathological N stage N0, N1 and N2 (P=0.005, 0.03, and 0.004, respectively); moderately differentiated histologic grade (P<0.001); patients who did not receive ACT (P<0.001); patients who did not receive adjuvant radiotherapy (P<0.001); male and female patients (P=0.02 and <0.001, respectively); and former smokers (P<0.001) (FIG. 2). For example, TNM stage was associated with poor survival in patients with late stage disease (P<0.001) (FIG. 7A). For each TNM stage subgroup, patients with low malignancy-risk scores had increased OS compared with those with a high malignancy-risk score in stage IB and III (stage IB: log-rank P=0.004, HR=2.29, 95% CI=1.27 to 4.13; stage III: log-rank P=0.003, HR=2.57, 95% CI=1.36 to 4.86) (FIGS. 7B and C).

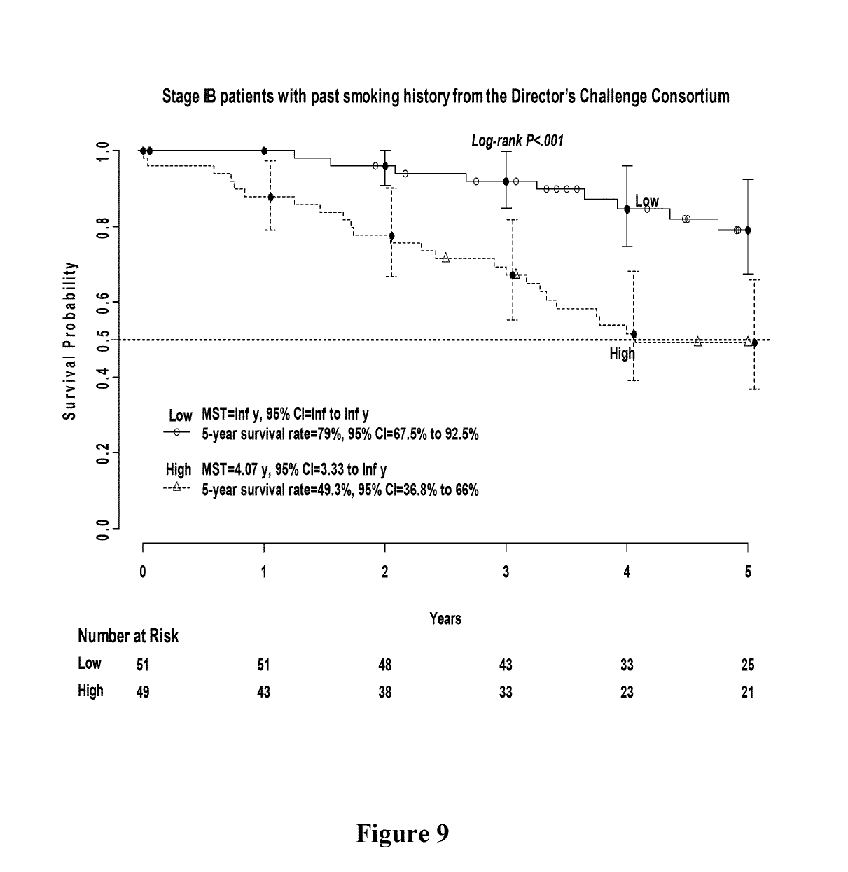

In addition, multivariable analysis using all clinical predictors (without the signature) yielded two statistically significant predictors of OS: TNM stage and smoking history. Because the malignancy-risk gene signature had shown a statistically significant association with OS in stage IB and III, smoking history was examined in the two subgroups to evaluate the usefulness of the malignancy-risk score within each subgroup (2 stages×3 smoking statuses). Subgroup analysis showed that for the stage IB patients with past smoking history, the malignancy-risk gene signature was able to differentiate the two risk groups, with increased OS observed in the group with a low malignancy-risk score (log-rank P<0.001; and HR=3.39, 95% CI=1.57 to 7.29) (FIG. 9). The 5-year survival rate estimate for the high malignancy-risk group (5-year survival rate=49.3%, 95% CI=36.8% to 66%) was less than that for the low malignancy-risk group (5-year survival rate=79%, 95% CI=67.5% to 92.5%), and their 95% confidence intervals did not overlap (FIG. 9)

The inventors also investigated whether interactions between a clinical predictor and the median-spilt malignancy-risk score existed. A statistically significant interaction between the malignancy-risk gene signature and TNM stage was observed after adjusting for other clinical predictors (stage IB HR=6.23, 95% CI=1.19 to 32.53, PInteraction=0.03 and stage III HR=6.94, 95% CI=1.27 to 38.07, PInteraction=0.03) (data not shown).

Univariate Analysis

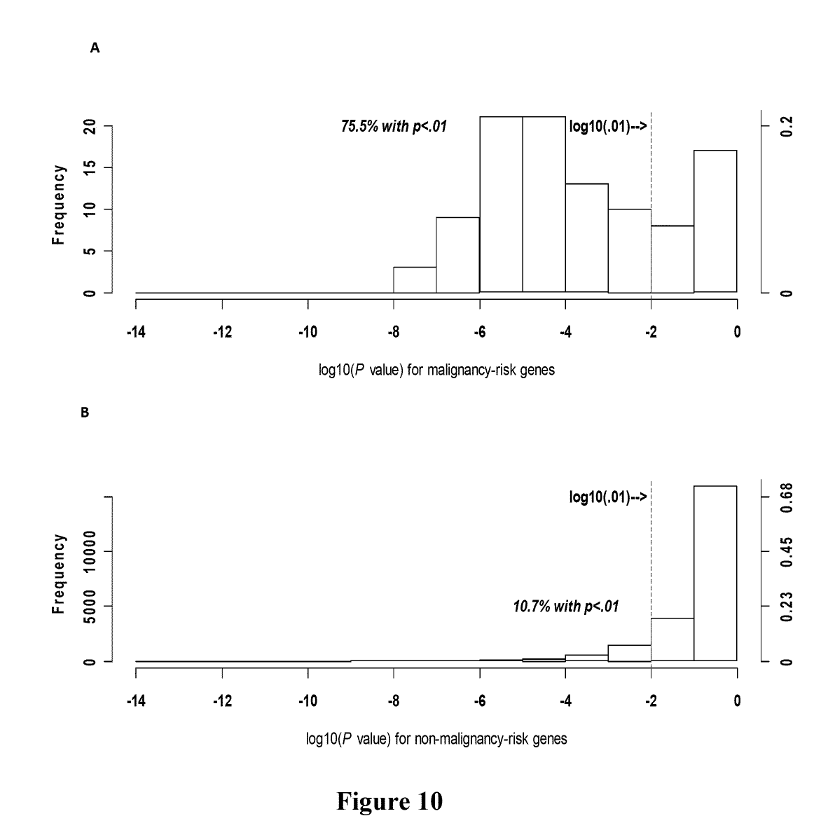

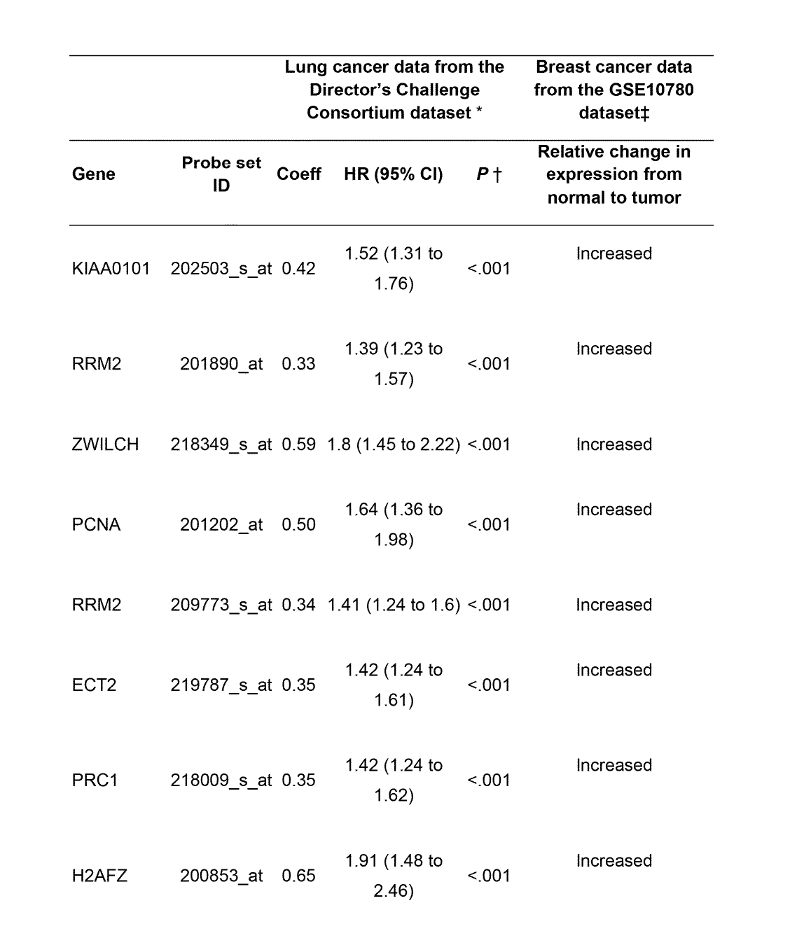

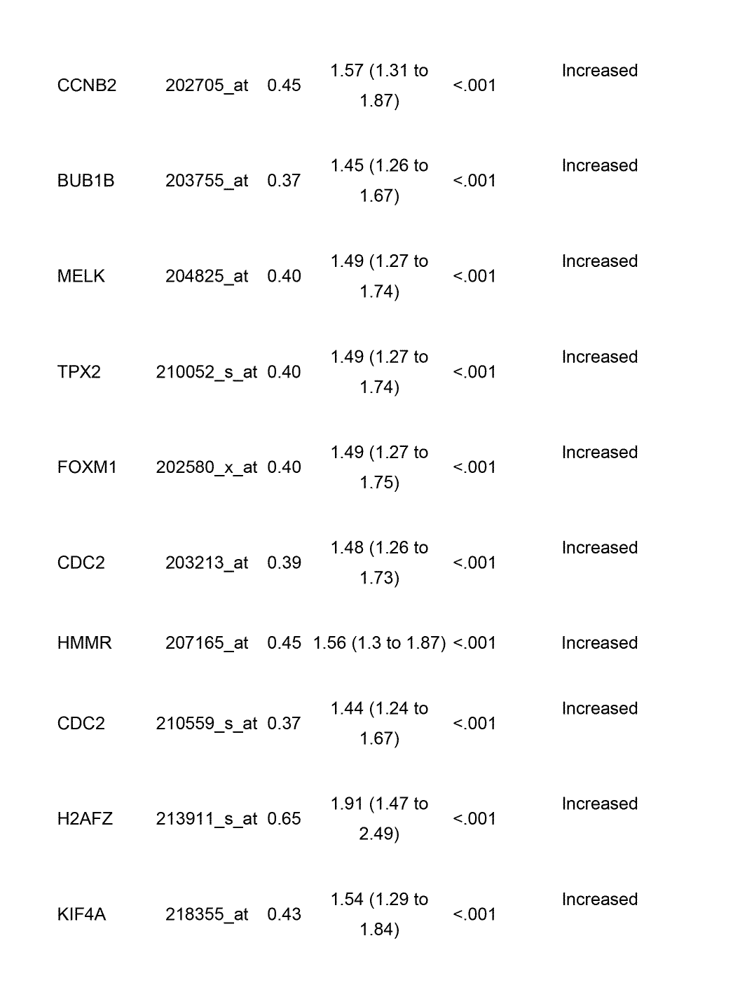

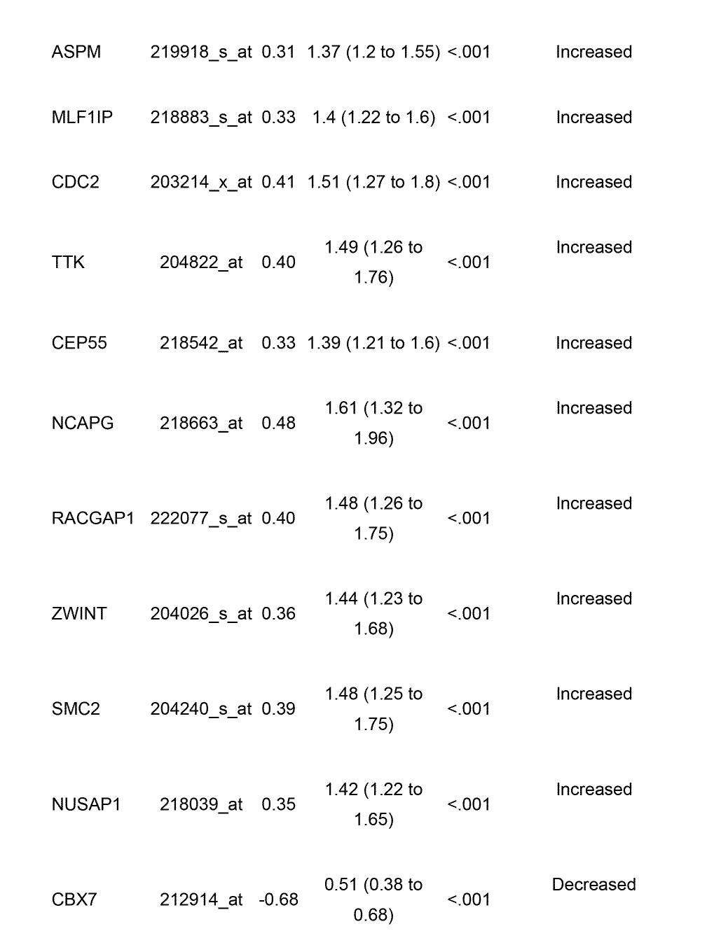

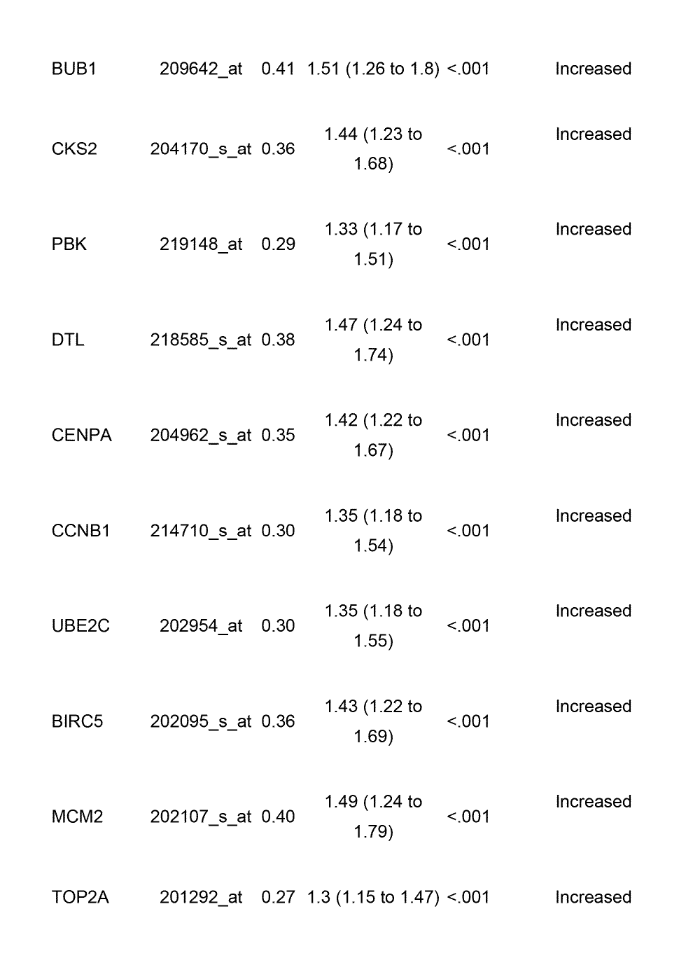

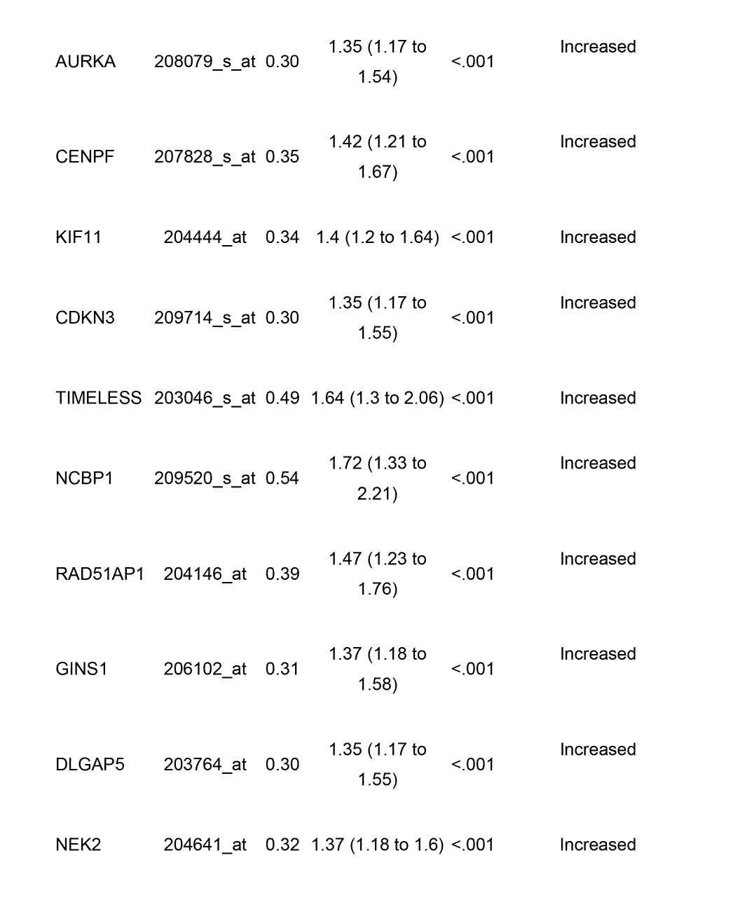

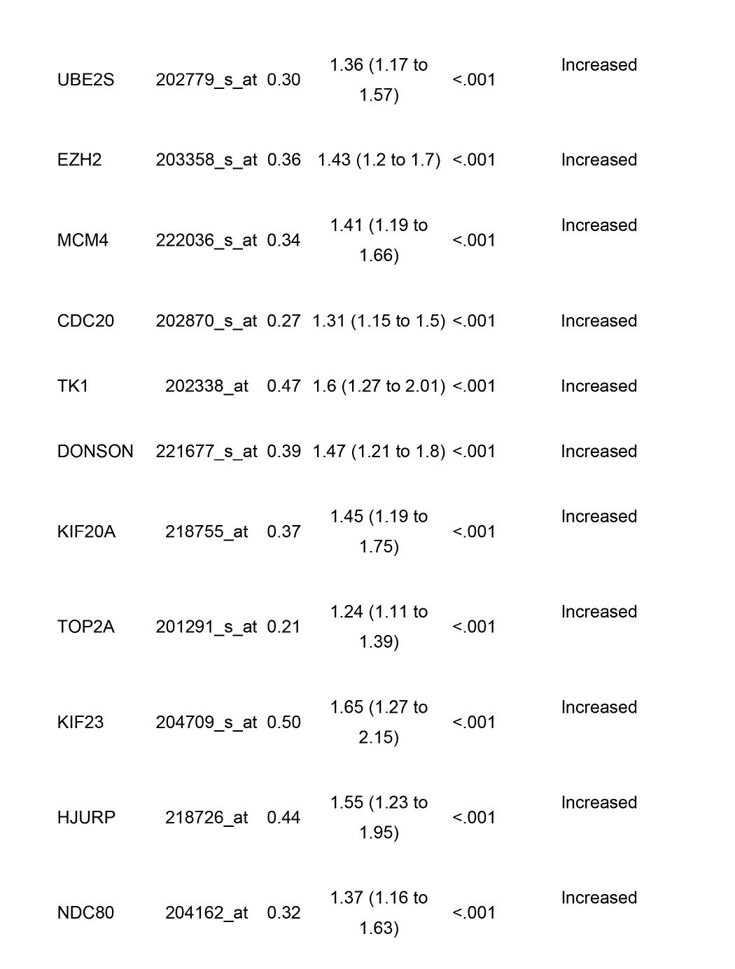

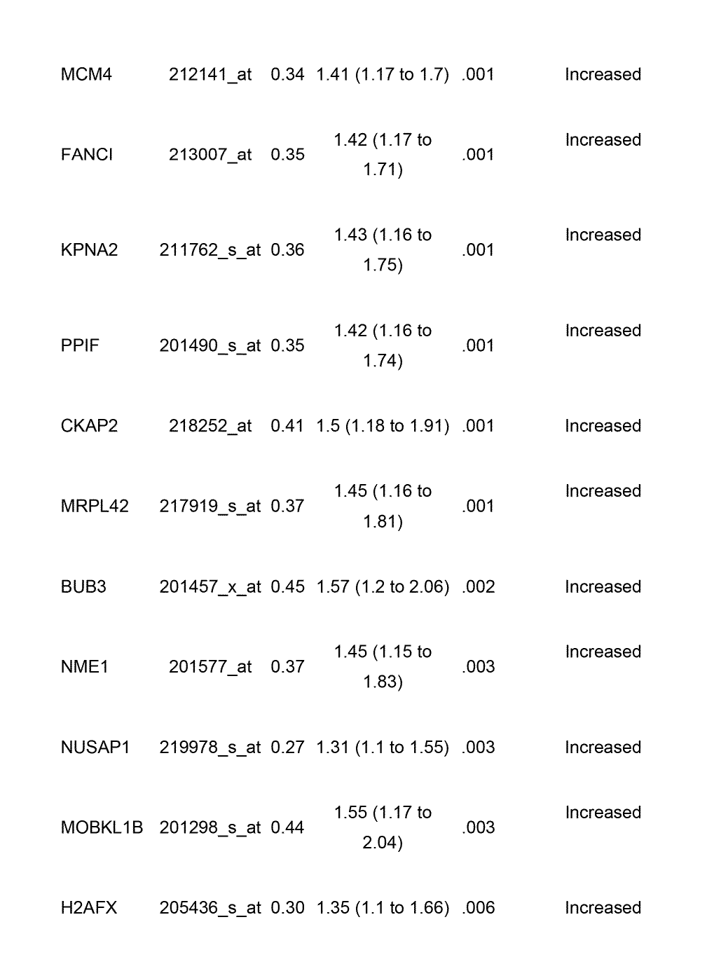

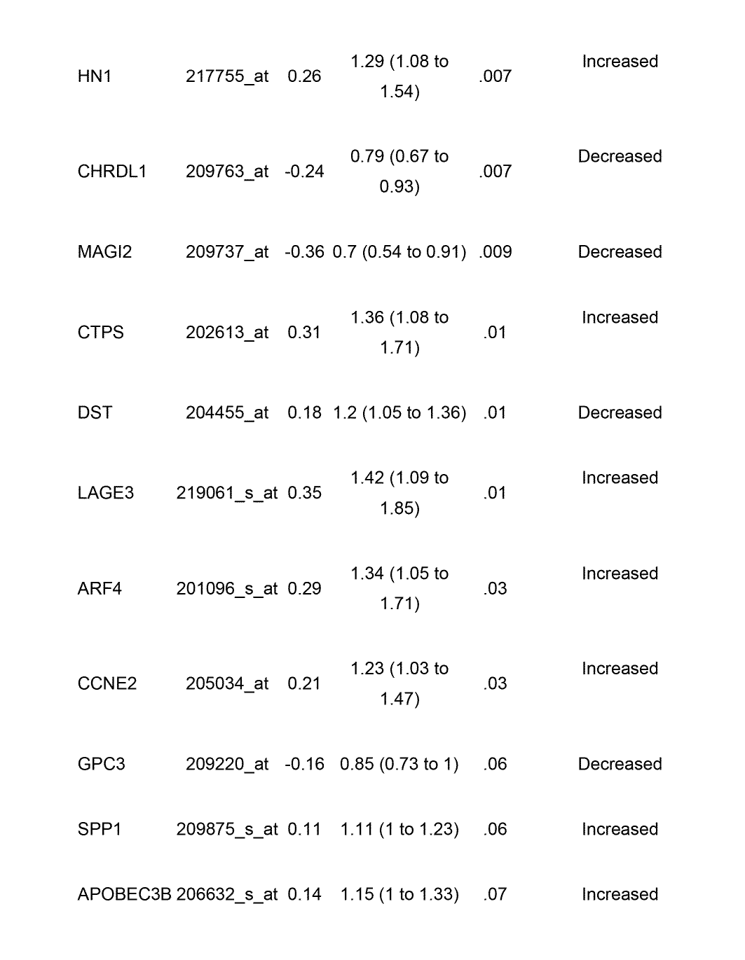

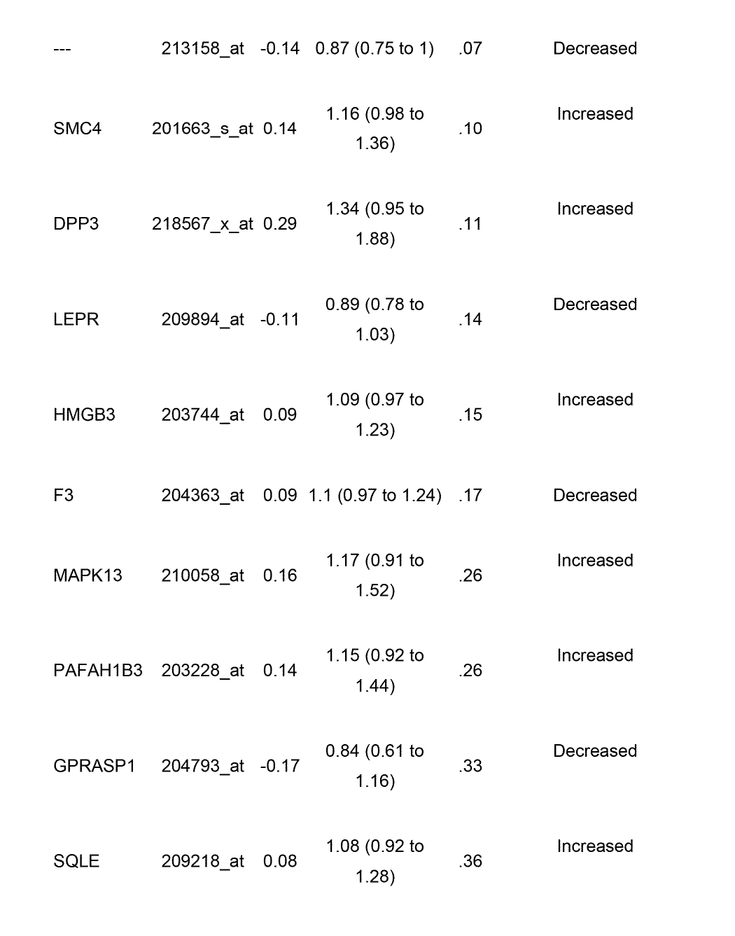

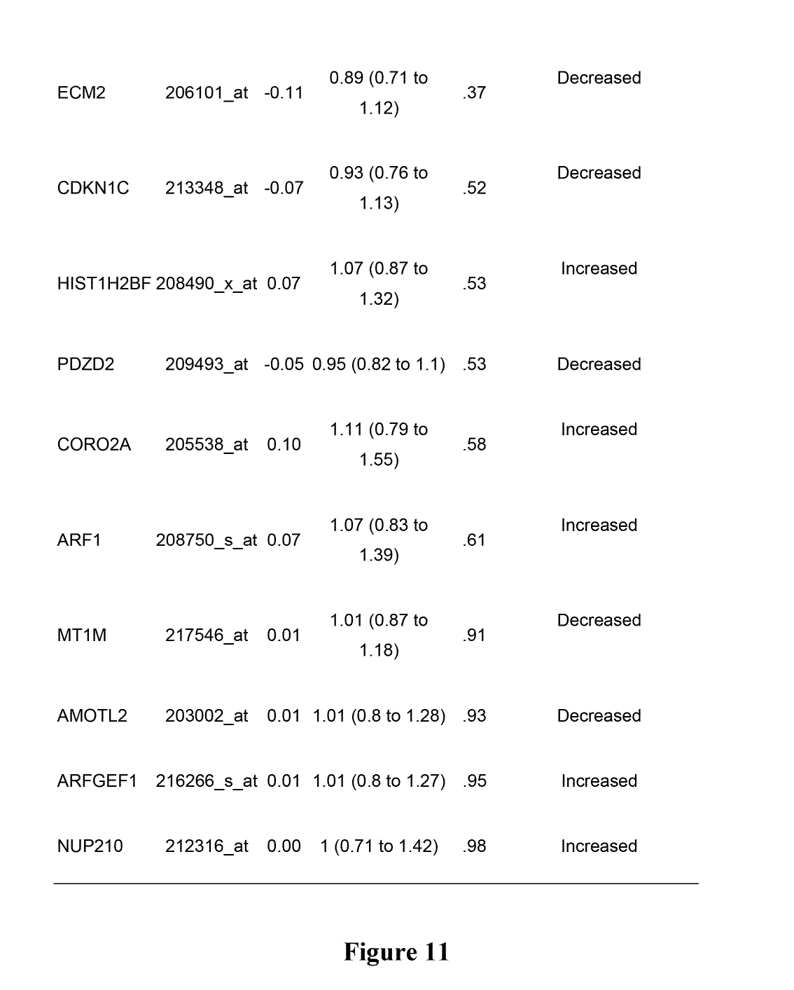

Univariate analysis by Cox proportional hazards modeling yielded 75.5% probe sets with statistically significant expression of the malignancy-risk genes (77 probe sets for 70 genes with P<0.01) in the Director's Challenge Consortium dataset. In contrast, there were only 10.7% probe sets with statistically significant expression of non-malignancy-risk genes. The difference between these two (75.5% vs 10.7%) was statistically significant (P<0.001 by Fishers exact test), indicating a strong association between the malignancy-risk gene signature and OS (FIG. 10). After adjusting for multiple testing at the 1% false discovery rate level there were 67 unique statistically significant malignancy-risk genes (74 probe sets), of which 48 genes (71.6%) are involved in cell proliferation (FIG. 11). All the 48 proliferative genes were correlated with shorter OS when the genes were over-expressed. Moreover, these genes were consistent with those identified in our previous study (Chen D T, Nasir A, Culhane A, et al. Proliferative genes dominate malignancy-risk gene signature in histologically-normal breast tissue. Breast cancer research and treatment 2010; 119(2):335-463 in which the malignancy-risk gene signature was identified in breast tumors (FIG. 11).

Prognostic and Predictive Value of the Malignancy-Risk Gene Signature for NSCLC

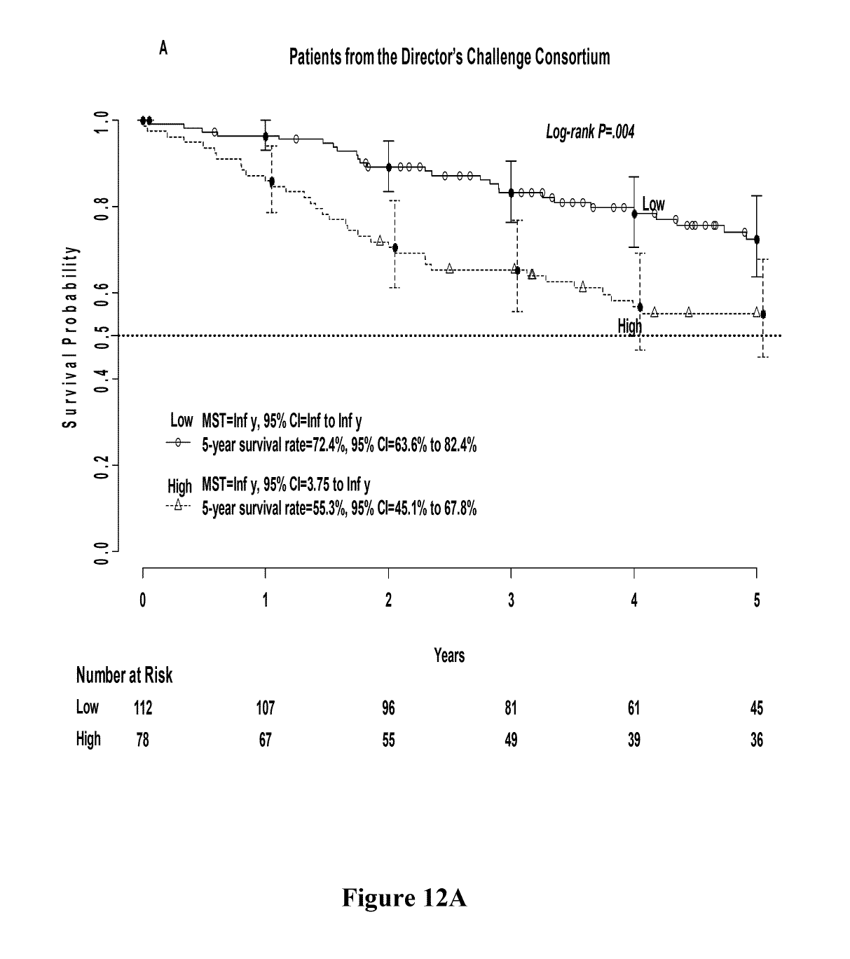

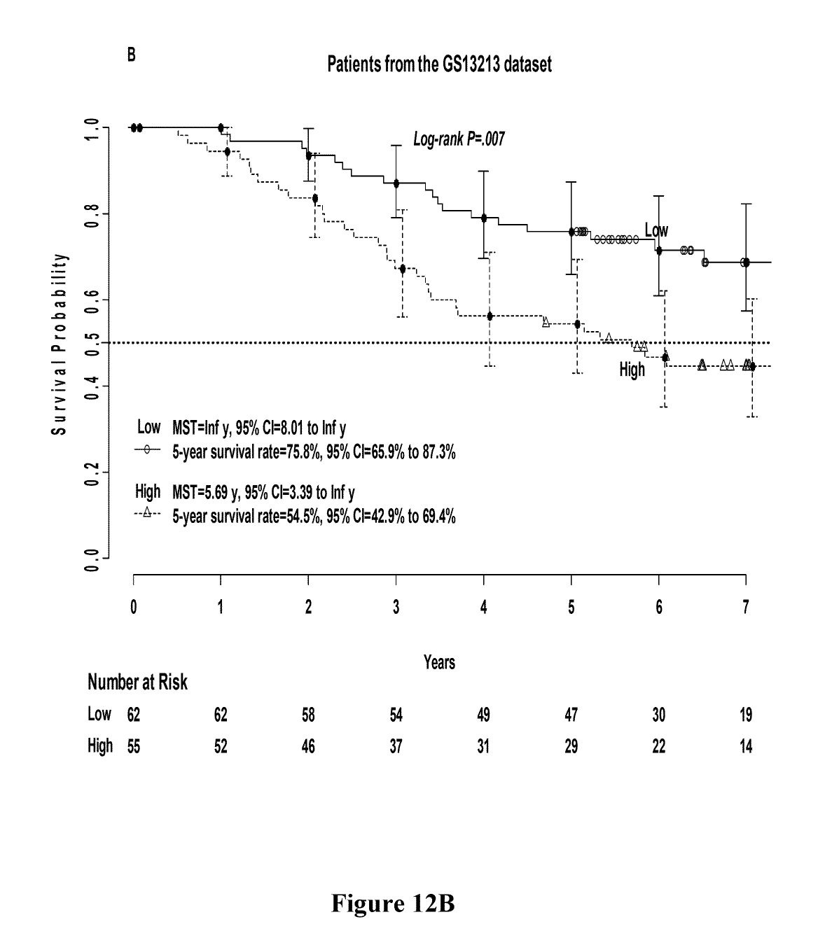

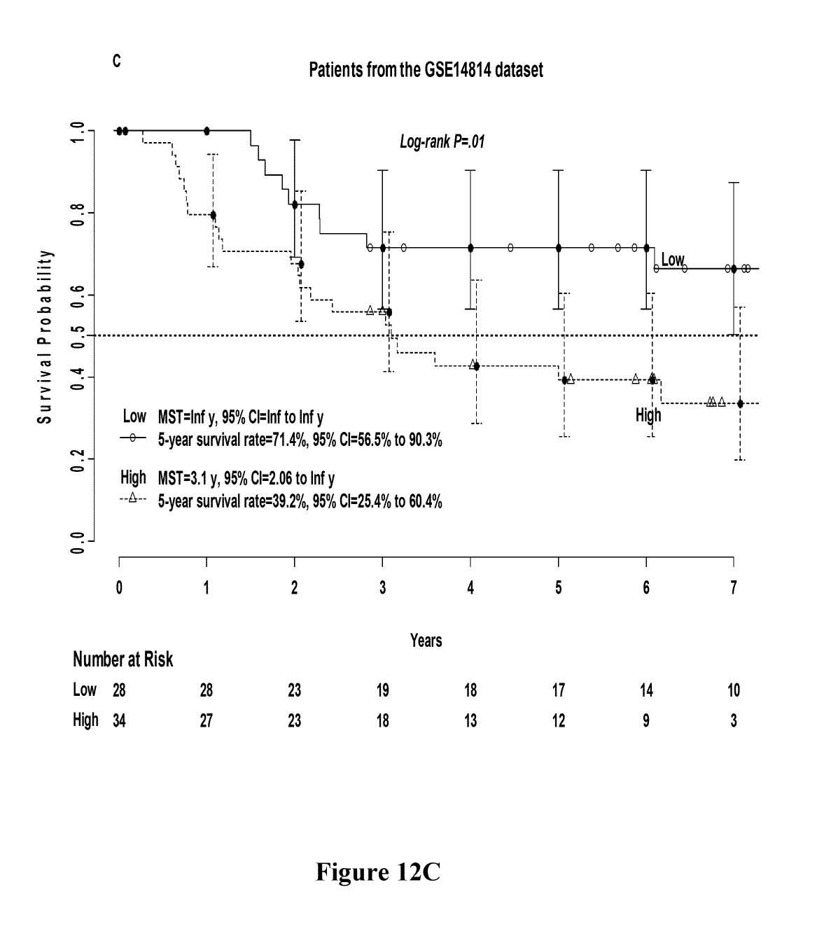

The malignancy-risk gene signature was prognostic for OS in the patients who did not receive ACT or RT with poorer survival in the high malignancy-risk group in the three lung cancer datasets (Director's Challenge Consortium dataset: log-rank P=0.004, and HR of death=2.10, 95% CI=1.26 to 3.51; GSE13213 dataset; log-rank P=0.007, and HR of death-32 2.17, 95% CI=1.22 to 3.86; GSE14814 dataset; log-rank P=0.01, and HR of death=2.57, 95% CI=1.17 to 5.64) (FIG. 12, A-C).

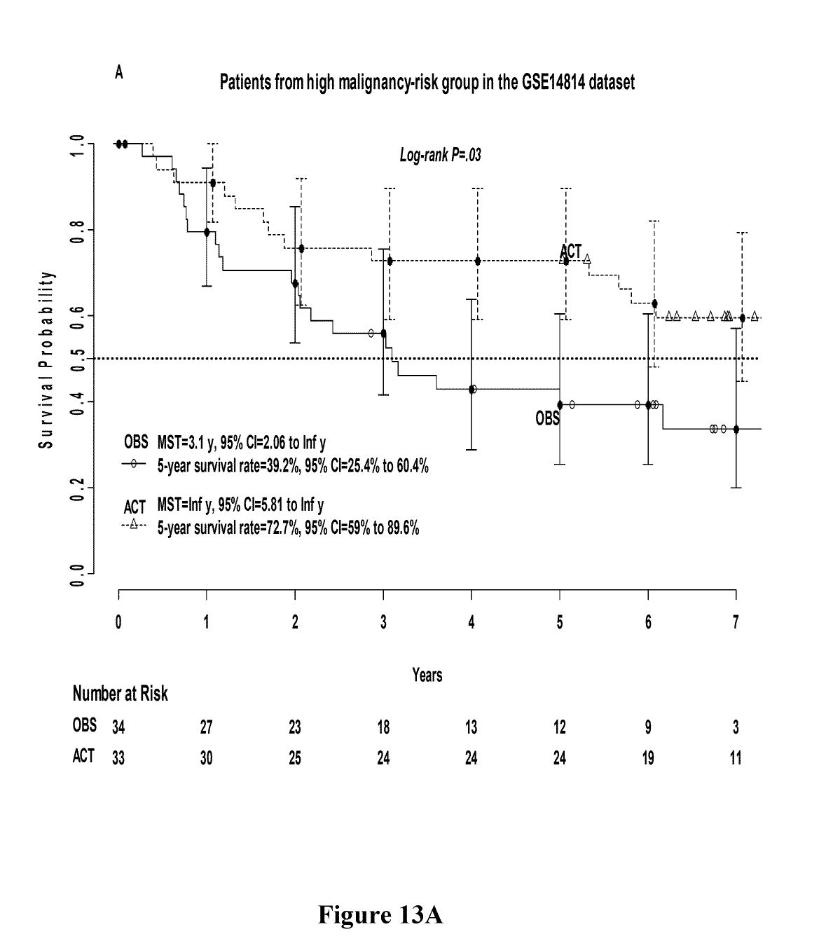

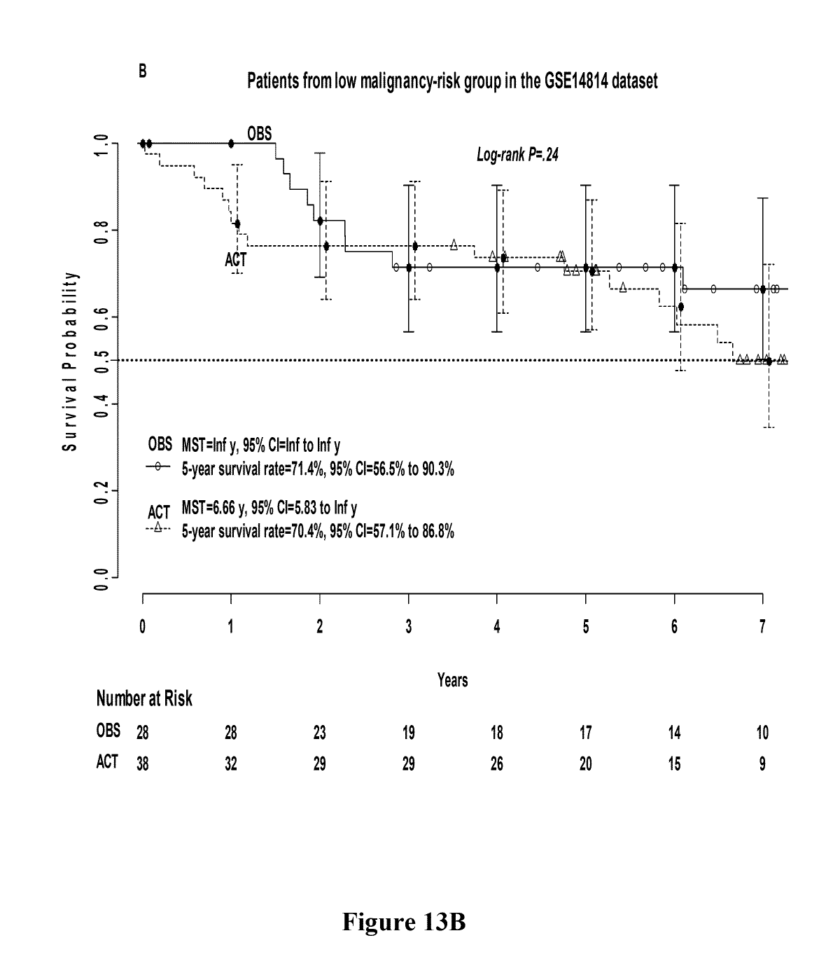

For the predictive value evaluated in the GSE14814 dataset, the ACT cohort experienced longer survival than the observation cohort in the high malignancy-risk group (log-rank P=0.03; and HR of survival=0.48, 95% CI=0.24 to 0.96) (FIG. 13A). Patients in the high malignancy-risk group had a higher 5-year survival rate estimate for patients who received ACT (5-year survival rate=72.7%, 95% CI=59% to 89.6%) compared with those who received observation, only (5-year survival rate=39.2%, 95% CI=25.4% to 60.4%) (FIG. 13A). In the low malignancy-risk group, patients who received ACT had a lower survival probability in the first two years than those who received observation; however, there was no statistically significant difference between the two groups (FIG. 13B). Moreover, the interaction between ACT and the malignancy-risk gene signature was statistically significant (HR=0.29, 95% CI=0.10 to 0.85, PInteraction=0.02).

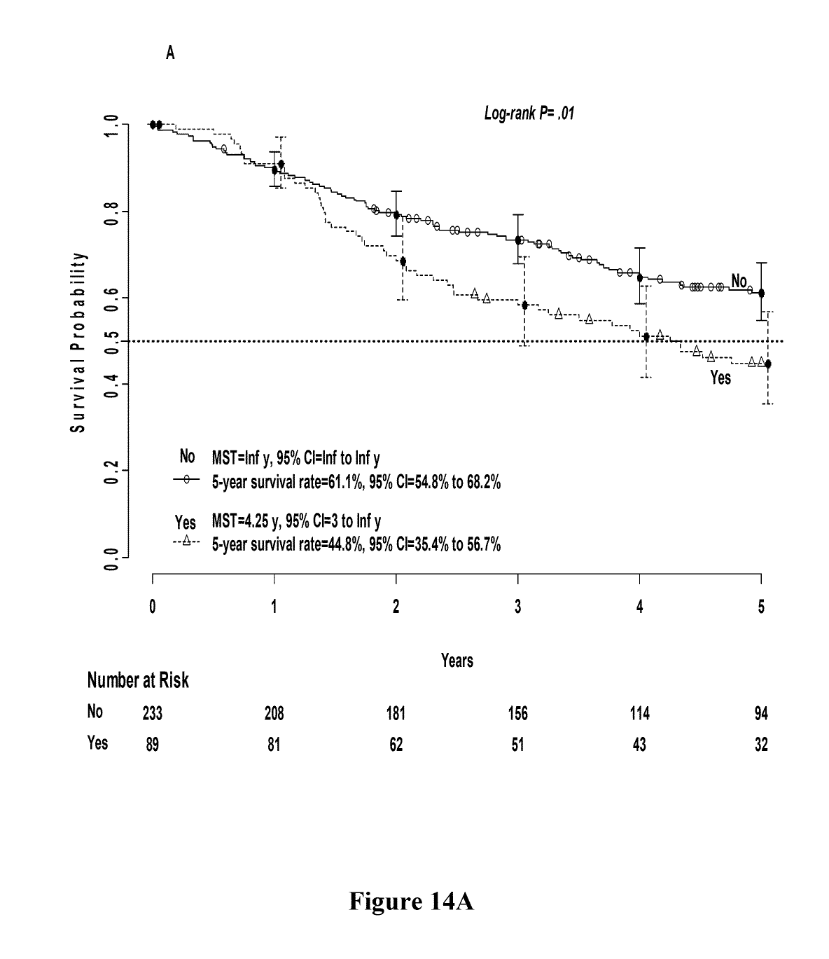

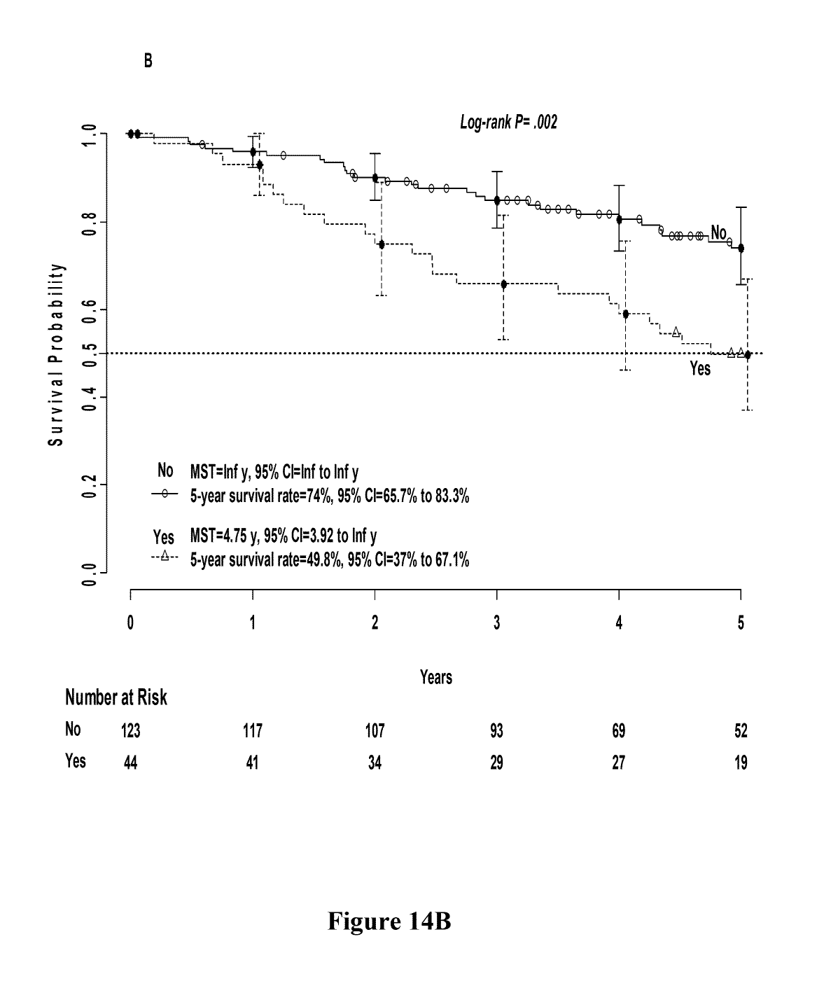

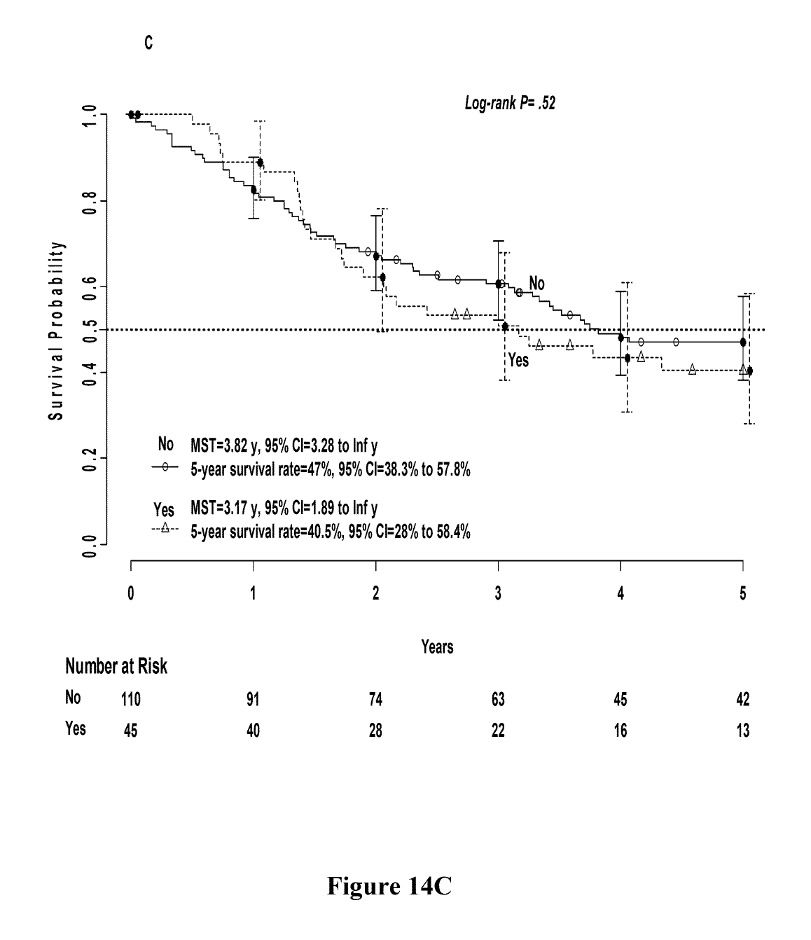

Evaluation of the predictive value in the Director's Challenge Consortium dataset showed that patients who received ACT had poorer OS in both the high and low malignancy-risk groups compared with patients who did not receive ACT. Because this was a retrospective study, patients receiving ACT had poorer OS than those who did not get ACT (log-rank P=0.01; and HR of death=1.59, 95% CI=1.12 to 2.27) (FIG. 14). It is likely that the patients receiving ACT had high-risk clinical characteristics such that ACT was recommended. As expected, the patients who received ACT had shorter survival than those who received non-adjuvant treatment in the low malignancy-risk group (log-rank P=0.002; and HR of death=2.36, 95% CI=1.34 to 4.15) (FIG. 14). However, this result should not be interpreted as indicating that ACT did harm to patients, but is indicative that poorer survival may be associated with high-risk clinical characteristics. On the other hand, the high malignancy-risk group also showed a poorer survival in the ACT cohort, whereas the HR was relatively small compared with that of the low malignancy-risk group (log-rank P=0.52; and HR of death=1.16, 95% CI=0.73 to 1.86) (FIG. 14). This observation indicates that there may be some clinical advantage to adjuvant treatment in the high malignancy-risk group, but the benefit could not overcome the detrimental contribution of high-risk clinical characteristics.

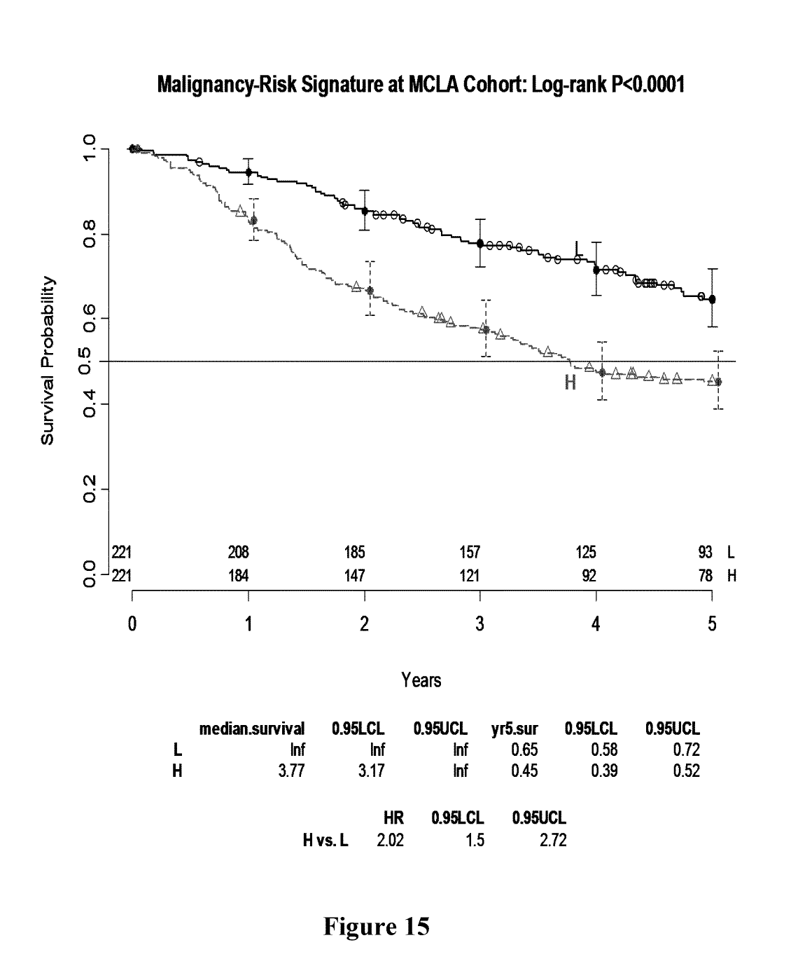

In early-stage NSCLC patients, the MR signature can predict overall survival (OS) and have prognostic and predictive effects: (1) the MR score was positively correlated with most MR genes (high MR score linking to high expression of MR genes; FIG. 15a) and demonstrated significant association with poor overall survival in MCLA cohort (HR=2.02; 95% CI=1.5-2.72; FIG. 15b).

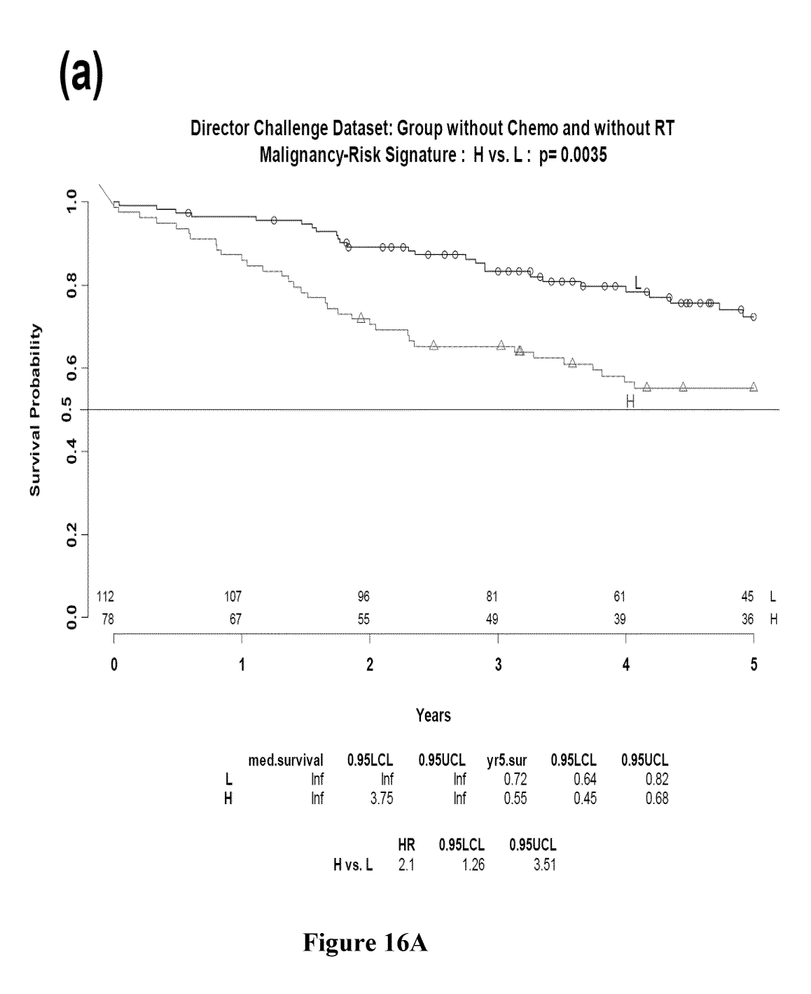

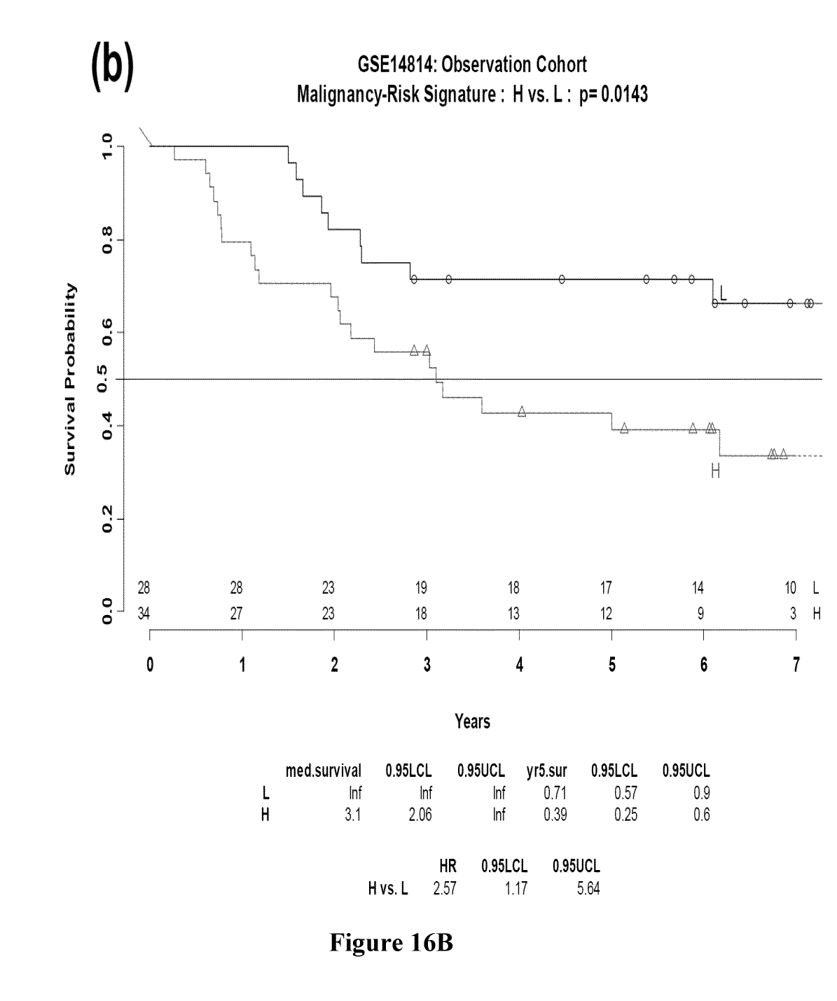

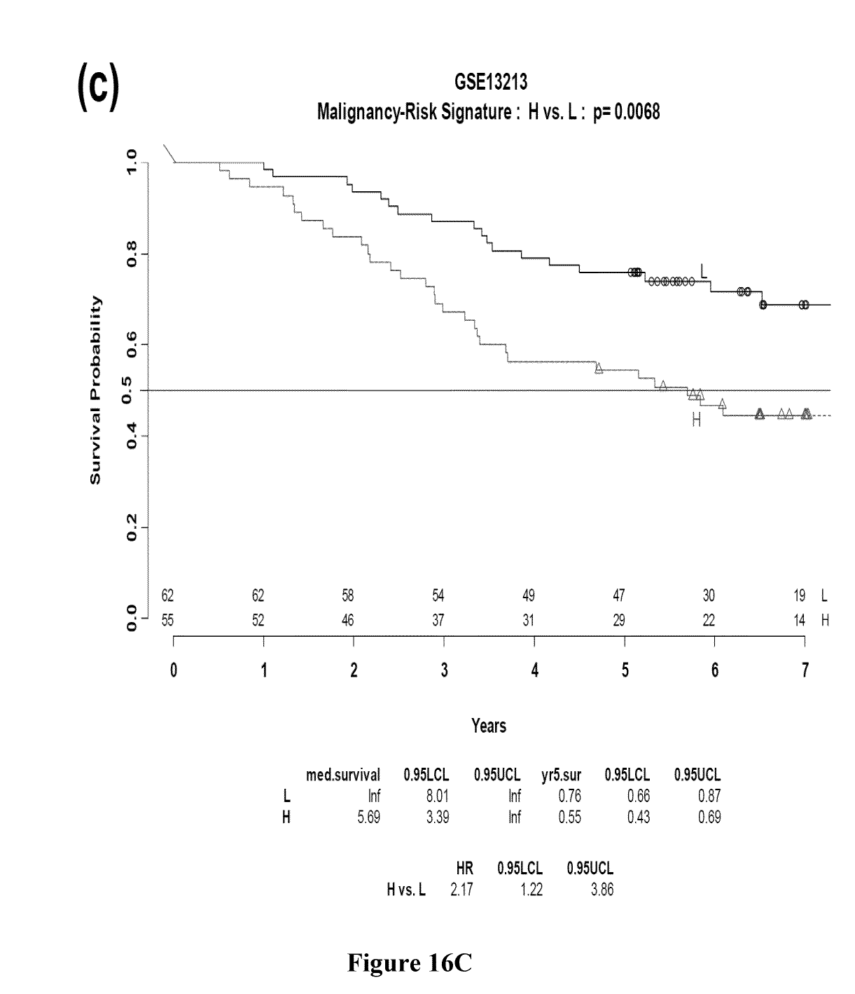

(2) By evaluating three NSCLC microarray datasets (MCLA, GSE13213, and GSE14814), the MR signature showed the prognostic feature and was able to predict OS in patients who did not receive adjuvant treatments in the three datasets (p=0.01-0.0035; FIG. 16). Patients with high MR score tended to have a shorter survival compared to the low score group (HR=2.1-2.57).

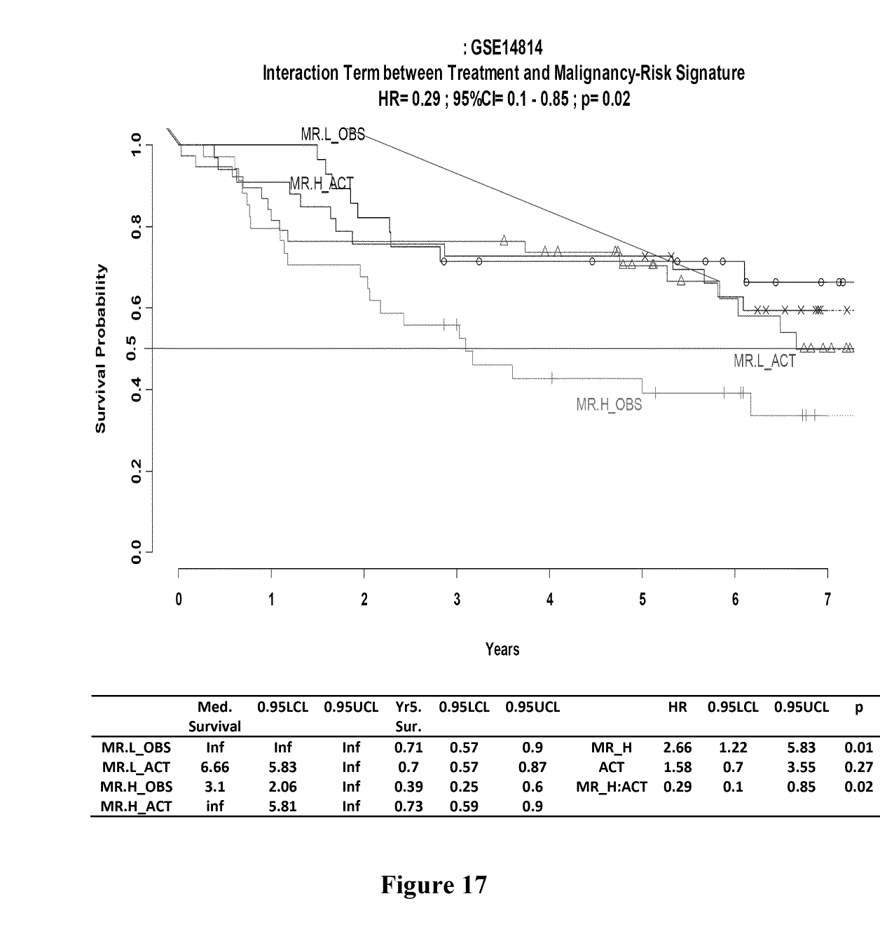

(3) The MR signature showed the predictive feature with a significant interaction effect between ACT and the signature (HR=0.29; p=0.02; FIG. 17a), suggesting the relationship between survival benefit and ACT was affected by the signature. Specifically, the survival benefit from ACT relative to the observation cohort was considerably greater in high MR score group (HR=0.48 with p=0.03; FIG. 17b) with 34% improvement in 5-year survival rate (73% versus 39%).

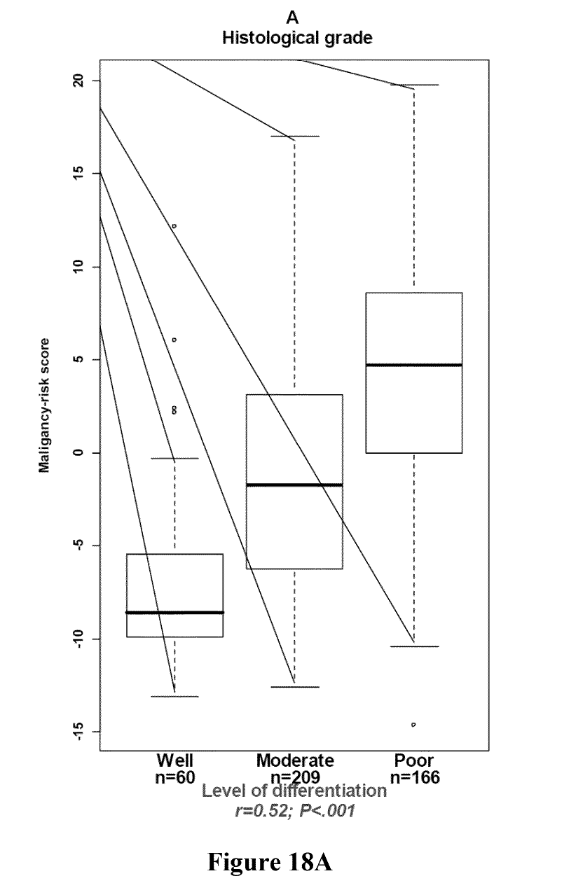

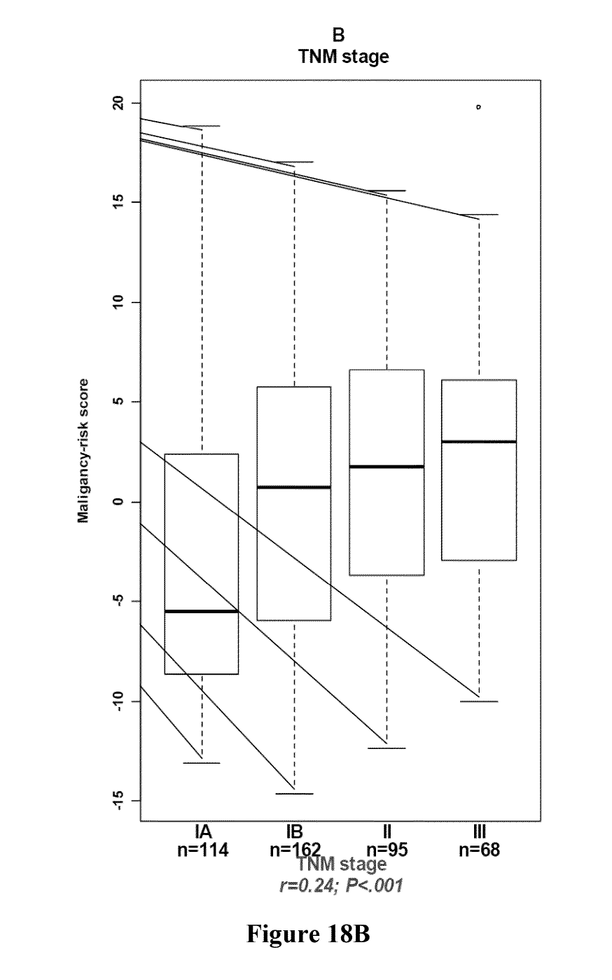

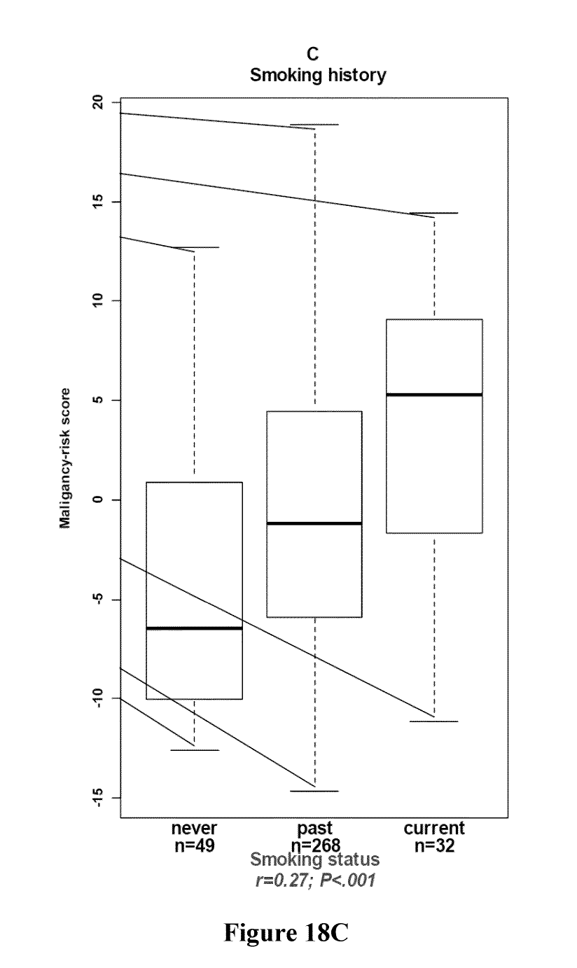

(4) The MR signature was further shown to have strong clinical associations with histologic grade, TNM stage, and smoking history in MCLA cohort (p<0.001; FIG. 18). Patients with low-grade and low stage tumors, small tumor size, no lymph node involvement, and those who never smoked, tended to have a low MR score.

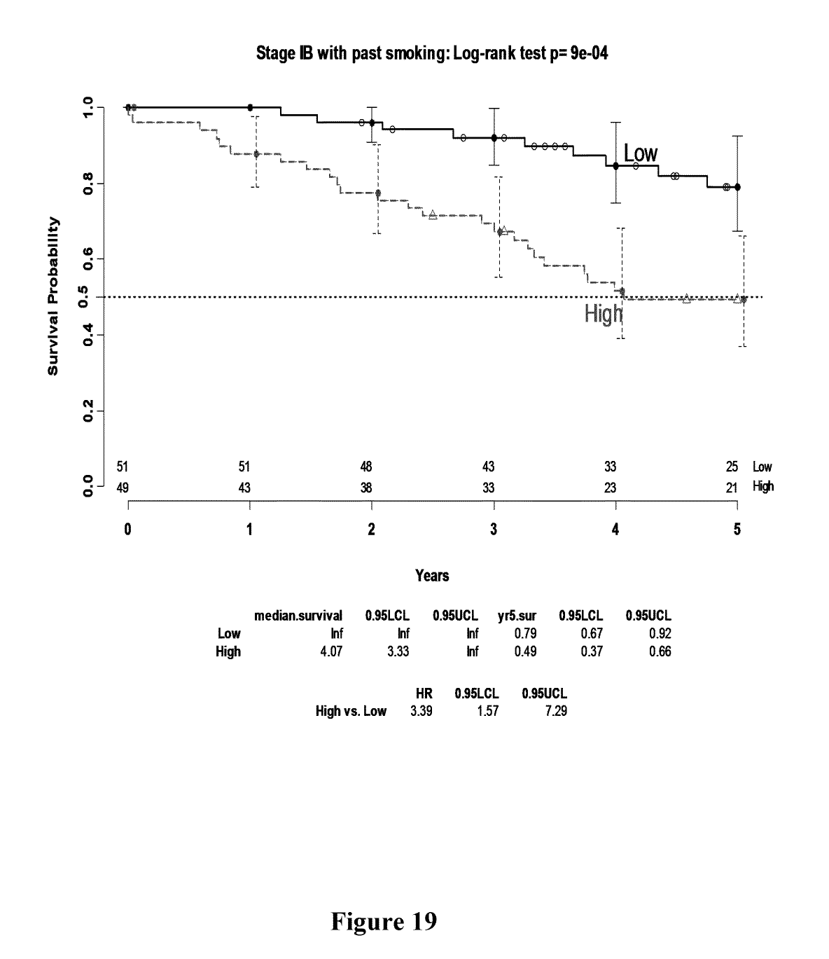

(5) Subgroup analysis showed that for the stage IB patients with past smoking history, the malignancy-risk gene signature was able to differentiate the two risk groups, with increased OS observed in the group with a low malignancy-risk score (log-rank P<0.001; HR=3.39; FIG. 19). Because the benefit of ACT remains nuclear in stage IB NSCLC, the signature may have potential clinical application for stage IB patients, such as recommendation of ACT only for stage IB patients with a high malignancy-risk score.

(6) The MR signature had a higher percent variability (44-50%) and a strong correlation of the loading coefficients (0.92-0.98) by the 1^{st} principal component in the three lung datasets, suggesting the 1st principal component well represents the MR signature.

(7) Many over-expressed MR genes in breast cancer were associated with poorer survival in the lung cancer by univariate analysis (70% with p<0.05).

(8) The MR signature showed clinical association with cancer relapse/progression, and prognosis in the breast cancer. Similarly, the ME signature demonstrated significant association with OS and other clinical predictors in the lung cancer, such as TNM stage, and histologic grade.

Association with Drug Sensitivity

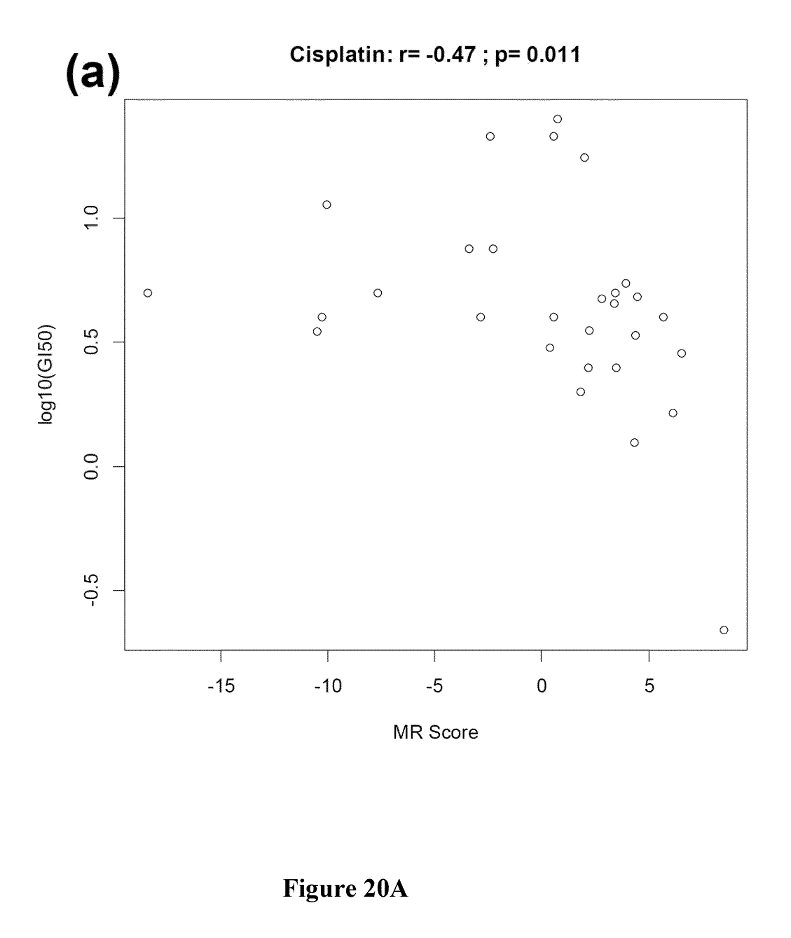

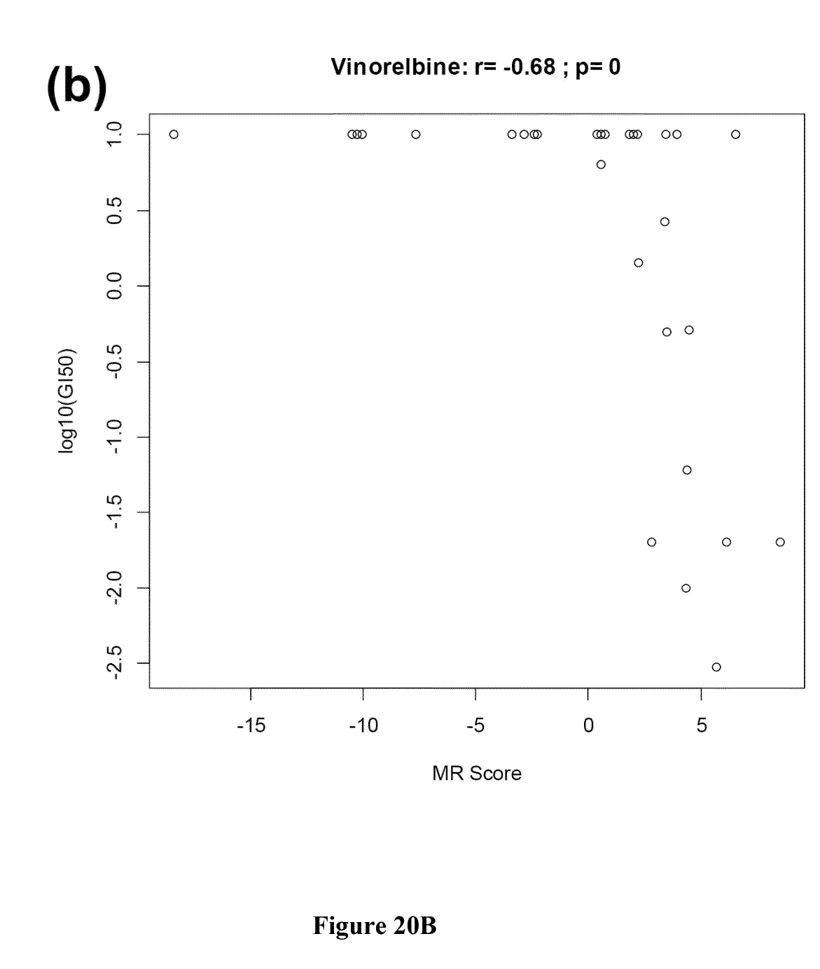

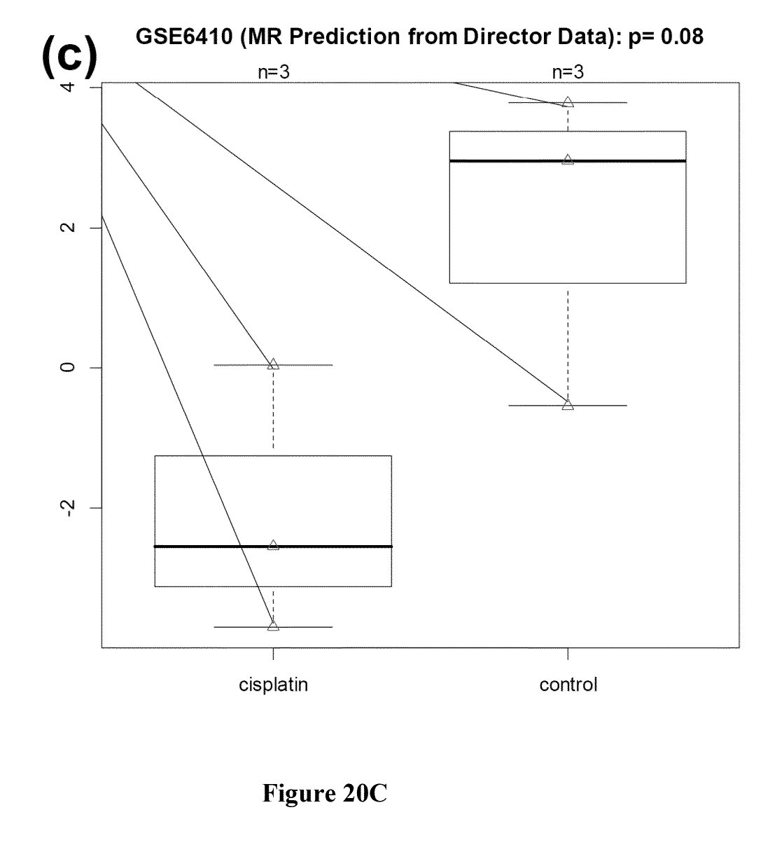

The inventors have identified 10 published datasets describing drug sensitivity in cell lines. Three of them have been analyzed to show the potential of the MR signature for its association with drug sensitivity. In Gemma et al.'s study (GSE4127) which examined anticancer drugs in lung cancer using gene expression data (Gemma, A, C Li, Y Sugiyama, K Matsuda, Y Seike, S Kosaihira, Y Minegishi, R Noro, M Nara, M Seike, A Yoshimura, A Shiomoya, A Kawakami, N Ogawa, H Uesaka, and S Kudoh, Anticancer drug clustering in lung cancer based on gene expression profiles and sensitivity database. BMC cancer, 2006, 6: p. 174), the MR signature showed negative correlation with Cisplatin and vinorelbine (FIG. 20a-b). A high MR score tends to be associated with low GI50 (sensitivity) in both drugs. In addition, in the Almeida et al.'s study (GSE6410), the MR score was lower in the cisplatin treated group than in the control group in A489 cell line (FIG. 20c).

Moreover, the inventors have previously demonstrated the better survival benefit from ACT compared to the observation cohort in high MR score group in the Zhu et al.'s study (GSE14818) which patients in the ACT cohort received cisplatin plus vinorelbine (FIG. 17). These results suggest the MR signature is a cisplatin sensitive signature and associates with the drug effect.

Taken together the data suggest: (a) the MR signature is a prognostic and predictive signature for early-stage NSCLC, (b) the signature shares similar biological and clinical traits between breast and lung cancer (transferability), and (c) the MR signature may govern cancer development at the early stage and associate with ACT.

Previously, the malignancy-risk (MR) gene signature having a dramatic enrichment of proliferative genes was derived from benign, but molecularly-abnormal breast tissue, suggesting proliferation may dominate the earliest stages of tumor development. Here, the malignancy-risk signature was applied to a large database in the Director's Challenge Consortium for the Molecular Classification of Lung Adenocarcinoma to evaluate whether the MR signature is a prognostic signature for overall survival in early-stage NSCLC. This MR gene profile, tested with a PCA scoring method, discriminated overall survival in lung cancer patients and was a predictor independent of pathological staging and other clinical parameters.

The MR signature was shown its clinical association with histologic grade, TNM stage, pathologic T and N staging, and smoking history (p<0.01). The MR score increases as the clinical characteristic becomes worse. Patients having the following characteristics: (1) low-grade; (2) low stage; (3) small tumor size; (4) no lymph node; or (5) never smoking tend to have a low MR score. In contrast, a high MR score occurs in patients with (1) high-grade; (2) high-stage; (3) large tumor size; (4) regional lymph node; or (5) currently smoking. When the MR signature was incorporated with each clinical parameter, it often showed the best survival in the low MR group with good clinical status (e.g., T1 group) and the worst survival in the high MR group with poor clinical status (e.g., T3-T4). Moreover, the low MR group had a better survival than the high MR group at each sub clinical group with a p<0.05 in several subgroups with (IB, III, T2, N0, N2, moderate-differentiation, and past smoking).

Univariate analysis showed an unexpected high proportion of significant MR genes associated with OS (75% compared to 19% in the whole genes with p<0.0001). Moreover, most of the significant genes showed the same concordance in breast cancer and in the lung cancer. When a MR gene showed over-expression in breast tumor, it also yielded a poor survival in lung cancer. These observations indicate that proliferation may govern not only breast cancer, but also attribute lung cancer development at the early stage. Two top genes, H2AFZ and ribonucleotide reductase subunit-2 (RRM2), showed a poor survival when they were over-expressed. H2AFZ is a histone variant and has been observed over-expression in breast cancer and colorectal cancer, suggesting its role in carcinogenesis and the malignancy of tumors (Svotelis, et al., H2A,Z overexpression promotes cellular proliferation of breast cancer cells. Cell Cycle, Jan 15; 9(2):364-370). RRM2, a rate-limiting enzyme in cell replication, has been shown to be associated with hepatocellular carcinoma (Satow, et al., Combined functional genome survey of therapeutic targets for hepatocellular carcinoma. Clin Cancer Res. May 1; 16(9):2518-2528), lung adenocarcinoma (MacDermed, et al., MUC1-associated proliferation signature predicts outcomes in lung adenocarcinoma patients BMC Med Genomics, 3:16), glioblastoma (Grunda, et al., Rationally designed pharmacogenomic treatment using concurrent capecitabine and radiotherapy for glioblastoma; gene expression profiles associated with outcome. Clin Cancer Res. May 15; 16(10):2890-2898), and colorectal cancer (Grade, et al, A genomic strategy for the functional validation of colorectal cancer genes identifies potential therapeutic targets. Int J Cancer, May 12) and has been considered as potential therapeutic target.

It is rare for a gene signature to have been successfully tested and validated in various independent datasets. The MR signature is one of a few to do so and has shown many unique biological and clinical features in the lung cancer; (a) significant association with overall survival, stage, grade, and other clinical variables; (b) prognostic and predictive effects on early-stage NSCLC which could provide additional advantage to help identify a subset patients at high-risk of death who may benefit from ACT; (c) majority of MR genes involved in proliferation which could help better understand the universal loss of cell cycle control in the earliest stages of tumor development.

A high MR signature clearly identifies aggressive tumors. The MR signature is a prognostic and predictive signature that could be used to optimize the potential benefits of ACT and minimize unnecessary treatment and associated morbidity. The MR signature can be used to predict response to specific chemotherapeutic regiments. The MR can also be used to direct not only a yes or no on ACT, but also indicate which ACT option might be optimal.

As detailed above, the inventors have demonstrated the malignancy-risk (MR) gene signature has prognostic and predictive elects in NSCLC patients and has great potential to characterize NSCLC at the molecular level. To move the signature forward as a personalized medicine strategy to aid clinical decision making, it is imperative to identify whether the MR signature correlates with response to specific chemotherapies such that the best therapy could be used to target individual patients. Another important step is to validate the signature in an independent large dataset, larger or comparable to the Molecular Classification of Lung Adenocarcinoma (MCLA) cohort to advance the signature to the next level of the analytical and clinical validity. (Shedden, K, J M Taylor, S A Enkemann, M S Tsao, T J Yeatman, W L Gerald, S Eschrich, I Jurisica, T J Giordano, D E Misek, A C Chang, C Q Zhu, D Strumpf, S Hanash, F A Shepherd, K Ding, L Seymour, K Naoki, N Pennell, B Weir, R Verhaak, C Ladd-Acosta, T Golub, M Gruidl, A Sharma, J Szoke, M Zakowski, V Rusch, M Kris, A Viale, N Motoi, W Travis, B Conley, V E Seshan, M Meyerson, R Kuick, K K Dobbin, T Lively, J W Jacobson, and D G Beer, Gene expression-based survival prediction in lung adenocarcinoma: a multi-site, blinded validation study. Nat Med, 2008, 14(8): p. 822-7)

Validating the MR Gene Signature

The inventors have shown the prognostic and predictive values of the malignancy-risk gene signature using three publically available NSCLC microarray datasets. Validation of the malignancy-risk gene signature in an independent dataset, larger or at least comparable with the Director's Challenging Consortium dataset is performed. Successful validation advances the malignancy-risk gene signature to the next level for the analytical and clinical validity. The malignancy-risk gene signature may be evaluated and a large-scale validation using microarray data from Total Cancer Care collected at the Moffitt Cancer Center may be completed. (Yeatman T J, Mule J, Dalton W S, et al. On the eve of personalized medicine in oncology. Cancer Res 2008; 68(18):7250-2; Koomen J M, Haura E B, Bepler G, et al. Proteomic contributions to personalized cancer care. Mol Cell Proteomics 2008; 7(10):1780-94).

Validating the MR signature uses the large clinico-genomic data in Total Cancer Care (TCC) cohort at Moffitt Cancer Center. There are 1,117 NSCLC patients with high quality gene microarray that received first course surgery at Moffitt and consented to TCC from 2006 to 2010 (˜46% male; 90% patients with age range of 50-80; more than 90% patients with smoking history). Among them, there are 855 early stage NSCLC patients (IA: 335; IB: 191; II; 156; IIIA: 173). Clinical information for array data is retrieved from the Moffitt TCC database including age, race, disease stage, grade, histopathologic sub-type, disease-fee, and overall survival as well as details of those clinical parameters used to evaluate response to therapy.

Computing the MR Score

To compute the MR score the gene symbol is used to find MR genes within TCC data (different microarray platforms between the MCLA and TCC cohorts are used). An averaged expression of probe sets are used to represent the gene level expression, if a gene has multiple probe sets. Prior to analysis, data is standardized by cornering at the mean and scaled by the standard deviation for each gone in both datasets. The MR score is constructed based cat the loading coefficients from the first principal component in the MCLA data. The same loading coefficients are also used to compute the MR scores for the TCC data. The median of the MR score in the MCLA data is used as the cutoff to form low and high MR score groups in the TCC data. This median-split MR score is then used for the following steps.

Validating Association With Overall Survival and Other Clinical Predictors

In order to determine if the MR signature can predict overall survival in TCC data, a log-rank test is used to compare the overall survival curves between the low and high MR score groups. Clinical predictors, such as age, gender, race, histopathologic sub-type, and disease-free survival are examined to determine correlation with the MR signature. Statistical methods such as one-way analysis of variance is used to test any differences of the continuous MR score among the groups for category variables (e.g., race) with the Tukey method to adjust for p value for pair-wise comparison. (Miller, R G, Simultaneous Statistical Inference 1981: Springer) Spearman correlation analysis is used to left any increasing/decreasing trend of the continuous MR score with stage, grade, smoking history, and other ordinal, variables, and the log-rank test is used to examine any difference of KM survival curves between low and high MR score groups for survival data (e.g., disease-free survival).

Testing the Predictive Effect

Since TCC is an observation cohort, it is likely that the patients receiving ACT had high-risk clinical characteristics such that ACT was recommended and they had worse survival than the ones without ACT. However, if, in high MR score patients, the ACT group could demonstrate better overall survival or comparable to the non-ACT group (outperform the high-risk clinical characteristics), the MR signature has a predictive effect. Patients are stratified into low and high MR score groups and the test is performed. The log-rank test is used to test for a survival difference between the ACT and Non-ACT groups. The Cox model is used to test for an interaction effect between ACT and the signature. The guideline for the evaluation of a predictive effect by Clark is applied. (Clark, G M, Prognostic factors versus predictive factors: Examples from a clinical trial of erlotinib. Molecular oncology, 2008, 1(4): p. 406-12)

The MR Signature's Effect Beyond the Conventional Predictors and the Interaction Effect

Subgroup analysis (e.g., stratify patients by TNM stage and analyze each subgroup to see if the MR signature predicts OS) is used to show that the MR signature is predictive beyond the conventional predictors. Conventional predictors are adjusted and interactions between the gene signature and ACT (and/or other predictors) are determined. The log-rank test is used to test if the MR signature predicts overall survival within different risk subgroups by clinical predictors (e.g., TNM stage). Multivariate Cox model is used to evaluate interaction effects between the malignancy-risk score and a clinical predictor after adjusting other clinical predictors.

Resilience and Transferability of the MR Signature

The MR score is created by the 1st principal component, which estimates weights (loading coefficients) for each MR gene to generate a weighted average score to represent the overall expression level for the signature. The MR score and the weights of the MR genes are compared from the MCLA to the TCC cohorts by Pearson correlation analysis. The number of MR genes showing significant association with OS and/or other clinical outcomes, as well as how many of the significant MR genes with the same trend effect are present between the MCLA and TCC cohorts is examined. This step fine tunes the signature by identifying the strongest MR genes for analytic validity, such as transforming the signature from fresh frozen to FFPE using TCC specimens. Statistical methods include the Cox proportional hazards model for univariate analysis and the q-value method for the false discovery rate (FDR). (Storey, J D, The positive false discovery rate: A Bayesian interpretation and the q-value. Annals of Statistics, 2003, 31: p. 2013-2035; Storey, J D and R Tibshirani, Statistical significance for genomewide studies. Proceedings of the National Academy of Sciences of the United States of America, 2003, 100(16): p. 9440-5)

Power Analysis

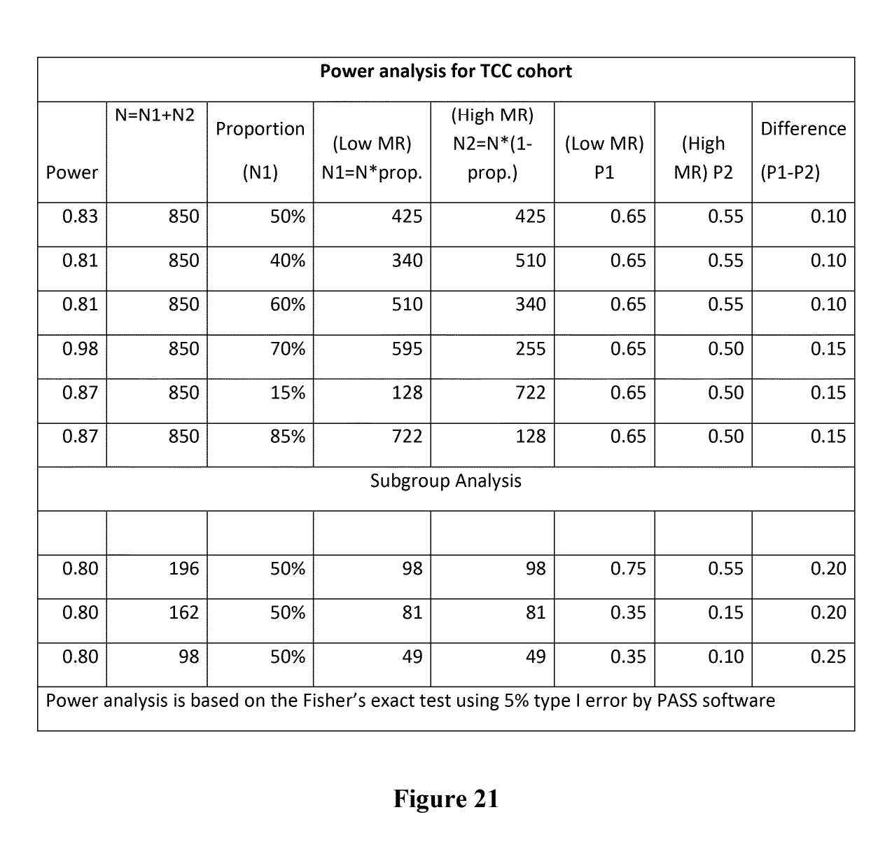

Preliminary data yielded a 5-year survival rate of 65% in the low MR group and 45% in the high MR group using the whole data in the MCLA cohort. Subgroup analysis also showed a 5-year survival rate of 75% (low MR) versus 56% (high MR) for stage IB, and 35% versus 8% for stage III. There are ˜850 NSCLC patients with stage IA-IIIA and gene expression data at TCC cohort. This sample size gives 83% power to detect a 5-year survival rate of 65% (low MR) versus 55% (high MR), a 10% difference, assuming equal sample size per group (n=425) and a two-sided 5% type I error based on the Fisher's exact test. The power remains above 80% when the sample size in the low MR group is 40% or 60% of the total sample size (unequal sample size between groups). Power for subgroup analysis is greater than 80% for a sample of 100 patients per group to detect a 20% difference of 5-year survival rate (75% versus 55% or 35% versus 15%). The sample size could be reduced to 50 subjects per group for an 80% power if a 25% difference of 5-year survival rate (35% versus 10%) is favorable. Detailed power analysis is given in FIG. 21.

Transferability of the MR Signature From Fresh Frozen (FF) Tissue Results to Formalin-Fixed and Paraffin-Embedded (FFPE) Tissues

Second, the microarray datasets described herein used fresh frozen (FF) tissues to extract RNA to measure gene expression. Although fresh frozen tissues are commonly used in research communities for microarray experiments, formalin-fixed and paraffin-embedded (FFPE) tissues are often collected in community-based hospitals with RT-PCR as a common technology to evaluate gene expression. The inventors validate the malignancy-risk gene signature in FFPE tissues, thus broadening the application of the signature in personalizing treatment care. A recent study has demonstrated feasibility of FFPE for gene signature development in NSCLC. (Xie Y, Ziao G, Coombes K, et al. Robust Gene Expression Signature from Formalin-Fixed Paraffin-Embedded Samples Predicts Prognosis of Non-Small-Cell Lung Cancer Patients. Clinical cancer research: an official journal of the American Association for Cancer Research 2011).

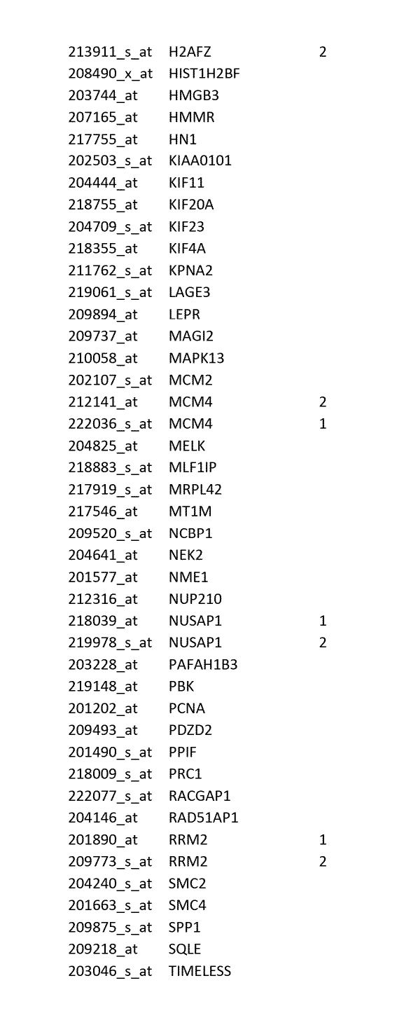

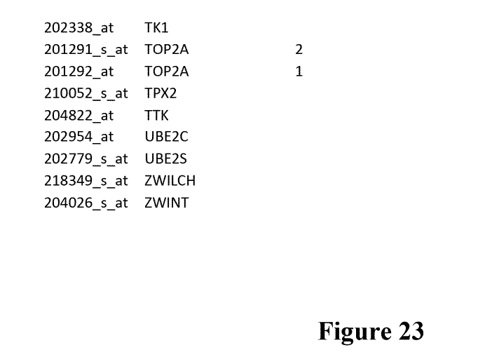

There are five steps involved in the translation-feasibility process: (1) The inventors select a subset of the 120 malignancy risk genes for translation—The inventors have identified 94-gene subset (102 probe sets in Affymetrix 133A chip) of the MR signature and successfully demonstrated its association with various clinical parameters (e.g., OS, grade, stage, and smoking status) in early-stage NSCLC patients. The inventors reduced to a 87-gene subset and showed its prognostic and predictive effects in three NSCLC microarray datasets. These 87 MR genes (FIG. 23) are converted into RT-PCR primer sets in step (2). In addition, two smaller subsets of the MR genes are investigated: a 60-gene subset and a 8-gene subset. We recently found that both subsets also yielded similar prognostic and predictive effects in early-stage NSCLC patients. The 60-gene subset was identified based on 5% FDR and the absolute value of loading coefficient>0.06 in the MCLA cohort. The 8-gene subset is comprised of the top 8 genes of the 60 MR genes with the highest absolute value of the loading coefficients (0.136-0.139).

(2) Translate the final selection of Affymetrix GeneChip probe sets into RT-PCR primer sets useful for FFPE material—The inventors have already demonstrated the feasibility of this step for 30 selected probes in the previous breast cancer study. (Chen, D T, A Nasir, A Culhane, C Venkataramu, W Fulp, R Rubio, T Wang, D Agrawal, S M McCarthy, M Gruidl, G Bloom, T Anderson, J White, J Quackenbush, and T Yeatman, Proliferative genes dominate malignancy-risk gene signature in histologically-normal breast tissue. Breast Cancer Res Treat 119(2): p. 335-46). The RT-PCR probes are designed to detect targets <80 bp in length. The inventors have found that while RNA degrades substantially when tissues are preserved in FFPE, RNA does not completely disappear but rather is reduced in size to smaller fragments that can be interrogated with proper RT-PCR probes. RT-PCR primers are designed that target small ˜80 bp fragments of RNA for the 87 genes. RNA extraction and RT-PCR experiments by the Tissue Core and Microarray Core are described as follows: RNA is extracted from FFPE material using the RecoverAll™ Total Nucleic Acid Isolation kit-optimized for FFPE samples (Ambion Inc., Austin, Tex.). 1-5 μm paraffin section for H&E staining and 4-20-μm paraffin unstained sections are cut. H&E slides are reviewed by a trained pathologist and the area with desired tissue type is marked with marker. 4 unstained sections mounted on glass slides are then macro-dissected with a scalpel using a marked H&E slide as a template. Harvested tissues are placed in RNase-free 2.0 ml Eppendorf tubes. Sections are then deparaffinized in xylene, at 50° C. for 3 minutes, centrifuged, and supernatant removed. Samples are washed several times in 100% ethanol. After the final wash, the samples are air-dried, and resuspended in digestion buffer with protease, followed by 3 hour incubation at 50° C. and 15 minute incubation at 80° C. RNA is purified by adding isolation additive and 100% ethanol, vortexed, and then passed through a filter cartridge. All RNAs are quantified by spectrophotometer. Reverse transcription (RT) for FFPE tissue is performed using Applied Biosystems High-Capacity cDNA Archive Kit following manufacturer's protocol for reverse transcription. Extracted RNA is reverse transcribed into cDNA, then preamplification is performed using the TaqMan® Pre-Amp Master Mix (2×) (Applied Biosystems) following the manufacturer's protocol. 50 ng of cDNA is used for each reaction. The TaqMan® Low Density Arrays are 384-well micro fluidic cards that enable quantitative real-time PCR reactions. These micro fluidic cards contain sequence specific primers/probes (TaqMan® Gene Expression Assays) that are pre-loaded into each of the wells on the cards. Each expression assay has an amplicon size less than 90 bases in length. Quantitative real-time PCR is carried out on the Applied Biosystems 7900 HT Real-Time PCR system;

(3) Perform RT-PCR analysis of 100 FFPE samples—FFPE blocks are linked to the frozen samples used to create the malignancy risk score from microarray analyses in the TCC cohort. Quartile-cutoffs of the MR score are used to form four subgroups based on microarray data from FF in the TCC cohort. For each subgroup, 25 FFPE samples are selected for the RT-PCR test. Specifically, in the low MR score group (below 25th percentile), patients with more than 5 years of overall survival are selected. In the high score group (above 75th percentile), patients with less than 2 years of overall survival are the targets. Since high MR score are associated with poor survival, the two groups have distinct MR score by RT-PCR. For the two intermediate MR (two groups: 1st quartile to median and median to 3rd quartile), patients with 2-5 years of overall survival are the top candidates. Inclusion of the two intermediate MR groups helps investigate the full spectrum of the MR score to see any strongly positive linear relationship between FF and FFPE. A total sample size of 100 samples will detect a correlation of 0.8 with lower bound at 0.72 and a correlation coefficient of 0.9 with lower bound at 0.86, using a two-sided 95% confidence interval by PASS software. Moreover, incorporation of overall survival allows us to evaluate clinical validity in RT-PCR based FFPE in step (5).

(4) Identify reliable control genes for normalization—described below with regard to the section on refining the malignancy-risk score system bar clinical application.

(5) Normalize RT-FCR data and perform principal component analysis to generate the malignancy-risk score using the 1st principal component in a similar fashion to the process used for Asymetrix data—Each new sample is assigned a malignancy risk score to determine the relative risk of death. The RT-PCR-based malignancy-risk score is tested to determine correlation with the microarray-based score by Pearson correlation analysis. A high correlation coefficient (>0.7) indicates that gene expressions collected from RT-FCR-based FFPE well represent the information derived from microarray-based FF. The log-rank test is then used to determine if the RT-PCR-based malignancy-risk score predicts overall survival and demonstrates that patients with a high malignancy-risk score tend to have shorter OS compared with those who base a low malignancy-risk score.

Refining the Malignancy-Risk Score System for Clinical Application

Several available gene expression profiling platforms, in clinical use such as Oncotype DX (Paik, S, S Shak, G Tang, C Kim, J Baker, M Cronin, F L Baehner, M G Walker, D Watson, T Park, W Hiller, E R Fisher, D L Wickerham, J Bryant, and N Wolmark, A multigene assay to predict recurrence of tamoxifen-treated, node-negative breast cancer. The New England journal of medicine, 2004 351(27); p. 2817-26) and MammaPrint (van 't Veer, L J, H Dai, M J van de Vijver, Y D He, A A Hart, M Mao, H L Peterse, K van der Kooy, M J Marton, A T Witteveen, G J Schreiber, R M Kerkhoven, C Roberts, P S Linsley, R Bernards, and S H Friend, Gene expression profiling predicts clinical outcome of breast cancer. Nature, 2002, 415(6871); p. 530-6) for breast cancer patients and ColoPrint (Salazar, R, R A Bender, S Bruin, G Capella, V Moreno Aguado, F Roepman, L van 't Veer, and R A Tollenaar, Development and validation of a robust high-throughput gene expression test (ColoPrint) for risk stratification of colon cancer Patients. Gastrointestinal Cancers Symposium, 2010. Orlando, Fla. Jan. 22-24, 2010 (abstr 295)) for colon cancer patients.

Determination of the Association of PCI With Clinical Parameters

Principal component analysis (PCA) is an unsupervised approach; thus the PCI may not correlate with clinical outcomes. Fortunately, in the preliminary data, the inventors demonstrated that the MR score (i.e., PCI) was associated with OS, grade, stage, and other clinical parameters in NSCLC. The clinical significant associations are tested to determine if they remain robust using various NSCLC microarray datasets. This is examined by re-sampling a portion of data (e.g., 90% data) over 1,000 times to show the clinical association. The inventors start with 90% data; if robust, the inventors decrease data size by 5%, and so on, until the clinical association becomes weak. This approach helps evaluate the robustness of the MR's clinical association and investigates its relationship with sample size.

Representation of PCI and Reliability of the Representation

The percentage of total variation of PCI in the MR signature ranges 40% to 50% in several NSCLC datasets from preliminary data. This is quite significant in contrast to other gene signatures, which is about 10-20% total variation for their PCI (personal observations). The re-sampling approach is used to test if PCI in the MR signature retains at least 40% total variation. PCI is compared to approaches based on multiple principal components (PCs), such as the top 5 PCs, or PCs accounted for 90% total variation (both approaches are common used in microarray analysis when PCA is engaged). The inventors determine if the percentage of total variation remains robust for the approach using multiple PCs and if the number of PCs remains robust for the approach based 90% total variation at various re-sampling schemes.

Reliability of the Loading Coefficients

Since the MR score is derived from the 1st principal component using loading coefficient (weight) for each MR gene to generate a weighted average score to represent the overall expression level for the signature, the inventors determine if the loading coefficients remain robust by the re-sampling approach described above in the determination of the association of PCI with clinical parameters. Specifically, for a given re-sampling scheme (e.g., 90% data), each re-sampling data yields a set of loading coefficients for PCI. This set of loading coefficients is compared to the ones using the whole data (100% data) by Pearson correlation analysis. Over 1,000 times of re-sampling yields a collection of 1,000 correlation coefficients. Since a correlation coefficient close to 1 indicates robustness of the loading coefficients, the inventors test to determine if the 25th percentile of the correlation coefficient reaches at least 0.9. In addition, the loading coefficients between various NSCLS datasets at each re-sampling step are compared to see if correlation remains high. The benchmark is 0.7 for the 25th percentile of the correlation coefficient.

Determination of the Loading Coefficient in the PCI as Being Indicative of Degree of Importance (e.g., Association With p Value)

If a gene with a large loading coefficient value has more statistical significance than one with a value close to 0, then by selecting important genes only and/or eliminating less relevant genes based on the value of loading coefficient assists in fine tuning the MR signature. Refinement of the MR signature to a smaller set of MR genes benefits the development of multi-gene assay development for clinical use since current commercial assays have less than 100 genes in their applications. The preliminary data has shown a strong relationship (r=−0.8) between the loading coefficient and p value with significant small p value in genes with large values (absolute value) of loading coefficients.

Identification of Reliable Control Genes for Calibration

A set of robust control genes is a must to normalize MR gene expression for multigene assay development. This strategy has been used in commercial assays, such as Oncotype DX using 5 control genes. Since there are many house-keeping genes embedded in microarray (e.g., beta-actin and GAPDH), the inventors utilize the information to explore various potential control genes for calibration using NSCLC microarray data. The inventors start with an individual control gene for normalization to see if the normalized MR score remains predictive of clinical outcomes and if the loading coefficients are robust. Then a set of top control genes for calibration are selected to see if performance could be enhanced. This step requires much trial and error to reach a solution (e.g., there are 45 combinations to select two genes from 10 control genes, 120 combinations to choose three genes, and more to choose four or five genes). The final set of control genes is validated by RT-PCR.

The malignancy risk signature is a “strong” signature that is reproducible using FFPE specimens. The inventors anticipate that a strong signature can be honed to ˜87 or fewer genes from the full MR genes (94 genes). The previous breast cancer study suggests that gene expression measured by the Asymetrix GeneChip correlates with that measured by RT-PCR technologies using the same samples. Thus, the RT-PCR method reproduces the signature and result in a cost effective, stand alone means to measure the malignancy risk score and may be translated to the clinic. Alternatively, microarray for FFPE specimens may be used to measure the MR gene expression. A recent study has demonstrated feasibility of FFPP using microarray for gene signature development in NSCLC. (Xie, Y, G Xiao, K Coombes, C Behrens, L M Solis, M G Raso, L Girard, H S Erickson, J A Roth, J V Heymach, C Moran, K D Danenberg, J D Minna, and Wistuba, I I, Robust Gene Expression Signature from Formalin-Fixed Paraffin-Embedded Samples Predicts Prognosis of Non-Small-Cell Lung Cancer Patients. Clinical cancer research: an official journal of the American Association for Cancer Research, 2011). A robust MR score system using the 1st principal component is used. Alternatively, additional principal components may be included using the supervised principal component method. (Bair, E and R Tibshirani, Semi-supervised methods to predict patient survival from gene expression data. PLoS biology, 2004, 2(4); p. E108) Other methods may also be used to predict overall survival, including random forests (Ishwaran, H, U B Kogalur, E H Blackstone, and M S Lauer, Random survival forests. Ann App. Statist, 2008. 2: p. 8441-860), and partial least squares (Nguyen, D V and D M Rocke, Partial least squares proportional hazard regression for application to DNA microarray survival data. Bioinformatics, 202, 18(12); p. 1625-32; Boolesteix, A L and K Strimmer, Partial least squares: a versatile tool for the analysis of high-dimensional genomic data. Briefings in bioinformatics, 2007, 8(1); p. 32-44).

Drug-Sensitivities Associated With MR Signature

The inventors have shown the MR signature as a prognostic and predictive signature in NSCLC patients. Clinical applications of the MR signature include whether its presence in a resected early-stage tumor indicates a benefit or a detriment effect for adjuvant chemotherapy following surgery. Since most cytotoxic chemotherapeutic drugs target proliferation, such as cisplatin to cause DNA damage and vinorelbine to inhibit mitosis, the malignancy-risk signature associates with some of the drugs. (Shapiro, G I, J G Supko, A Patterson, C Lynch, J Lucca, P F Zacarola, A Muzikansky, J J Wright, T J Lynch, Jr., and B J Rollins, A phase II trial of the cyclin-dependent kinase inhibitor flavopiridol in patients with previously untreated stage IV non-small cell lung cancer, Clin Cancer Res, 2001, 7(6); p. 1590-9; George, S, B S Kasimmis, J Cogswell, P Schwarzenberger, G I Shapiro, P Fidias, and R M Bukowski, Phase I study of flavopiridol in combination with Paclitaxel and Carboplatin in patients with non-small-cell lung cancer. Clin Lung Cancer, 2008, 9(3): p. 160-5). The inventors test if the MR signature predicts response to specific chemotherapeutic regiments such that the optional adjuvant chemotherapy can be used for a given patient and/or predict patients who will not respond to the drugs so that the treatment would not be recommended. In addition, cancer drugs targeting on molecular signaling pathways (e.g., gefitinib and erlotinib for inhibiting EGFR) are investigated to determine if the MR signature can predict the drug response. Since the NCI-60 cell line panel has been characterized with regard to thousands of potential therapeutic compounds, it provides so ideal database to discover what the inventors call “MR-associated drugs”. In addition, the broad and unrestricted microarray data have made in-silico validation of hypotheses accessible and feasible. The inventors explore various published microarray datasets which were measured before and after drugs in lung cancer cell lines to see any treatment effect related to the MR signature. The significant “MR-associated drugs” are validated in TCC samples by RTPCR.

Identification of Potential Drug Compound Sensitivities Associated With the MR Signature using NCI-60 Cell Line Data

NCI-60 cell line data is a very valuable rich dataset, but has very complicated structure, requiring significant effort for data acquisition and preprocessing, as well as identification of MR-associated drugs.

Data Acquisition and Preprocessing

The NCI-60 (Shoemaker, R H, The NCI60 human tumour cell line anticancer drug screen. Nature reviews. Cancer, 2006, 6(10); p. 813-23) consists of 59 human cancer cell lines derived from 9 tissue types, including 9 NSCLC cell lines. Gene expression is used and correlated to drug response to identify chemical compounds to which the MR-signature predicts sensitivity.

(a) Drug Sensitivity Data:

There are ˜43,000 compounds with drug response data available (G150, TGI, and LC50; December 2010 updated) for the NCI-60 panel by the NCI's Developmental Therapeutics Programs. G150 is used as the primary measurement because of its common use and the lowest concentrations of substances for the observed effect. Various normalization and quality control approaches are used to preprocess the drug sensitivity data for the NCI-60 panel to avoid “garbage in and garbage out”. At least four approaches are considered to preprocess data with additional methods included as time progresses. Normalization is performed across all the cell lines for each compound using two methods: (1) rank method by ranking the G150 value (Ring, B Z, S Chang, L W Ring, R S Seitz, and D T Ross, Gene expression patterns within cell lines are predictive of chemosensitivity. BMC genomics, 2008, 9: p, 74); (2) standardization of log (G150) by centering at mean and scaled by standard deviation (Staunton, J E, D K Slonim, H A Coller, P Tamayo, M J Angelo J Park, U Scherf, J K Lee, W O Reinhold, J N Weinstein, J P Mesirov, E S Lander, and T R Golub, Chemosensitivity prediction by transcriptional profiling. Proceedings of the National Academy of Sciences of the United States of America, 2001, 98(19): p. 10787-92). The inventors use non-normalized data, log(GI50), for analysis. (Lee, A C, K Shedden, G R Rosania, and G M Crippen, Data mining the NCI60 to predict generalized cytotoxicity. Journal of chemical information and modeling, 2008, 48(7); p. 1379-88; Ma, Y, Z Ding, Y Qian, Y W Wan, K Tosun, X Shi, V Castranova, E J Harner, and N L Guo, An integrative genomic and proteomic approach to chemosensitivity prediction. International journal of oncology, 2009, 34(1): p. 107-15). In parallel, the drug sensitivity data is dichotomized into “sensitive” and “resistant” based on a cutoff of standard deviation (sensitive: <mean-cutoff, resistant: >mean+cutoff, and intermediate: is within the cutoff; Ma et al. used 0.5 SD as the cutoff and Staunton et al. used 0.8 SD as the cutoff). In addition, several metrics are considered to filter out poor quality data; GI50 available in more than 75% of cell lines, standard deviation greater than 0.1, and/or the means of log(GI150) less than −4 (indicating some quantiative level of drug response activity).

(b) Gene Microarray Data and the MR Signature

Gene expression data: The inventory have identified six gene expression datasets: GSE5846 (Affymetrix 133 A chip) and GSE22821 (Agilent Whole Human Genome Oligo Microarray) (Liu, H, P D'Andrade, S Fulmer-Smentek, P Lorenzi, K W Kohn J N Weinstein, Y Pommier, and W C Reinhold, mRNA and microRNA expression profiles of the NCI-60 integrated with drug activities. Molecular cancer therapeutics, 2010, 9(5); p. 1080-91), GSE32474 (Affymetrix U133 Plus 2.0) GSE7505 (NHGRI Homo sapiens 6K), GSE7947 (spotted DNA/cDNA array from Stanford Functional Genomics Facility), and GSE28709 (Rosetta/Merck Human RSTA Affymetrix 1.0 microarray). All datasets are able to link to the NCI 60 drug sensitivity data. In addition, the GSE7505 dataset provides another opportunity to evaluate radiation sensitivity. The GSE28709 dataset has gene expression data for 93 long cancer cell lines with 5 cell lines overlapped with the NCI60 cell line: A549, H226, H23, H460, and H522. These 5 cell lines are analyzed for this dataset. Appropriate normalization methods are used to adjust for background noise (e.g., RMA[34] for Affymetrix gene chip and GeneSpring software for Agilent microarray).

Malignancy-risk gene signature: Due to different microarray platforms, malignancy-risk gene signature could be slightly changed. Two approaches are used to address this issue. For Affymetrix data, because of the same platform, the 102 probe sets of the malignancy-risk genes are used for analysis. For non-Affymetrix data, gene symbol is used to find malignancy-risk genes (The GSE7947 dataset provides only the gene bank accession number extra effort will be implemented to link to gene symbols. Two types of data are analyzed: probe set level and gene level. For probe set level data, the 102 probe sets of the malignancy-risk signature are used for analysts in Affymetrix data only. For gene level data, an averaged intensity of probe sets is used to represent the gene expression if a gene has multiple probe sets. Any probe set with missing value was excluded. The gene level data is analyzed in both Affymetrix and non-Affymetrix platforms. An overall malignancy-risk score is generated by principal component analysis to reflect the combined expression of the malignancy-risk genes. Specifically, the first principal component (a weighted average expression by loading coefficient among the malignancy-risk genes) is used, as it accounts for the largest variability in the data, to represent the overall expression level for the signature. This approach has been successfully applied to the breast and lung cancer studies for the malignancy-risk gene signature. The MR score is then used to identify MR-associated compounds.

Identification of MR-Associated Drugs

Chemotherapeutic drugs most commonly used in ACT of NSCLC are investigated, specifically platinum agents, taxanes and gemacitabine. Since these drugs interfere with cell division by mitosis and the MR is a proliferative-enriched gene signature, the MR signature is a biological indicator reflecting and predicting drug sensitivity. Drugs targeting on molecular signaling pathways (e.g., gefitinib and erlotinib) are also investigated to determine the MR signature correlation to the drug response. A recent report of a randomized phase III trial showed that erlotinib or gefitinib are superior to platinum-based chemotherapy for EGFR-mutant NSCLC. (Zhou, C, Y L Wu, G Chen, J Feng, X Q Liu, C Wang, S Zhang, J Wang, S Zhou, S Ren, S Lu, L Zhang, C Hu, Y Luo, L Chen, M Ye, J Huang, X Zhi, Y Zhang, Q Xiu, J Ma, and C You, Erlotinib versus chemotherapy as first-line treatment for patients with advanced EGFR mutation-positive non-small-cell lung cancer (OPTIMAL, CTONG-0802): a multicentre, open-label, randomised, phase 3 study. The lancet oncology, 2011, 12(8); p. 735-42). In addition, many other compounds are explored to find any MR-associated compounds with potential to personalize the treatment. The 9 NSCLC cell lines from the NCI-60 panel are examined to test if any drug associates with the MR signature specifically to the NSCLC cell lines. Then the analysis extends to the entire NCI-60 panel.

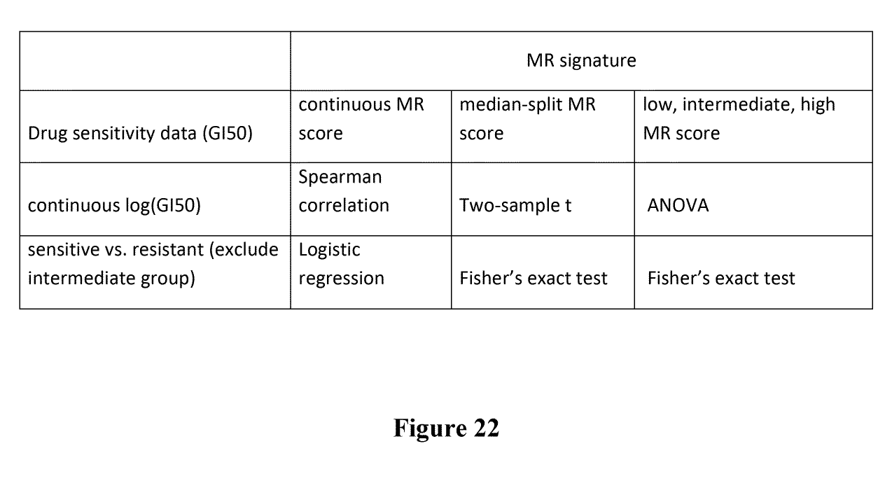

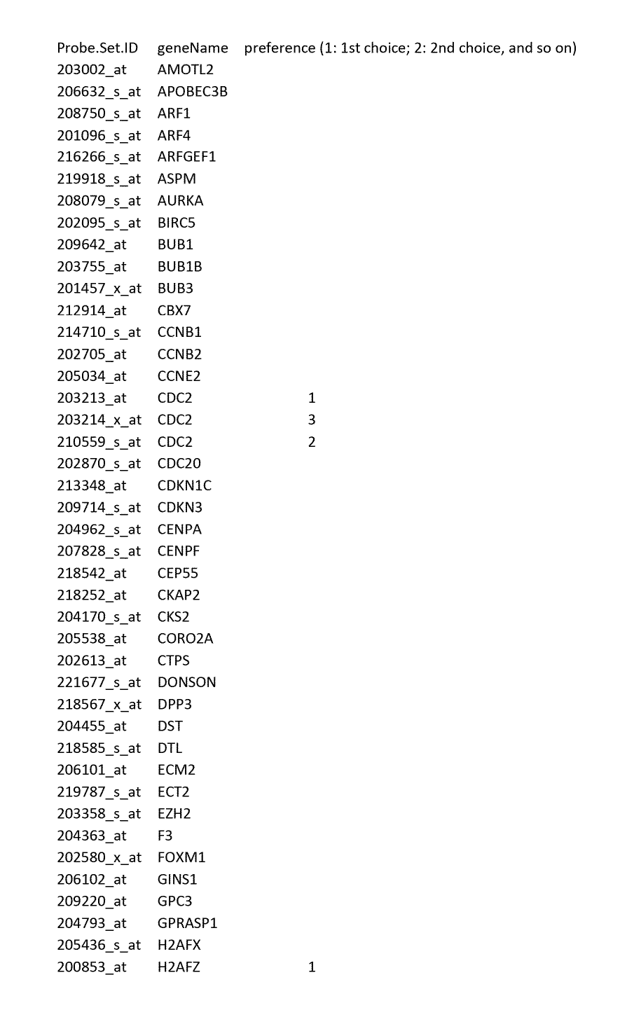

To detect which drugs may affect the MR signature, various statistical methods are employed to analyze different types of drug sensitivity data in NCI-60 panel and MR scores and are summarized in FIG. 22. The q-value is used to estimate false discovery rate (FDR) for each test statistic (a q-value of 0.05 indicates five expected false positives for every 100 significant tests). This is important especially when assessing tens of thousands of compounds and assessing if it could be a valuable early discovery tool. Various q value cutoffs are explored, but not greater than 20% FDR, to see how many of significant compounds associated with the MR signature.

Validation of significant MR-associated compounds using publically available datasets (in-silico validation) and in human tissues by RT-PCR.

Once significant MR-associated compounds are identified, the MR signature has biological effect to these compounds is tested. The MR-associated compounds are evaluated by in-silico validation from publically available datasets. Significant effort is made to select appropriate datasets with experiments such as comparing compound-treated cell lines versus control (untreated) or new IC50 of specific drugs in lung cancer cell lines. The significant MR-associated compounds verified by in-silico validation are confirmed by RTPCR in FFPE tissues.

In-Silico Validation

Thousands and thousands of gene expression array data have been deposited at various public repositories, such as NCBI's Gene Expression Omnibus and EBI's ArrayExpress with more than 600,000 array data for each site (updated in October 2011; 100,000 more since January 2011). The broad and unrestricted microarray data have made in-silico validation of hypotheses accessible and feasible. For this invention, more than 1,000 microarray datasets have potential for performing in-silico validation (e.g., data related to the keyword “non-small cell lung cancer cell line” at GEO). First, appropriate datasets are identified for validation by reading the ankle (at least abstract and experimental design). At this moment, more than 10 potential useful datasets have been identified. (1) GSE4342 of gefitinib sensitivity in NSCLC cell lines (Coldren, C D, B A Helfrich, S E Witta, M Sugita, R Lapadat, C Zeng, A Baron, W A Franklin. F R Hirsch, M W Getaci, and P A Bunn, Jr. Baseline gene expression predicts sensitivity to gefitinib in non-small cell lung cancer cell lines. Molecular cancer research: MCR, 2006, 4(B); p. 521-8.); (2) GSE10089 of anti-tumor activity of histone deacetylase inhibitors in non-small cell lung cancer cells (Miyanaga, A, A Gemma. R Noro, K Kataoka, R Matsuda, M Nara, T Okano, M Seike, A Yoshimura, A Kawakami, H Uesaka, H Nakae, and S Kudoh, Antitumor activity of histone deacetylase inhibitors in non-small cell lung cancer cells: development of a molecular predictive model. Molecular cancer therapeutics, 2008, 7(7); p. 1923-30); (3) GSE8332 of death receptor o-glycosylation controls tumor-cell sensitivity to the proapoptotic ligand Apo2L/TRAIL (Wagner, K W, E A Punnoose, T Januario, D A Lawrence, R M Pitti, K Lancaster, D Lee, M von Goetz, S F Yee, K Totpal, L Huw, V Katta, G Cavet, S G Hymowitz, L Amler, and A Ashkenazi, Death-receptor O-glycosylation controls tumor-cell sensitivity to the proapoptotic ligand Apo2L/TRAIL. Nature medicine, 2007, 13(9); p. 1070-7); (4) GDS1204 of lung cancer cell line response to motexafin gadolinium; time course (Magda, D, P Lecane, R A Miller, C Lepp, D Miles, M Mesfin, J E Biaglow, V V Ho, D Chawannakul, S Nagpal, M W Karaman, and J G Hacia, Motexafin gadolinium disrupts zinc metabolism in human cancer cell lines. Cancer research, 2005, 65(9); p. 3837-45); and (5) GSE4127 of anticancer drug clustering in lung cancer based on gene expression profiles and sensitivity database (Gemma, A, C Li, Y Sugiyama, K Matsuda, Y Seike, S Kosaihira, Y Minegishi, R Noro, M Nara, M Seike, A Yoshimura, A Shionoya, A Kawakami, N Ogawa, H Uesaka, and S Kudoh, Anticancer drug clustering in lung cancer based on gene expression profiles and sensitivity database. BMC cancer, 2006, 6; p. 174); (6) GSF6410 of Cisplatin-induced gene expression changes in A549 NSCLC cells (Ameida, G M, T L Duarte, P B Farmer, W P Steward, and G D Jones, Multiple end-point analysis reveals cisplatin damage tolerance to be a chemoresistance mechanism in a NSCLC model; implications for predictive testing. International journal of cancer. Journal international du cancer, 2008, 122(8); p. 1810-9).

Second, array data and clinical information is extracted. Before downloading datasets, determination of what type of microarray platform was used and how data were pre-processed and normalized is needed. Re-construction of clinical/experimental data is needed for statistical analysis.

Third, the malignancy-risk gene signature is validated. Due to various microarray platforms, array data from the cell lines may not have the complete list of MR genes. The gene symbol is used to identify all possible features related to MR genes and take an average of multiple features which interrogate a same MR gene to represent the expression for the MR gene. The next step is to standardize each MR gene across samples by centering at mean and scaled by standard deviation. The coefficients (PCI loading coefficients derived, from the Director Challenging data or NCI-60 panel) are used to calculate MR score for the cell line data to determine the MR score predicting drug sensitivity by various statistical methods depending on experiment design (e.g., two-sample t-test for treated versus control).

### Validation of Significant MR-Associated Compounds in FFPE Tissues by RT-PCR

The top 10 common chemotherapy drugs at TCC cohort are Carboplatin, Paclitaxel, Gemcitabine, Docetaxel, Alimta, Tarceva, Cisplatin, Vinorelbine, Avastin, and Iressa. The top 2 chemotherapy drugs based on the results from above are selected for RT-PCR validation. For each drug, 25 responded and 25 non-responders are selected to compare the MR score between the two groups. This sample gives 93% power to detect one unit of effect size for the MR score given a 5% type I error and a two-sided two-sample t-test. The one unit effect size translates into one unit difference of the MR score between the two groups given the common standard deviation as 1. If the performance in drug response is similar to the one in grade (well versus moderate-differentiation) or stage (IA versus II) in FIG. 18, the sample is sufficient to detect the difference.

The preliminary results (FIG. 20) have shown at least two drugs as potential “MR-associated drugs”: Cisplatin and Vinorelbine. Since many patients it TCC cohort received multiple chemotherapy drugs, it will be difficult to select enough patients with simple drug. Due to the limitation, patients with simple drug are first selected and then patients with double drugs are selected, and so on until it reaches the desired sample size. Also, if response data is limited, disease-free survival is used as the outcome variable.

In summary: the malignancy-risk gene signature is useful to improve prediction of OS in NSCLC patients and is a tool to more accurately identify patients who will benefit front adjuvant therapy after surgical resection.

In the preceding specification, all documents, acts, or information disclosed do not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described.

What is claimed is:

1. A method of diagnosing cancer comprising:
obtaining a sample tissue;
obtaining a malignancy-risk score, wherein the malignancy-risk score is formed by
collecting at least one gene expression level;
weighting the expression level; and
applying the least one gene expression level and weighting the expression level to the following formula Σwᵢxᵢ, where xi represents gene i expression level wi is the corresponding weight (loading coefficient);
wherein the malignancy-risk score is indicative of clinical diagnosis of the sample tissue for cancer.

2. The method of claim 1, further comprising calculating gene expression values using the robust multi-array average algorithm.

3. The method of claim 1, further comprising using a probe set to detect the at least one gene expression level for lung cancer or breast cancer.

4. The method of claim 1, further comprising analysing at least one clinical variable in concert with the malignancy-risk score, wherein the at least one clinical variable is TNM stage, grade, histologic grade, or smoking history.

5. The method of claim 4, wherein the TNM stage variables analyzed are pathologic N stage or pathologic T stage.

6. The method of claim 4, wherein the analysis is conducted using multivariate Cox proportional hazards regression analysis.

7. The method of claim 1, wherein the at least one gene expression level is from at least one malignancy-risk gene.

8. The method of claim 1, wherein a low malignancy-risk score correlates with better survival.

9. A method of predicting the response of a subject to therapy for lung cancer comprising:
obtaining a sample tissue;
obtaining a malignancy-risk score, wherein the malignancy-risk score is formed by
collecting at least one gene expression level;
weighting the expression level; and
applying the least one gene expression level and weighting the expression level to the following formula Σwᵢxᵢ, where xi represents gene i expression levels wi is the corresponding weight (loading coefficient);
wherein the malignancy-risk score is indicative of clinical diagnosis of the sample tissue for cancer.

10. The method of claim 9, further comprising calculating gene expression values using the robust multi-array average algorithm.

11. The method of claim 9, further comprising using a probe set to detect the at least one gene expression level for lung cancer or breast cancer.

12. The method of claim 9, further comprising analysing at least one clinical variable in concert with the malignancy-risk score, wherein the at least one clinical variable is TNM stage, grade, histologic grade, or smoking history.

13. The method of claim 12, wherein the TNM stage variables analyzed are pathologic N stage or pathologic T stage.

14. The method of claim 12, wherein the analysis is conducted using multivariate Cox proportional hazards regression analysis.

15. The method of claim 9, wherein the at least one gene expression level is from at least one malignancy-risk gene.

16. The method of claim 9, wherein a low malignancy-risk score correlates with a patient that may benefit from adjuvant chemotherapy (ACT).
